(19) United States
(12) Patent Application Publication
(10) Pub. No.: US 2026/0345813 A1
(43) Pub. Date: Nov. 5, 2026

(54) Surface Display of Immunotherapeutics on Extracellular Vesicles and Uses Thereof

(71) Applicant: NATIONAL UNIVERSITY OF SINGAPORE, Singapore (SG)

(72) Inventors: Thi Nguyet Minh Le, Singapore (SG); Migara Kavishka Jayasinghe, Singapore (SG); Hoang Diem Phuong Nguyen, Singapore (SG)

(21) Appl. No.: 19/484,855

(22) PCT Filed: May 17, 2024
(86) PCT No.: PCT/SG2024/050331

§ 371 Date: Nov. 14, 2025

(30) Foreign Application Priority Data
May 17, 2023 (SG) 10202301385W

(51) Int. Cl. A61K 47/69; A61K 47/68; A61P 35/00

(52) U.S. Cl. CPC A61K 47/6901; A61K 47/6849; A61P 35/00

(57) ABSTRACT

Disclosed herein are extracellular vesicles and their uses in immunotherapy. Also disclosed herein are methods to generate the extracellular vesicles.

### CROSS-REFERENCE TO RELATED APPLICATIONS

This application claims the benefit of priority of Singapore provisional application no. 10202301385W, filed 17 May 2023, the contents of it being hereby incorporated by reference in its entirety for all purposes.

### FIELD OF THE INVENTION

The present invention relates generally to the field of molecular biology. In particular, the present invention relates to engineered extracellular vesicles and the use of the same.

### BACKGROUND OF THE INVENTION

Cancer is the second leading cause of non-communicable disease deaths worldwide, hence posing a great challenge in public health. Besides standard treatments, including chemotherapy, radiation, and surgery, immunotherapy has become a pillar for cancer treatments.

Immunotherapy has been one of the major breakthroughs in cancer therapy. In the last decade, the emergence of multiple forms of cancer immunotherapy has come to encompass the use of immune checkpoint inhibitors (ICIs), agonistic antibodies, and recombinant cytokines. Despite the efficacy of these therapeutics at activating the immune system and suppressing cancer progression, these immunotherapies often have adverse side effects or immune-related adverse events (irAEs). Moreover, combination immunotherapy has been shown to be able to cause significant off-target toxicities, including liver toxicity and the induction of autoimmune responses. One of the key reasons for such off-target toxicities is the non-specific nature of these immunotherapies that often circulate throughout the body, inducing unwanted immune activation outside of the tumour microenvironment.

Current delivery methods for immunotherapies are often hampered by unwanted immune activation outside of the tumour microenvironment. Various attempts have been made to develop new delivery strategies with the aim of reducing the toxicity of immunotherapies. However, these delivery strategies often result in decreased efficacy of immune activation in the tumour and may not be always applicable to all classes of anti-cancer immunotherapies.

There is therefore an unmet need for improving delivery methods for immunotherapies.

### SUMMARY OF THE INVENTION

In one aspect, the present disclosure refers to a nanoscale extracellular vesicle for immunotherapy, wherein one or more types of immunomodulatory molecules is/are anchored to the nanoscale extracellular vesicle, wherein the types of immunomodulatory molecules are selected from the group consisting of agonistic antibodies, antagonistic antibodies, cytokines, chemokines, small molecules, agonistic ligands, and combinations thereof, wherein the extracellular vesicle comprises click chemistry handles.

In another aspect, the present disclosure refers to a method of obtaining a nanoscale extracellular vesicle for immunotherapy as disclosed herein. The method comprising: a. isolating nanoscale extracellular vesicles from a cell or a compound capable of forming extracellular vesicles; b. attaching a click chemistry handle to the nanoscale extracellular vesicle surface; c. attaching a corresponding click chemistry handle to the immunomodulatory molecule; and d. conjugating the click chemistry handle to the corresponding click chemistry handle of step c; thereby anchoring the immunomodulatory molecules to the surface of the nanoscale extracellular vesicle.

In yet another aspect, the present disclosure refers to a nanoscale extracellular vesicle obtained using the method as disclosed herein.

In one aspect, the present disclosure refers to a method of treating or preventing a disease, the method comprising administering a composition comprising one or more nanoscale extracellular vesicles as disclosed herein to a subject.

### BRIEF DESCRIPTION OF THE DRAWINGS

The invention will be better understood with reference to the detailed description when considered in conjunction with the non-limiting examples and the accompanying drawings, in which:

FIG. 1 shows that iEDDA-mediated conjugation allows efficient extracellular vesicle (EV) surface functionalisation. FIG. 1(a) shows a schematic depicting iEDDA-mediated EV surface functionalisation. FIG. 1(b) shows a histogram of single EV flow cytometric analysis, assessing the conjugation efficiency of varying concentrations of fluorescent AZ488-TCO with MTet-EVs. FIG. 1(c) shows a dot plot of single EV flow cytometric analysis, assessing Biotin-TCO conjugation on MTet-Evs. EVs were co-stained with GPA antibody to accurately gate RBCEVs. FIG. 1(d) shows a graph demonstrating the percentage of RBCEVs conjugated with biotin, as assessed in (c). FIG. 1(e) shows the results of western blot assessing the conjugation efficiency of increasing concentrations of Biotin-TCO with MTet-EVs. FIG. 1(f) shows a graph of quantification of Biotin-TCO probes conjugated per EV determined using competition ELISA. FIG. 1(g) shows a graph demonstrating the percentage yield of the iEDDA reaction over time assessed by the reaction between MTet-EVs with Biotin-TCO. FIG. 1(h) shows the relative efficiency of iEDDA-mediated conjugation assessed using western blot comparing banding intensity of Biotin-STP EVs and Biotin-TCO-MTet-EVs. FIG. 1(i) shows the western blot results of the comparison of iEDDA-mediated conjugation with enzymatic ligation. FIG. 1(j) shows the western blot results of the comparison of iEDDA-mediated conjugation with SPAAC-mediated click chemistry. FIG. 1(d), FIG. 1(f), FIG. 1(g), and FIG. 1(h) represent data from 3 individual replicate prepared from separate batches of EVs. EV(s): extracellular vesicle(s), MTet: Methyltetrazine, TCO: trans-cyclooctene, iEDDA: inverse electron demand Diels-Alder, SPAAC: strain-promoted azide-alkyne cycloaddition. P-value (*** P<0.001) was determined using Student's one-tail t-test. In FIG. 1(e), FIG. 1(h), FIG. 1(i), and FIG. 1(j), molecular weights of protein markers in kDa are shown on the left.

FIG. 2 shows that iEDDA-functionalised EVs display proteins on the EV surface at high copy number. FIG. 2(a) shows a single EV flow cytometry histogram depicting the conjugation of a TCO-labelled rat IgG onto MTet-labelled EVs. FIG. 2(b) shows a graph demonstrating the percentage of EVs conjugated with rat IgG as determined in FIG. 2(a). FIG. 2(c) shows the western blot analysis assessing the conjugation of IgG onto EVs via iEDDA-mediated conjugation. The western blot was run under reducing conditions. FIG. 2(d) shows flow cytometry dot plots from single EV flow cytometric analysis of MTet-EVs conjugated with pairs of TCO-labelled proteins. FIG. 2(e) shows a graph demonstrating the copy number of IgG conjugated per EV, as determined using a total Rat IgG ELISA kit. FIG. 2(f) shows the size distribution profiles of unmodified of iEDDA conjugated EVs determined using NTA. The mean diameter is noted on each histogram. P-value (*** P<0.001) was determined using Student's one-tail t-test. In FIG. 2(c), molecular weights of protein markers in kDa are shown on the left. EV(s): extracellular vesicle(s), MTet: Methyltetrazine, TCO: trans-cyclooctene, iEDDA: inverse electron demand Diels-Alder, NTA: nanoparticle tracking analysis.

FIG. 3 shows that EV-mediated crosslinking of ligands enhances signalling compared to equivalent doses of free ligands. FIG. 3(a) shows a schematic depicting the differences in relative signalling efficiency of free agonistic CD137 antibodies as compared to a similar number of antibodies conjugated on a single EV. FIG. 3(b) shows a histogram overlay of EV-cell association assay comparing the relative binding affinity of CD137 antibody-conjugated EVs (EV-CD137 Ab) to EVs conjugated with an isotype control antibody (EV-Iso Ab). EV binding was detected using flow cytometry staining for GPA, an RBCEV specific surface marker. FIG. 3(c) shows a bar graph displaying the summary of the EV-cell association assay in FIG. 3(b). FIG. 3(d) shows a bar graph displaying the results of IFN-γ ELISA for supernatants of T-cells treated with EV-conjugated or free CD137 agonistic antibody. FIG. 3(e) shows a line graph demonstrating the relative effect of increasing doses of CD137 agonistic antibody-conjugated EVs displaying varying copy numbers of antibody per EV. The concentration in μM depicts the concentration of TCO-antibody incubated with MTet-EVs. FIG. 3(f) shows a bar graph of interferon gamma release from activated T-cells following stimulation with free (soluble) mouse CD137L or EV-conjugated CD137L. For FIG. 3(c), FIG. 3(d), FIG. 3(e), and FIG. 3(f), each replicate was performed using EVs from individual donors. Student's one-tailed t-test: ns—not significant, *P<0.05, *** P<0.001. EV(s): extracellular vesicle(s), GPA: Glycophorin A, RBCEV: red blood cell-derived extracellular vesicle, MTet: Methyltetrazine, TCO: trans-cyclooctene, CD137L: antibodies against cluster of differentiation 137, CD137L: CD137 Ligand.

FIG. 4 shows that EV-mediated crosslinking of agonistic αCD3 antibodies enhances signalling compared to free antibodies. FIG. 4(a) shows the results of EV-cell association assay comparing the relative binding affinity of Aco600-labelled αCD3 antibody-conjugated EVs (EV-CD3 Ab) to EVs conjugated with an isotype control antibody (EV-IgG) as determined by flow cytometric analysis of median fluorescent intensity (MFI) of Aco600 in CD3⁺ T cells in human PBMCs. FIG. 4(b) to FIG. 4(c) show bar graphs demonstrating the IFN-γ (left panel) and IL-2 (middle panel) ELISA for supernatants of human non-activated or activated PBMCs treated with EV-conjugated or free αCD3 agonistic antibody. Percentage of CD69⁺ immune cells (right panel) in human non-activated or activated PBMCs treated with EV-conjugated or free αCD3 agonistic antibody, as determined by flow cytometry. ANOVA with Tukey's multiple comparisons: ns—not significant, *P<0.05, ** P<0.01, *** P<0.001 and *** P<0.0001. EV(s): extracellular vesicle(s), Aco600: Acoerela fluorescent dye, αCD3 antibodies: anti-cluster of differentiation 3 antibodies, ELISA: enzyme-linked immunosorbent assay, PBMCs: peripheral blood mononuclear cells, IFN-γ: interferon gamma, CD69⁺: cluster of differentiation 69 positive.

FIG. 5 shows that the display of multiple complementary ligands in cis is superior to trans display. FIG. 5(a) shows a schematic illustrating the experimental design utilised to compare the relative signalling efficiency of cis or trans EVs as compared to free antibodies. FIG. 5(b) shows a line graph depicting the mean fluorescent intensity of CD69 on T-cells over a period of 5 days following treatment with each of the treatments. FIG. 5(c) shows a line graph depicting the mean fluorescent intensity of on T-cells over a period of 5 days following treatment with each of the treatments. A positive control of Beads-Cis is included for reference which consisted of Dynabeads conjugated with the same ligands in a similar orientation as shown in EV-Cis. FIG. 5(d) shows a line graph demonstrating the proliferation of T-cells following stimulation with each of the treatments tracked over a period of 6 days using alamarBlue assay. FIG. 5(e) shows histograms of CellTrace proliferation assay conducted on T-cells on day 3 following stimulation with each treatment. The unstimulated cells are overlaid with each histogram in black for comparison. FIG. 5(f) shows a bar graph demonstrating the IFN-γ ELISA of the supernatant of T-cells 6 days post treatment. FIG. 5(g) shows a bar graph demonstrating the percentage of Granzyme B positive CD8⁺ T-cells in each treatment condition on day 6 assesses using intracellular flow cytometry. Student's one-tailed t-test: ns—not significant, *P<0.05, *** P<0.001. For FIG. 5(b), FIG. 5(c), and FIG. 5(d), significance marks are shown to denote significant differences between EV-Cis and EV-Trans. ELISA: enzyme-linked immunosorbent assay, IFN-γ: interferon gamma, CD69: cluster of differentiation 69.

FIG. 6 shows that bispecific EV-Cis (BEVs) conjugated with αCD3 and αPD-L1 antibodies target T cells and tumour cells with high efficiency and specificity. EV-cell association assay comparing the relative binding affinity of Aco-600-labelled αCD3 antibody-conjugated EVs (EV-CD3 Ab) to EVs conjugated with an isotype control antibody (EV-IgG) as determined by flow cytometric analysis of median fluorescent intensity of Aco-600 in CD3⁺ T cells in human PBMCs (FIG. 6(a)), PD-L1⁺ H1975 cells (FIG. 6(b)), and PD-L1⁺ H441 cells (FIG. 6(c)). ANOVA with Tukey's multiple comparisons: ns—not significant, *P<0.05, ** P<0.01, and *** P<0.001. Aco600: Acoerela fluorescent dye, αCD3 antibodies: anti-cluster of differentiation 3 antibodies, αPD-L1: anti-programmed death-ligand 1.

FIG. 7 shows that multifunctional immunomodulatory extracellular vesicles (EVs) display superior ex vivo tumour cell suppression compared to free ligands. FIG. 7(a) shows the design of the ex vivo tumour cell killing assay. An ImmEV (also referred to as EV-Ligand) was developed displaying a complementary combination of immunomodulatory ligands in cis. Each component on the EV was designed to target specific interactions between T-cells and other cells in the tumour microenvironment. Agonistic CD3, CD137 antibodies and IL-2 enhance T-cell activation, survival and proliferation while antagonistic PD-1 and CTLA-4 antibodies inhibit common immune checkpoint blockades, preventing interactions between T-cell PD-1 and tumour cell PD-L1 and T-cell CTLA-4 with DC-displayed B7-1/B7-2 facilitating efficient T-cell mediated anti-tumour immunity. Readouts of T-cell activation, cytotoxicity and tumour cell viability was determined as shown on the panel in the bottom right. FIG. 7(b) shows the outline of the ex vivo tumour cell killing assay. A B16-F10 cell mouse melanoma cell line expressing a firefly luciferase reporter was used to monitor tumour cell death. FIG. 7(c) to FIG. 7(d) show bar graphs demonstrating the percentage of tumour cell death following co-culture with either splenocyte (FIG. 7(c)) or sorted CD8 T-cells (FIG. 7(d)) as the stated ratios. FIG. 7(e) shows a bar graph demonstrating the expression of granzyme B in CD8 T-cells following co-culture and treatment. FIG. 7(f) shows a bar graph demonstrating the Ki-67 expression in total T-cells at the endpoint of the experiment. ImmEV: immunomodulatory extracellular vesicle, CD3: Cluster of Differentiation 3, CD137: Cluster of Differentiation 137, IL-2: interleukin-2, PD-1: programmed death-1, CTLA-4: Cytotoxic T-Lymphocyte-Associated Protein 4, DC: dendritic cell, CD8: Cluster of Differentiation 8.

FIG. 8 shows that bispecific extracellular vesicles (BEVs) activate T cells and elicit potent cytotoxicity against human lung cancer cells. FIG. 8(a) to FIG. 8(b) show bar graphs of the quantification of IFN-γ (left panel) and IL-2 (middle panel) using ELISA in the supernatants of human non-activated (FIG. 8(a)) or activated PBMCs (FIG. 8(b)) treated with free EVs, EVs conjugated with αCD3 and αPD-L1 antibodies (BEV) or with IgG (EV-IgG) or the free αCD3 and αPD-L1 antibodies (free ligand) for 48 hours (n=3). FIG. 8(c) shows a bar graph demonstrating the percentage of CD69⁺ and CD25⁺ on CD4⁺ and CD8⁺ T cells in non-activated human PBMCs treated with free EVs, BEVs, EV-IgG or free ligands, as determined by flow cytometry (n=3). FIG. 8(d) shows a bar graph demonstrating the percentage of CD69⁺ and median fluorescent intensity (MFI) of CD25 staining on CD4⁺ and CD8⁺ T cells in activated human PBMCs treated with free EVs, conjugated EVs or free ligand, as determined by flow cytometry (n=3). FIG. 8(e) shows the schematic illustration of in vitro tumour killing assay (left panel) and the effect of free EVs, conjugated EVs or free ligands on the viability of H1975 cells based on a luciferase reporter for 48 hours (right panel), assessed using luciferase assay (n=4). ANOVA with Tukey's multiple comparisons: * P<0.05, ** P<0.01, *** P<0.001, and **** P<0.0001. ELISA: enzyme-linked immunosorbent assay, IFN-γ: interferon gamma, αPD-L1: anti-programmed death-ligand 1, αCD3: anti-cluster of differentiation 3, PBMCs: peripheral blood mononuclear cells.

FIG. 9 shows that bispecific EVs (BEVs) recruit activated T cells to tumour sites in a xenograft mouse model of lung cancer. FIG. 9(a) shows the experimental outline of the in vivo treatment employed to assess biodistribution of BEVs. H1975 cells bearing a luciferase reporter were used to monitor tumour inoculation. FIG. 9(b) shows representative IVIS fluorescent images of EV biodistribution in organs including liver (Li), lung (Lu), gastrointestinal tract (GI), spleen (Sp), kidney (Ki), and heart (H) from each treatment group including flowthrough (FT), free EV, EV-IgG, or BEV. EVs were labelled with DiR. FIG. 9(c) shows a bar graph demonstrating the summary of fluorescence intensity in different organs. The background fluorescence of each organ was subtracted (n=4 mice). FIG. 9(d) shows representative immunofluorescence images showing infiltration of CD8⁺ T cells into the tumour sites (indicated by dashed line) from each treatment group (Left). Tumour cells were stained with anti-luciferase antibody, CD8⁺ T cells were stained with anti-human CD8 antibody, and nuclei were stained with Hoechst. T, Tumour. Scale bar: 50 μm. Right: CD8⁺ T cell density (count/mm²) in tumour areas. FIG. 9(e) to FIG. 9(g) show bar graphs demonstrating the percentage of CD69⁺ cells in CD4⁺ and CD8⁺ T cells in the lung (FIG. 9(e)), spleen (FIG. 9(f)), and blood (FIG. 9(g)) from each treatment groups (n=4 mice). ANOVA with Tukey's multiple comparisons: ns—not significant, *P<0.05, ** P<0.01, *** P<0.001, and **** P<0.0001. IVIS: in vivo imaging system.

FIG. 10 shows that locally administered extracellular vesicles (EVs) are well retained in the immediate tumour microenvironment. FIG. 10(a) shows an illustration of intratracheal administration in mouse models. FIG. 10(b) shows representative near-IR fluorescent images of Free Ligand or EV-Ligand labels in organs acquired using IVIS 4 hours post-administration of a single intratracheal dose into C57BL/6 mice EV-Ligand or Free Ligand proteins were labelled with CF® Dye TFP Ester CF750, a near-IR amine reactive dye prior to administration to facilitate tracking of in vivo biodistribution. A flowthrough control is included for reference. FIG. 10(c) shows a bar graph demonstrating the summary of the in vivo biodistribution of Free Ligand and EV-Ligand treatments, represented as the relative percentage of the total administered dose present in each organ (n=3 mice per condition). FIG. 10(d) shows a line graph demonstrating the relative accumulation of Free Ligand and EV-Ligand treatments in the serum of the mice over a period of 42 hours, monitored via measurement of near-IR signals in the serum collected at each time interval. FIG. 10(e) shows box plots of blood chemistry analysis of mice administered with 6 doses of free ligand or an equivalent dose of EV-conjugated ligands, assessed 20 days after the first administration. Mice were administered the treatments intratracheally at 3-day intervals. FIG. 10(f) shows bar graphs demonstrating cellular uptake of control (EV/EV-IgG) or T-cell targeted EV-Ligand treatments by immune cell subsets in the lung assessed 5 hours after administration of a single dose of EVs. For FIG. 10(c) to FIG. 10(f), data was obtained from 3-4 independent repeats performed using EVs from individual donors. The ligand combination used in FIG. 10(b) to FIG. 10(f) consisted of αCD3, αCD137, αPD-1 and mIL-2. Student's one-tailed t-test: ns—not significant, *P<0.05, *** P<0.001. Error bars represent standard deviation. IVIS: in vivo imaging system.

FIG. 11 shows that multifunctional immunomodulatory EVs result in improved tumour suppression in a mouse model of lung metastatic melanoma. FIG. 11(a) shows the experimental outline of the in vivo treatment determined to assess the relative efficacy of EV-ligand compared to free ligands. B16 F20-Luc2 cells bearing a luciferase reporter were used to monitor tumour implantation and progression in the lung. FIG. 11(b) shows the representative IVIS images of mice from each treatment group. FIG. 11(c) shows a line graph of the luciferase signals from the lungs of mice administered with each of the indicated treatments over a 20-day period. FIG. 11(d) shows a line graph of the body weight of mice from each treatment group monitored over an extended period of time until the mice succumbed to the tumour. FIG. 11(e) shows a survival curve demonstrating survival of mice administered with each treatment described in FIG. 11(c). FIG. 11(f) shows immunofluorescent staining images of mouse lung sections from each treatment condition, assessing T cell infiltration and tumour burden. A combination of melanoma specific markers that are overexpressed in B16 F10-Luc2 cells was used to detect tumour cells (pan-cytokeratin, podoplannin, PD-L1 and firefly luciferase). Scale bar: 50 μm. FIG. 11(g) shows TUNEL staining image of lung sections from mice treated with EV-Ligand treatment. Scale bar: 50 μm. Figure (c) includes data from 5-6 mice per treatment condition. FIG. 11(d) to FIG. 11(e) include data from 5 mice for each condition. FIG. 11(h) shows a bar graph demonstrating the ALT levels of mice at the end of the treatment (Day 20) determined using a colorimetric assay for ALT activity. Two-way ANOVA test (c, d), Log-rank (Mantel-Cox) test (e) and Student's one-tailed t-test: * P<0.05, ** P<0.01, *** P<0.001. TUNEL: Terminal deoxynucleotidyl transferase dUTP nick end labelling.

FIG. 12 shows that surface of ligands synergizes with intraluminal loading of innate immune agonists. FIG. 12(a) shows bar graphs demonstrating the IL-12p70 levels in cell culture supernatants determined using ELISA following treatment of bone marrow-derived macrophages (BMDMs) or bone marrow derived dendritic cells (BMDCs) with free murine CD40 agonistic antibody or equivalent doses of EV-conjugated CD40 antibody. FIG. 12(b) shows graphs of median fluorescent intensity (MFI) of MHC II and costimulatory molecules on the surface of BMDCs following CD40 stimulation as in FIG. 12(a). FIG. 12(c) shows a bar graph demonstrating the IL-12p70 release in human monocyte-derived macrophages following stimulation with free agonistic human CD40 antibody or an equivalent dose of EV-conjugated antibodies. FIG. 12(d) shows immunofluorescence (IF) imaging of BMDMs incubated with control EVs, CD40-conjugated EVs or an equivalent dose of free CD40 antibody. Extracellular vesicles (EVs) were visualized using CellTrace Yellow labelling and cells were counterstained using Hoechst. Free CD40 antibody was visualised using a secondary anti-rat IgG. Cells were stained with a separate CD40 antibody that detected the cytoplasmic domain of CD40. Scale bar: 20 μm. FIG. 12(e) to FIG. 12(f) show graphs of median fluorescent intensity (MFI) of MHC II, CD80 and CD86 on the surface of BMDMs (FIG. 12(e)) or BMDCs (FIG. 12(f)) following stimulation with a fixed dose of free R848 or EV-loaded R848. EV-DMSO is included as a vehicle control. FIG. 12(g) shows bar graphs of IL12p70 release by BMDMs or BMDCs quantified using ELISA after 24 hours stimulation with R848 or EV-R848. (h) shows bar graphs demonstrating the fold change in IL-12p70 expression following treatment with either CD40-conjugated EVs, R848-loaded EVs or CD40-conjugated R848-loaded EVs. A cotreatment with equivalent doses of free CD40 antibody and R848 is included for comparison. For FIG. 12(a) to FIG. 12(c) and FIG. 12(e) to FIG. 12(h), data was obtained from 3-4 independent repeats performed using EVs from individual donors and macrophages/dendritic cells from different mice. Student's one-tailed t-test: ns—not significant, *P<0.05, *** P<0.001. Error bars represent standard deviation.

FIG. 13 shows that multifunctional immunomodulatory extracellular vesicles (EVs) result in enhanced tumour suppression in an autochthonous cell-derived model of PDAC. FIG. 13(a) shows a schematic of the in vivo experiment used to assess the anti-tumour efficacy of different treatments. Following intravenous (i.v.) injection of KPCY cells and subsequent intratracheal treatments, mice were sacrificed on day 30 to determine tumour burden, measure toxicity parameters, and perform immunophenotyping or alternatively maintained until they reached the criteria for symptom-free survival. In a separate experiment, lung-metastasis bearing mice were subcutaneously injected with 0.5 M KPCY cells to generate a secondary tumour challenge and the tumour burden was monitored for up to 20 days. FIG. 13(b) shows t-distributed stochastic neighbour embedding (tSNE) plots based on multicolour flow cytometric analysis illustrating the changes in immune cell composition in tumour-bearing mouse lungs on day 30. An opt-SNE algorithm was used for automated clustering and visualization of immune cell subsets and manually gated immune cell populations were overlaid on the tSNE plots after dimensionality reduction. Unidentified cells populations are left in grey colour and the proportion of tumour cells were included in the plot for reference (black). FIG. 13(c) shows a graph demonstrating the percentage of tumour cells in mouse lungs in each treatment group at the endpoint (Day 30). Tumour cells were gated using flow cytometry based on their expression of YFP. FIG. 13(d) to FIG. 13(e) show box plot demonstrating AST (FIG. 13(d)) and ALT (FIG. 13(e)) levels in the serum of mice on Day 30. FIG. 13(f) shows a line graph demonstrating the body weight of mice monitored over an extended period of time until the mice reached the criteria for symptom-free survival. FIG. 13(g) shows a survival curve demonstrating survival of mice administered with each treatment over a period of 60 days. FIG. 13(h) shows a line graph demonstrating the tumour volume of the flank tumour used to assess response to secondary tumour challenge. Mice were injected with tumour cells on day 15 and tumour volume monitored for 20 days post-implantation as shown in (a). Each line indicates 1 mouse. FIG. 13(c) to FIG. 13(h) include data from 5-7 mice per treatment condition. Two-way ANOVA test (FIG. 13(f)), Mixed effects analysis (FIG. 13(h)), Log-rank (Mantel-Cox) test (FIG. 13(g)) and Student's one-tailed t-test (FIG. 13(c) to FIG. 13(e)): * P<0.05, ** P<0.01, *** P<0.001. Error bars represent standard deviation.

FIG. 14 shows red blood cell-derived extracellular vesicle (RBCEV) purification and characterisation. FIG. 14(a) shows experimental schema used for the induction, isolation and purification of RBCEVs. FIG. 14(b) shows western blot characterisation of RBCEVs, comparing relative protein content compared to parental RBCs. FIG. 14(c) shows size distribution of unmodified RBCEVs determined using NTA. FIG. 14(d) shows the gating strategy used to gate out RBCEVs when performing single EV flow cytometric analysis. A buffer control and size standard are included for comparison. FIG. 14(e) shows flow cytometry dot plots from single EV flow cytometric characterisation of RBCEVs, staining for GPA, PS (using Annexin V) and CellTrace Far Red. RBCEVs: red blood cell-derived extracellular vesicle; NTA: nanoparticle tracking analysis, GPA: Glycophorin A, PS: phosphatidylserine. In (b), molecular weights of protein markers in kDa are shown on the left.

FIG. 15 shows optimisation and characterisation of ester-activated crosslinking and iEDDA reactions. FIG. 15(a) to FIG. 15(b) show western blot analysis of comparison of relative EV surface modification efficiency of STP ester compared to equal concentrations of NHS ester (FIG. 15(a)) or Maleimide (Mal) (FIG. 15(b)). Modification efficiency was assessed using reactive esters/maleimide carrying a biotin probe which was detected via western blot. FIG. 15(c) shows western blot analysis assessing the relative yield of Biotin-STP reacted with EV surface proteins under different pH or biotin-STP concentration. FIG. 15(d) shows flow cytometry dot plots depicting AZ488-TCO conjugation onto MTet-EVs acquired using a nanoflow cytometer. PBS, reagent controls and detergent controls are included to demonstrate the accuracy of EV detection and all samples were co-stained with CellTrace Far Red to more accurately identify EV populations. FIG. 15(e) shows a bar graph demonstrating the relative biotin signal of MTet-EVs sourced from individual blood donors that were reacted with Biotin-TCO. FIG. 15(f) shows a histogram from single EV flow cytometric analysis of Biotin-TCO conjugated EVs as compared to EVs directly reacted with Biotin-STP or control unconjugated EVs. FIG. 15(g) shows western blot demonstrating the relative stability of iEDDA-conjugated EVs over a period of 48 hours following incubation in human serum at 37 °C. NHS: N-Hydroxysuccinimide, STP: 4-Sulfo-2,3,5,6-tetrafluorophenyl. In FIG. 15(a), FIG. 15(b), FIG. 15(c), and FIG. 15(g), molecular weights of protein markers in kDa are shown on the left.

### DEFINITIONS

As used herein, the term “extracellular vesicles” refers to lipid bilayer-enclosed vesicles naturally released from cells.

As used herein, the term “red blood cell-derived extracellular vesicles” refers to vesicles produced by and/or obtained from red blood cells. In one example, the red blood cells are human red blood cells.

As used herein, the term “immunomodulatory molecules” refers to molecules that stimulates or suppresses the immune system. Examples of immunomodulatory molecules can be, but are not limited to, agonistic antibodies, antagonistic antibodies, cytokines, chemokines, small molecules, agonistic ligands, and combinations thereof.

As used herein, the term “small molecule” refers to any compound with a molecular weight of less than 1500 Da.

As used herein, the term “cis-conformation” refers to the orientation of ligands on the extracellular vesicles such that they are displayed on the surface of the same extracellular vesicle (EV) in relation to one another. In other words, in the context of EV conjugation referred to here, cis-conformation refers to two ligands being conjugated on the same EV as opposed to each ligand being conjugated on separate EVs. Thus, in cis-conformation, the 2 ligands are on a single EV in relation to one another, whereas in trans-conformation, they are on separate EVs.

As used herein, the term “crosslinking” refers to an active process of receptor clustering that occurs when higher order oligomers (multimerized) of ligands/antigen binding fragments/antibodies bind to multiple receptors on a single cell. Without being bound by theory, it is thought that this can result in greater receptor engagement, clustering and activation than native monomeric forms of the same ligand at equivalent doses.

As used herein, the term “nanoscale” refers to a nanoparticle with a size (diameter) of between 10-1000 nm. In some examples, the term nanoscale also refers to a particle size of between 50-400 nm. In another example, the extracellular vesicles disclosed herein are nanosized. In another example, the extracellular vesicles disclosed herein are between 50 to 400 nm in size.

As used herein, the term “extracellular vesicle surface functionalisation” refers to the process of modifying the outer surface of extracellular vesicles with various molecules or ligands to confer specific properties or functions. In some examples, the molecules or ligands can be, but are not limited to, agonistic antibodies, antagonistic antibodies, cytokines, chemokines, small molecules, agonistic ligands, and combinations thereof. In some examples, the molecules or ligands can be, but are not limited to, immunomodulatory molecules. In some examples, the immunomodulatory molecules can be, but are not limited to, agonistic antibodies, antagonistic antibodies, cytokines, chemokines, small molecules, agonistic ligands, and combinations thereof. In some examples, 2, 3, 4, 5, 6, 7, 8, 9 10, 11, 12, 13, 14, 15, 16, 17, 18, 19, 20, 21, 22, 23, 24, 25, 26, 27, 28 or more types of immunomodulatory molecules are anchored to the nanoscale extracellular vesicle.

As used herein, the term “Immunomodulatory EV (ImmEV)” or “EV-conjugated immunomodulatory ligands (EV-Ligand)” refers to nanoscale extracellular vesicles conjugated to molecules or ligands. The molecules or ligands can be, but are not limited to, immunomodulatory molecules. In some examples, the types of immunomodulatory molecules can be, but are not limited to, agonistic antibodies, antagonistic antibodies, cytokines, chemokines, small molecules, agonistic ligands, and combinations thereof.

As used herein, the term “bispecific extracellular vesicles (BEVs)” refers to nanoscale vesicles that have been modified to simultaneously display two different targeting ligands or molecules on their surface. These targeting ligands or molecules can bind to specific receptors or antigens present on target cells, enabling the selective recognition and binding of the modified EVs to multiple cell types or signalling pathways simultaneously.

As used herein, the term “agonistic” refers to molecules or ligands that bind to and activate receptors on cells to induce a biological reaction. In the present disclosure, reference is made to agonistic antibodies and agonistic ligands. For example, an agonistic ligand refers to a ligand that interacts with one or more receptors present on the surface of the target cell. As a person skilled in the art would appreciate that the term “antagonistic” is understood to refer to molecules with the opposite effect to agonistic molecules.

The following is a list of synonyms for the chemokines recited herein:

TABLE 1

| Chemokine | Synonym |
|---|---|
| CCL | MCP-1 |
| CCL3 | MIP-1a |
| CCL4 | MIP-1b |
| CCL5 | RANTES |
| CCL7 | MCP-3 |
| CCL11 | Eotaxin 1 |
| CCL13 | MCP-4 |
| CCL20 | MIP-3a |
| CCL22 | MDC |
| CCL24 | Eotaxin 2 |
| CCL26 | Eotaxin 3 |
| CCL28 | MEC |
| CXCL1 | GRO1 |
| CXCL8 | IL-8 |
| CXCL12 | SDF |
| CXCL13 | BCA-1 |
| XCL1 | Lymphotactin |
| CX3CL1 | Fractalkine |

### DETAILED DESCRIPTION OF THE PRESENT INVENTION

Immunotherapy has become an emerging strategy for the treatment of cancer. Delivery strategies and improved immunomodulators with higher specificity as to their target effect are under development. One example is the development of immunocytokines, which show an improved circulatory half-life and tumour retention compared to the half-life and tumour retention showed by free cytokines (which are cytokines without a tumour targeting moiety). This has been demonstrated in the example of Interleukin-2 (IL-2), where IL-2 fused to tumour targeting antibodies was shown to have an improved efficacy over free IL-2. There are also modified cytokines that are able to bind specifically to desired target cells to achieve desired therapeutic effects, a strategy particularly useful for pleiotropic cytokines. Bempegaldesleukin, a pegylated non-alpha IL-2 variant is an example of a pleiotropic cytokine engineered to bind preferentially to cytotoxic T cells, and not to regulatory T cells (Tregs), thereby promoting anti-tumour immune responses.

In addition to modifying the immunotherapeutic itself, modifications to delivery and administration strategies have also been taken into consideration. One example of this is that while high dose IL-2 can be toxic, and at times even fatal, the administration of low doses of IL-2 over a period of time has been shown to be safe, albeit less effective in terms of generating anti-tumour immunity.

There have also been efforts to improve the efficacy of immunotherapeutic molecules by attempting to reduce dosage and, consequently, toxicity. For example, the hepatotoxic effects associated with a CD137 agonistic antibody can be mitigated while preserving its therapeutic benefits by balancing its agonistic strength and its affinity for Fcγ receptors (FcγR).

Thus, disclosed herein is the use of the claimed extracellular vesicles (EVs) as delivery vectors that alter the biodistribution of a payload, for example, a cytokine, in the body of a subject, thereby limiting its off-target effects. For example, EVs engineered to display IL-12 on their surface were shown to have improved efficacy and reduced toxicity compared to an equivalent dose of free IL-12. Without being bound by theory, it was thought that both improved efficacy and reduced toxicity could be attributed to the alteration of biodistribution of the EVs, as the biodistribution of the EVs was limited to the immediate tumour microenvironment upon local administration. Thus, there was no systemic exposure of IL-12. Moreover, the EVs are able to maintain a high local concentration of IL-12 in the tumour, allowing improved immune activation in the tumour microenvironment.

The present disclosure describes a modular extracellular vesicle (EV)-based platform for enhancing the efficacy and safety of immunotherapy. As described herein, extracellular vesicles (EVs) were conjugated to multiple immunomodulatory molecules such as, but not limited to, antibodies, cytokines, chemokines, small molecules, agonistic ligands, and ligands (agonistic and otherwise). This strategy results in an increase immune activation than their free soluble forms at equivalent doses, thus resulting in improved immune activation and tumour suppression in vitro and in vivo. The EV-based platform of this disclosure is biocompatible and exhibits a favourable safety profile in preclinical mouse models.

In one example, disclosed herein is a nanoscale extracellular vesicle for immunotherapy, wherein one or more types of immunomodulatory molecules is/are anchored to the nanoscale extracellular vesicle. The types of immunomodulatory molecules anchored to the nanoscale extracellular vesicle can be, but are not limited to, agonistic ligands, antagonistic antibodies, cytokines, chemokines, small molecules, agonistic ligands, and combinations thereof.

Examples of agonistic antibodies as disclosed herein can be, but are not limited to, antibodies against cluster of differentiation 3 (anti-CD3), antibodies against cluster of differentiation 137 (anti-CD137), antibodies against cluster of differentiation 28 (anti-CD28), antibodies against cluster of differentiation 27 (anti-CD27), antibodies against OX-40 (anti-OX-40), antibodies against cluster of differentiation 357 (anti-CD357), antibodies against cluster of differentiation 40 (anti-CD40), antibodies against cluster of differentiation 278 (anti-CD278), and combinations thereof. In one example, the agonistic antibodies can be, but are not limited to, antibodies against cluster of differentiation 3 (anti-CD3), antibodies against cluster of differentiation 137 (anti-CD137), and antibodies against cluster of differentiation 28 (anti-CD28).

Examples of antagonistic antibodies as disclosed herein can be, but are not limited to, antibodies against programmed cell death protein 1 (anti-PD-1), antibodies against programmed death-ligand 1 (anti-PD-L1), antibodies against cytotoxic T-lymphocyte-associated protein 4 (anti-CTLA-4), antibodies against CD96 (anti-CD96), antibodies against T-cell immunoglobulin and mucin-domain containing-3 (anti-TIM-3), antibodies against lymphocyte-activation gene 3 (anti-LAG-3), and combinations thereof. In one example, the antagonistic antibodies can be, but are not limited to, antibodies against programmed cell death protein 1 (anti-PD-1) and antibodies against cytotoxic T-lymphocyte-associated protein 4 (anti-CTLA-4).

Examples of cytokines as disclosed herein can be, but are not limited to, interleukin-1 (IL-1), interleukin-2 (IL-2), interleukin-3 (IL-3), interleukin-4 (IL-4), interleukin-5 (IL-5), interleukin-6 (IL-6), interleukin-8 (IL-8), interleukin-9 (IL-9), interleukin-10 (IL-10), interleukin-11 (IL-11), interleukin-12 (IL-12), interleukin-13 (IL-13), interleukin-16 (IL-16), interleukin-17 (IL-17), interleukin-18 (IL-18), interleukin-23 (IL-23), interleukin-33 (IL-33), interleukin-1a (IL-1a), interleukin-13 (IL-13), tumour necrosis factor-alpha (TNF-α), tumour necrosis factor-beta (TNF-β), and combinations thereof. In one example, the cytokine is interleukin-2 (IL-2).

Examples of chemokines as disclosed herein can be, but are not limited to, C-C motif chemokine ligand 2 (CCL2), C-C motif chemokine ligand 3 (CCL3), C-C motif chemokine ligand 4 (CCL4), C-C motif chemokine ligand 5 (CCL5), C-C motif chemokine ligand 7 (CCL7), C-C motif chemokine ligand 8 (CCL8), C-C motif chemokine ligand 11 (CCL11), C-C motif chemokine ligand 13 (CCL13), C-C motif chemokine ligand 20 (CCL20), C-C motif chemokine ligand 22 (CCL22), C-C motif chemokine ligand 24 (CCL24), C-C motif chemokine ligand 26 (CCL26), C-C motif chemokine ligand 28 (CCL28), C-X-C motif chemokine ligand 1 (CXCL1), C-X-C motif chemokine ligand 8 (CXCL8), C-X-C motif chemokine ligand 12 (CXCL12), C-X-C motif chemokine ligand 13 (CXCL13), X-C motif chemokine ligand 1 (XCL1), CX3C motif chemokine ligand 1 (CX3CL1), and combinations thereof.

Examples of small molecules as disclosed herein can be, but are not limited to, Stimulator of Interferon Genes (STING) agonists, Toll-like receptor 7 (TLR7) agonists, Toll-like receptor 8 (TLR8) agonists, a dual toll-like receptor TLR7 and/or 8 (TLR7/8) agonists, and combinations thereof. In one example, the STING agonists as disclosed herein can be, but are not limited to, DMXAA (5,6-Dimethylxanthenone-4-acetic acid), cGAMP (Cyclic guanosine monophosphate-adenosine monophosphate), 2′3′-cGAMP (Cyclic dinucleotide), ADU-S100 (also known as MIW815 and ML RR-S2 CDA), MK-1454, SR-717, SB-11285, MK-2118, HG-381, BMS-986301, GSK3745417, E7766, SNX281, TAK-676, STG-982, STG-968, and combinations thereof. In one example, the TLR7 agonists as disclosed herein can be, but are not limited to, Guretolimod, Gardiquimod, Imiquimod, Loxoribine, GS-9620, 3M-011, 852A, CL264, TL7-887, TL7-975, and combinations thereof. In one example, the TLR8 agonists as disclosed herein can be, but are not limited to, Selgantolimod, VTX-2337, TL8-506, and combinations thereof. In one example, the TLR7/8 agonists as disclosed herein can be, but are not limited to, R848 (Resiquimod), MBS8, CL075, CL097, and combinations thereof.

Examples of agonistic ligands as disclosed herein can be, but are not limited to, Tumour Necrosis Factor (TNF), Lymphotoxin (LT), CD27 Ligand (CD27L), CD137 Ligand (CD137L), Glucocorticoid-Induced TNFR-Related Protein Ligand (GITRL), OX40 Ligand (OX40L), CD40 Ligand (CD40L), CD30 Ligand (CD30L), Tumour Necrosis Factor Superfamily Member 14 (TNFSF14), Lymphotoxin-alpha (LTα), B-cell Activating Factor (BAFF), A Proliferation-Inducing Ligand (APRIL), Tumour Necrosis Factor-Like Ligand 1A (TL1A), and combinations thereof.

In one example, the agonistic ligands as disclosed herein interact with their corresponding receptors to mediate immune responses. In another example, the agonistic ligands as disclosed herein interact with receptors from the Tumour Necrosis Factor Receptor Superfamily (TNFRSF). The following is a non-exhaustive list of exemplary agonistic ligands and their corresponding receptors (targets) in the Tumour Necrosis Factor Receptor Superfamily (TNFRSF):

TABLE 2

| | Tumour Necrosis Factor Receptor |
|---|---|
| Ligand | Superfamily (TNFRSF |
| Tumour Necrosis | TNF Receptor 1 (TNFR1) and TNF |
| Factor (TNF) | Receptor 2 (TNFR2) |
| Lymphotoxin (LT) | LTβ Receptor (LTβR) |
| CD27 Ligand | CD27 |
| (CD27L) | |
| CD137 Ligand | CD137 (also known as 4-1BB) |
| (CD137L) | |
| Glucocorticoid- | Glucocorticoid-Induced TNFR-Related |
| Induced TNFR- | Protein (GITR) and |
| Related Protein | Activation-Induced TNFR Family Receptor |
| Ligand (GITRL) | (AITR) |
| OX40 Ligand | OX40 (also known as CD134) |
| (OX40L) | |
| CD40 Ligand | CD40 |
| (CD40L) | |
| CD30 Ligand | CD30 |
| (CD30L) | |
| Tumour Necrosis | Herpesvirus Entry Mediator (HVEM) and |
| Factor | Lymphotoxin beta receptor (LTβR) |
| Superfamily | |
| Member 14 | |
| (TNFSF14) | |
| Lymphotoxin- | Herpesvirus Entry Mediator (HVEM) and |
| alpha (LTα) | Lymphotoxin beta receptor (LTβR) |
| B-cell Activating | BAFF Receptor (BAFF-R) |
| Factor (BAFF) | |
| A Proliferation- | Transmembrane Activator and Calcium- |
| Inducing Ligand | Modulating Cyclophilin Ligand Interactor |
| (APRIL) | (TACI) and B-cell Maturation Antigen |
| | (BCMA, also known as TNFRSF17) |
| Tumour Necrosis | Death Receptor 3 (DR3, also known as |
| Factor-Like | TNFRSF25) |
| Ligand 1A | |
| (TL1A) | |

Examples of tumour necrosis factor receptors as disclosed herein (which function as targets of the ligands and/or the antibodies disclosed herein) can be, but are not limited to, Tumour Necrosis Factor Receptor 1 (TNFR1), Tumour Necrosis Factor Receptor 2 (TNFR2), Lymphotoxin Beta Receptor (LTBR), Cluster of Differentiation 27 (CD27), Cluster of Differentiation 137 (CD137), Glucocorticoid-Induced TNFR-Related protein (GITR), OX40, CD40, CD30, Herpesvirus Entry Mediator (HVEM), B-Cell Activating Factor Receptor (BAFFR), Activation-Induced TNFR Family Receptor (AITR), Death Receptor 3 (DR3), Transmembrane Activator and CAML Interactor (TACI), and combinations thereof.

In one example, the immunomodulatory molecules are recombinant molecules. In one further example, the cytokines disclosed herein are of recombinant origin. In another example, the choice of which immunomodulatory molecules are anchored on the EVs is dependent on the target cell to which the surface modified EVs are to bind to. In one example, a receptor on the target cell can be targeted by two or more immunomodulatory molecules. For example, both an agonistic ligand and an agonistic antibody can be used to target the same receptor on the target cell.

As disclosed herein, the extracellular vesicles can originate from sources such as, but not limited to, a cell or a compound capable of forming extracellular vesicles. In one example, the cell capable of forming extracellular vesicles can be, but not limited to, a red blood cell and a cancer cell. In one example, the cell capable of forming extracellular vesicles is a red blood cell. In another example, the compound capable of forming extracellular vesicles can be, but not limited to, milk and a plasmid.

In one example, the extracellular vesicles disclosed herein can be derived from, but are not limited to, human red blood cell-derived extracellular vesicles (RBCEVs). As compared to synthetic formulations or immortalised cell-derived extracellular vesicles, RBCEVs have been shown to be safe, scalable, and biocompatible as a drug delivery vector, thereby showing the efficacy and safety of the extracellular vesicles as delivery vectors of immunotherapeutics. As disclosed herein, the extracellular vesicles can be nanoscale extracellular vesicles released by red blood cells (RBCs). In such an example, the constituent components and biomolecules of the EVs are exclusively derived from RBCs. Moreover, RBCEVs, as drug delivery vectors, have been demonstrated herein to be efficient at delivering a range of encapsulated therapeutics to cancer cells in vitro and in vivo.

The extracellular vesicles, as disclosed herein, can be applied as, for example, delivery vectors. The effect of these extracellular vesicles makes use of the fact that locally administered nanoparticles would remain in the immediate tumour microenvironment, thereby limiting systemic toxicity and facilitating potent local immune activation and anti-tumour immunity.

In one example, disclosed herein is an immunomodulatory EV-based platform for cancer treatment that comprises EVs conjugated to single immunomodulatory molecule or EVs conjugated to multiple immunomodulatory molecules; wherein the EV-conjugated ligands confer functionality and safety and biodistribution profiles to conjugated ligands. In another example, the EVs disclosed herein are membrane vesicles isolated from any cell types. In yet another example, the EVs disclosed herein are non-exosomal EVs isolated from RBCs. In one example, the immunomodulatory molecules include, but are not limited to, antibodies, cytokines, chemokines, small molecules, agonistic ligands, and ligands.

Monotherapy with immunotherapeutics have been shown to be rarely effective due to the complex nature of most tumours. The tumour microenvironment is complex, with many immune checkpoints and immune suppressive factors present. To improve the effectiveness of immunotherapeutics in the tumour microenvironment, a combination of immunomodulators has been identified that work complementarily to target T-cells and induce immune activation and anti-tumour effects. A combination of ligands to target multiple aspects of a mouse melanoma model is also encompassed within in the scope of this disclosure. The present disclosure also demonstrates that EV-ligand treatments showed improved treatment effects and an enhanced safety profile in mice, accompanied by an increase in survival rate, compared to free ligands.

Therefore, in one example, the nanoscale extracellular vesicle is as disclosed herein, wherein 2, 3, 4, 5, 6, 7, 8, 9 10, 11, 12, 13, 14, 15, 16, 17, 18, 19, 20, 21, 22, 23, 24, 25, 26, 27, 28 or more types of immunomodulatory molecules are anchored to the nanoscale extracellular vesicle. In one example, disclosed herein is the conjugation of multiple ligands on the surface of EVs to improve their targeting specificity and modulate the tumour microenvironment. The multiple ligands that can be conjugated on the EV surface include, but are not limited to, antibodies, cytokines, chemokines, small molecules, agonistic ligands, and ligands.

In another example, disclosed herein is a bispecific nanoscale extracellular vesicle. Such the bispecific nanoscale extracellular vesicle results in bridging T cells and tumour cells. In another example, the present disclosure describes bispecific EVs conjugated with both anti-CD3 and anti-PD1 antibodies. The use of two antibodies in this example allows the binding of the bispecific EVs to both T cells and tumour cells to activate and recruit the T cells to the tumour sites.

Extracellular vesicles are known to be readily taken up by phagocytic cells such as macrophages and dendritic cells. These phagocytic cells express CD40, crosslinking-dependent member of the Tumour Necrosis Factor Receptor Superfamily (TNFRSF), which plays a role in modulating anti-tumour immune responses and is a target for immunotherapy. Therefore, also disclosed herein is a nanoscale extracellular vesicle further comprises a payload, wherein the payload is loaded into the lumen of the extracellular vesicle. In one example, the payload as disclosed herein can be, but not limited to, small molecules, synthetic DNA molecules, and synthetic RNA molecules.

In one example, the small molecules, synthetic DNA molecules, and synthetic RNA molecules as disclosed herein can enhance immune responses against cancer. Examples of small molecules can be, but not limited to, Stimulator of Interferon Genes (STING) agonists, Toll-like receptor 7 (TLR7) agonists, Toll-like receptor 8 (TLR8) agonists, a dual toll-like receptor TLR7 and/or 8 (TLR7/8) agonists, and combinations thereof. In one example, the STING agonists as disclosed herein can be, but are not limited to, DMXAA (5,6-Dimethylxanthenone-4-acetic acid), cGAMP (Cyclic guanosine monophosphate-adenosine monophosphate), 2′3′-cGAMP (Cyclic dinucleotide), ADU-S100 (also known as MIW815 and ML RR-S2 CDA), MK-1454, SR-717, SB-11285, MK-2118, HG-381, BMS-986301, GSK3745417, E7766, SNX281, TAK-676, STG-982, STG-968, and combinations thereof. In one example, the TLR7 agonists as disclosed herein can be, but are not limited to, Guretolimod, Gardiquimod, Imiquimod, Loxoribine, GS-9620, 3M-011, 852A, CL264, TL7-887, TL7-975, and combinations thereof. In one example, the TLR8 agonists as disclosed herein can be, but are not limited to, Selgantolimod, VTX-2337, TL8-506, and combinations thereof. In one example, the TLR7/8 agonists as disclosed herein can be, but are not limited to, R848 (Resiquimod), MBS8, CL075, CL097, and combinations thereof. Examples of synthetic DNA molecules can be, but are not limited to, CpG ODNs (CpG oligodeoxynucleotides) and oligonucleotides that target toll-like receptors, as well as the retinoic acid-inducible gene I (RIG-I), and melanoma differentiation-associated protein 5 (MDA5). In one example, the payload is R848 (Resiquimod).

Some examples of the chemical structure of the small molecules as disclosed herein include, but are not limited to, the chemical structures shown below:

| Small molecule | Chemical structure |
|---|---|
| R848 (Resiquimod) | 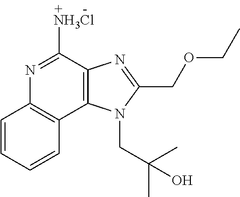 |
| DMXAA (5,6- Dimethyl- xanthenone- 4-acetic acid) | 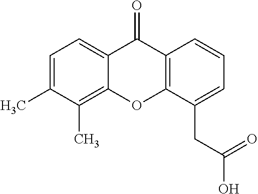 [SMILES: Cc1ccc2c(=O)c3cccc(CC(=O)O)c3oc2c1C] |
| ADU-S100 | 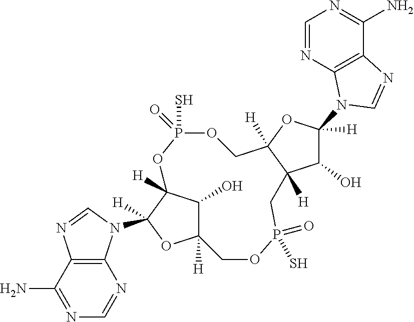 [SMILES: Nc1ncnc2c1ncn2[C@@H]1O[C@@H]2CO[P@@](=O)(S)O[C@@H]3[C@H](O)[C@@H](CO[P@@](=O)(S)C[C@H]2[C@H]1O)O[C@H]3n1cnc2c(N)ncnc21] |
| STG-982 | 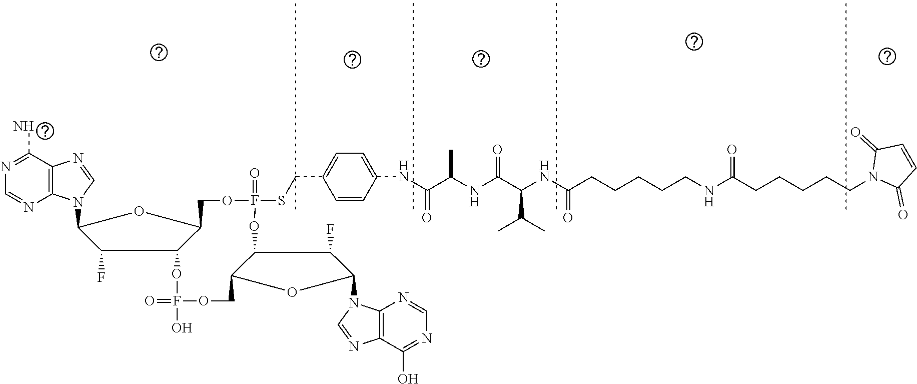 |
| STG-968 | 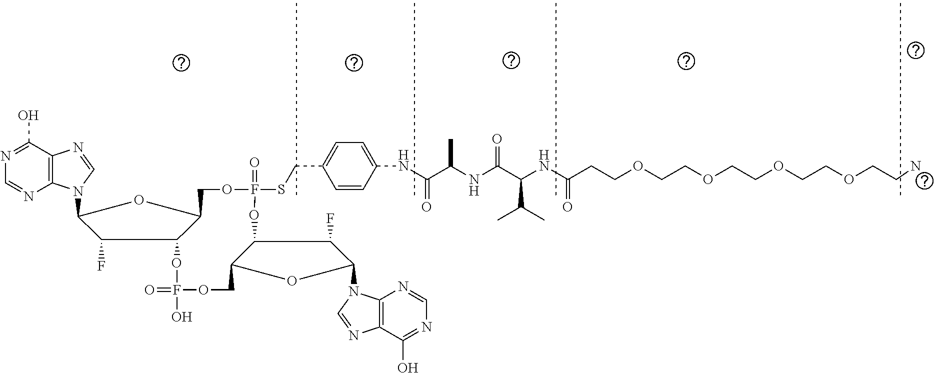 |
| TL7-887 | 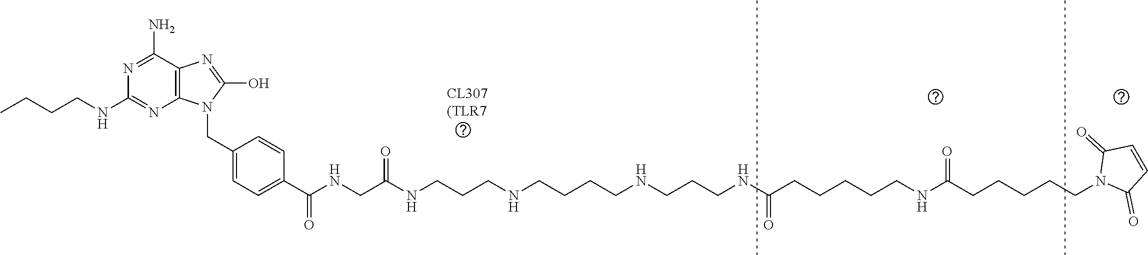 [SMILES: CCCCNc1nc(N)c2nc(O)n(Cc3ccc(C(=O)NCC(=O)NCCCNCCCCNCCCNC(=O)CCCCCNC(=O)CCCCCN4C(=O)C=CC4=O)cc3)c2n1] |
| TL7-975 | 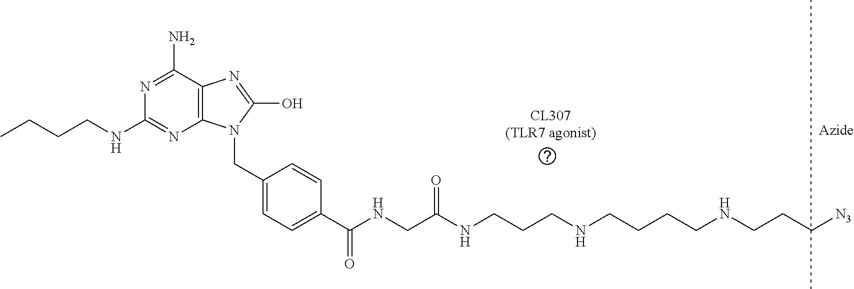 [SMILES: CCCCNc1nc(N)c2nc(O)n(Cc3ccc(C(=O)NCC(=O)NCCCNCCCCNCCCN=[N+]=[N-])cc3)c2n1] |
| Muramyl dipeptide | 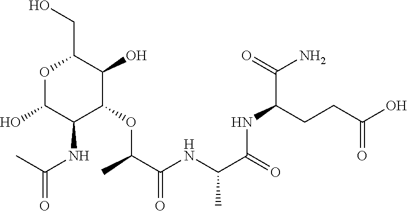 [SMILES: CC(=O)N[C@@H]1[C@@H](O[C@H](C)C(=O)N[C@@H](C)C(=O)N[C@H](CCC(=O)O)C(N)=O)[C@H](O)[C@@H](CO)O[C@H]1O] |
| Imiquimod | 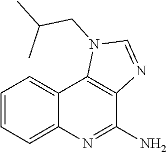 [SMILES: CC(C)Cn1cnc2c(N)nc3ccccc3c21] |

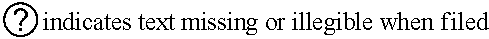

Disclosed herein are methods for loading extracellular vesicles with payloads. In one example, methods disclosed herein are for loading payloads into the lumen of extracellular vesicles. In one example, the payload can be loaded into the extracellular vesicle lumen via, but are not limited to, permeabilisation of the extracellular vesicle membrane. In one example, permeabilisation of the extracellular vesicle membrane can be achieved through methods including, but not limited to, chemical permeabilisation, physical permeabilisation, enzymatic permeabilization, electroporation, and combinations thereof. In one example, the chemical agents used for permeabilising the extracellular vesicle membrane can be, but are not limited to, Dimethyl sulfoxide (DMSO), Triton X-100, saponin, and combinations thereof. In another example, the chemical agents used for permeabilising the extracellular vesicle membrane is DMSO.

The nanoscale extracellular vesicle as disclosed herein for use in therapy. In one example, the therapy is an immunotherapy. In another example, the nanoscale extracellular vesicle as disclosed herein is for treating or preventing a disease. In yet another example, the nanoscale extracellular vesicle as disclosed herein is for treating or preventing cancer.

The nanoscale extracellular vesicle as disclosed herein can be formulated into compositions suitable for administration. Where applicable, the nanoscale extracellular vesicle as disclosed herein may be administered with a pharmaceutically acceptable carrier. A “carrier” can include any pharmaceutically acceptable carrier, as long as the carrier is compatible with other ingredients of the formulation and not injurious to the subject. Accordingly, pharmaceutical compositions for use can be formulated in conventional manner using one or more physiologically acceptable carriers comprising excipients and auxiliaries which facilitate processing of the active compounds into preparations which can be used pharmaceutically. Proper formulation is dependent upon the route of administration chosen.

In one example, the present disclosure describes a method of treating or preventing a disease, the method comprising administering a composition comprising one or more nanoscale extracellular vesicles as disclosed herein to a subject. In one example, the composition comprising one or more nanoscale extracellular vesicles as disclosed herein can be administered to a subject through various routes including, but not limited to, intravenous (IV) injection, intraperitoneal (IP) injection, subcutaneous (SC) injection, intramuscular (IM) injection, intratracheal or intranasal administration, and combinations thereof.

In yet another example, the nanoscale extracellular vesicle as disclosed herein can be formulated into compositions suitable for administration, wherein the composition further comprises one or more pharmaceutically acceptable excipients, vehicles or carriers.

In one example, the disease to be treated or prevented is cancer. In this example, the immunomodulatory molecules are molecules suitable or able to treat or prevent cancer. Cancers can be defined either by their location within a host or by the type of cell that is cancerous. Examples of the former are, but are not limited to, breast cancer, lung cancer, pancreatic cancer. By way of an example for the latter (cancers defined by cell type), a carcinoma is a type of cancer that forms in epithelial tissue, whereas a melanoma is a type of cancer that forms in melanocytes.

In one example, the cancer to be treated or prevented can be, but are not limited to, lung adenocarcinoma, breast adenocarcinoma, prostate adenocarcinoma, colorectal adenocarcinoma, bladder urothelial carcinoma, ureter urothelial carcinoma, renal pelvis urothelial carcinoma, bladder squamous cell carcinoma, ureter squamous cell carcinoma, renal pelvis squamous cell carcinoma, skin squamous cell carcinoma, lung squamous cell carcinoma, bladder transitional cell carcinoma, ureter transitional cell carcinoma, renal pelvis transitional cell carcinoma, bladder leiomyosarcoma, ureter leiomyosarcoma, renal pelvis leiomyosarcoma, bladder rhabdomyosarcoma, ureter rhabdomyosarcoma, renal pelvis rhabdomyosarcoma, breast angiosarcoma, prostate angiosarcoma, colorectal angiosarcoma, bladder fibrosarcoma, ureter fibrosarcoma, renal pelvis fibrosarcoma, bladder acute lymphocytic leukaemia (ALL), ureter acute lymphocytic leukaemia (ALL), renal pelvis acute lymphocytic leukaemia (ALL), bladder acute myeloid leukaemia (AML), ureter acute myeloid leukaemia (AML), renal pelvis acute myeloid leukaemia (AML), bladder chronic lymphocytic leukaemia (CLL), ureter chronic lymphocytic leukaemia (CLL), renal pelvis chronic lymphocytic leukaemia (CLL), bladder chronic myeloid leukaemia (CML), ureter chronic myeloid leukaemia (CML), renal pelvis chronic myeloid leukaemia (CML), Hodgkin lymphoma of the bladder, non-Hodgkin lymphoma of the bladder, multiple myeloma of the bladder, bladder gliomas (astrocytoma, glioblastoma), ureter gliomas (astrocytoma, glioblastoma), renal pelvis gliomas (astrocytoma, glioblastoma), bladder meningiomas, ureter meningiomas, renal pelvis meningiomas, bladder medulloblastoma, ureter medulloblastoma, renal pelvis medulloblastoma, bladder ependymoma, ureter ependymoma, renal pelvis ependymoma, bladder oligodendroglioma, ureter oligodendroglioma, renal pelvis oligodendroglioma, breast cancer, prostate cancer, lung cancer (small cell, non-small cell), colorectal cancer, pancreatic cancer, liver cancer, stomach cancer, kidney cancer (renal cell carcinoma, Wilms tumour), bladder cancer, oesophageal cancer, ovarian cancer, cervical cancer, uterine cancer, endometrial cancer, testicular cancer, penile cancer, vaginal cancer, vulvar cancer, thyroid cancer, head and neck cancer (oral, nasopharyngeal, laryngeal), melanoma skin cancer, non-melanoma skin cancer (basal cell carcinoma, squamous cell carcinoma), gallbladder cancer, bile duct cancer (cholangiocarcinoma), adrenal cancer, and mesothelioma. In another example, the cancer to be treated or prevented can be, but are not limited to, metastatic cancer. In one example, the melanoma skin cancer is lung metastatic melanoma. In another example, the pancreatic cancer is pancreatic ductal adenocarcinoma.

Compositions and formulations for intravenous, parenteral, intrathecal or intraventricular administration can include sterile aqueous solutions that can also contain buffers, diluents and other suitable additives such as, but not limited to, penetration enhancers, carrier compounds and other pharmaceutically acceptable carriers or excipients. In one example, the pharmaceutical composition described herein is formulated for intravenous administration. In another example, the pharmaceutical composition is formulated as admixture, whereby, for example, each component is provided separately, to be mixed shortly before administration.

The formulations as described herein, which can conveniently be presented in unit dosage form, can be prepared according to conventional techniques well known in the pharmaceutical industry. Such techniques include, for example, the step of bringing into association the extracellular vesicles disclosed herein with the pharmaceutical carrier(s) or excipient(s).

Further envisioned in the scope of the present disclose is a kit comprising the nanoscale extracellular vesicle as disclosed herein.

The immunomodulatory EV-based platform disclosed herein presents multiple advantages over other methods as it improves the efficacy and safety of immunotherapeutics, resulting in potent local immune activation and anti-tumour immunity. The advantages are at least as follows:

(i) RBCEVs are from human red blood cells, which makes them more biocompatible than other drug delivery vectors, such as synthetic nanoformulations or immortalized cell derived EVs. Furthermore, RBCEVs lack oncogenic DNA/RNA and growth factors commonly found in EVs derived from cancer cells or stem cells, thus avoiding transformation risks to recipient cells. Additionally, compared to known drug delivery methods such as viruses or synthetic transfection reagents, RBCEVs are non-immunogenic and matched to blood groups, offering high specificity with minimal systemic toxicity. (ii) Conjugation of immunomodulatory molecules onto RCBEVs enhances their ability to increase immune activation and anti-tumour responses as compared to equivalent doses of free soluble immunomodulatory molecules. This is thought to be the result of EV-mediated multimerization of ligands and receptor crosslinking. RBCEVs can be adapted to deliver multiple immunomodulatory molecules for the treatment of various cancers, and hence further facilitating the development of personalised medicine. (iii) The iEDDA (Inverse electron-demand Diels-Alder)-mediated conjugation approach for surface functionalisation of EVs is stable, efficient, translatable to any protein of interest and resulted in copy numbers far exceeding any previously reported in the field of EV engineering. (iv) Immunomodulatory RBCEV-based platform can be locally administered and have been shown to remain in the immediate tumour microenvironment, thereby limiting systemic toxicity. (v) The increased local retention (of the EVs to the diseased site, for example) also facilitates maintenance of high concentration of therapeutics in the tumour microenvironment, thereby resulting in increased anti-tumour responses at lower doses. (vi) The decreased systemic exposure and lower doses administered allow the use of more complex combinations of cytokines, the use of which would otherwise be negated due to the onset of adverse side-effects. Immunomodulatory RBCEV-based platform utilises the EV surface to multimerize antibodies, resulting in increased receptor crosslinking and the formation of immune synapses upon EV binding and endocytosis, facilitating an increase immune activation than would be achievable with an equivalent dose of free ligands. Furthermore, as described herein, the RBCEV-based platform can deliver a combination of complementary immunomodulatory molecules designed to target multiple aspects of the anti-tumour response within the complex tumour microenvironment, leading to effective killing of tumour cells and enhancement in survival in murine melanoma allografted mice. In one example, using the method disclosed herein, multiple immunomodulatory molecules can be conjugated at different concentrations to the surface of the RBCEVs by altering the ratio of incubated immunomodulatory molecules.

EVs can be Efficiently Functionalised Using iEDDA-Mediated Conjugation

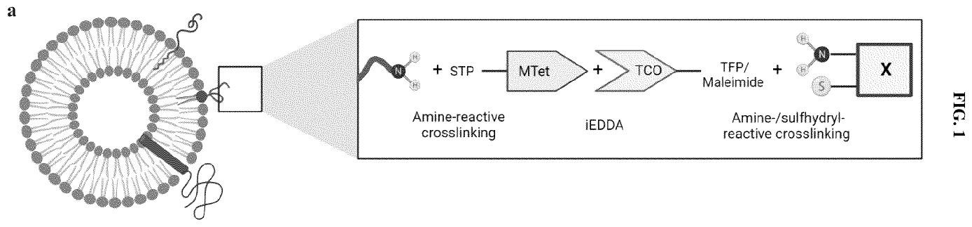

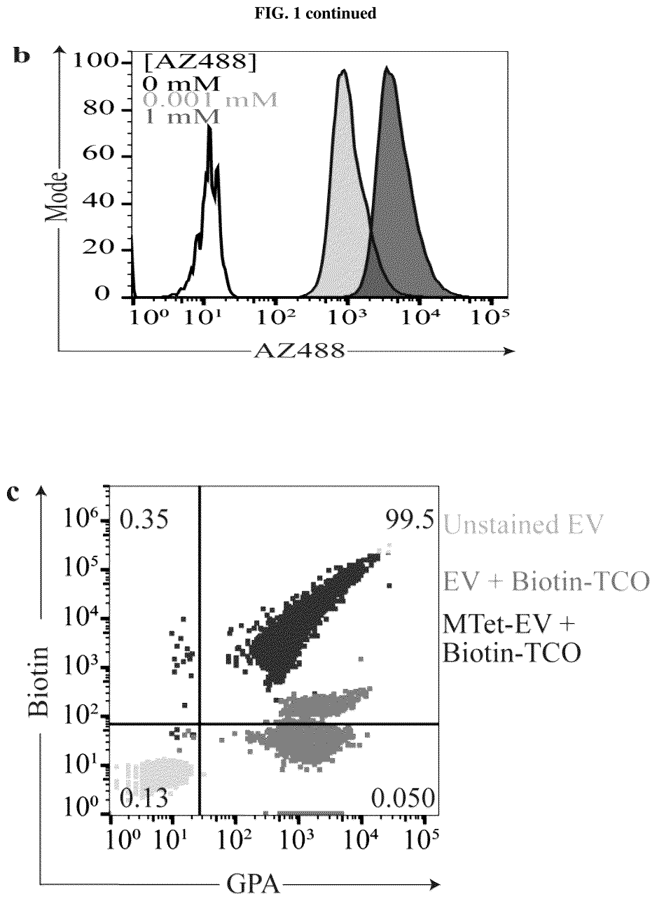

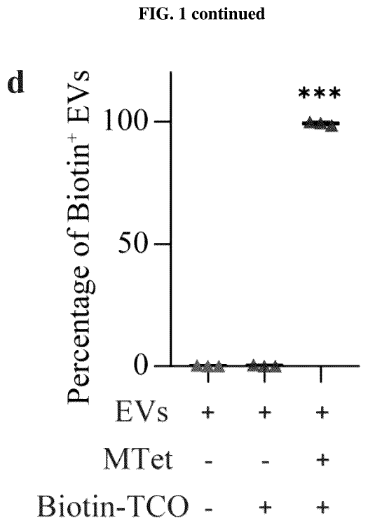

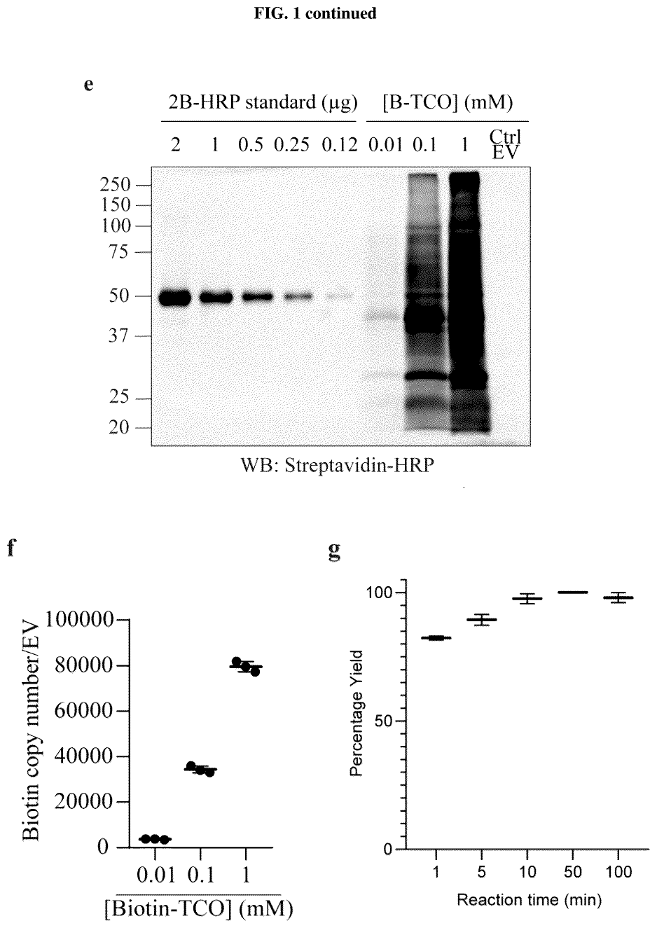

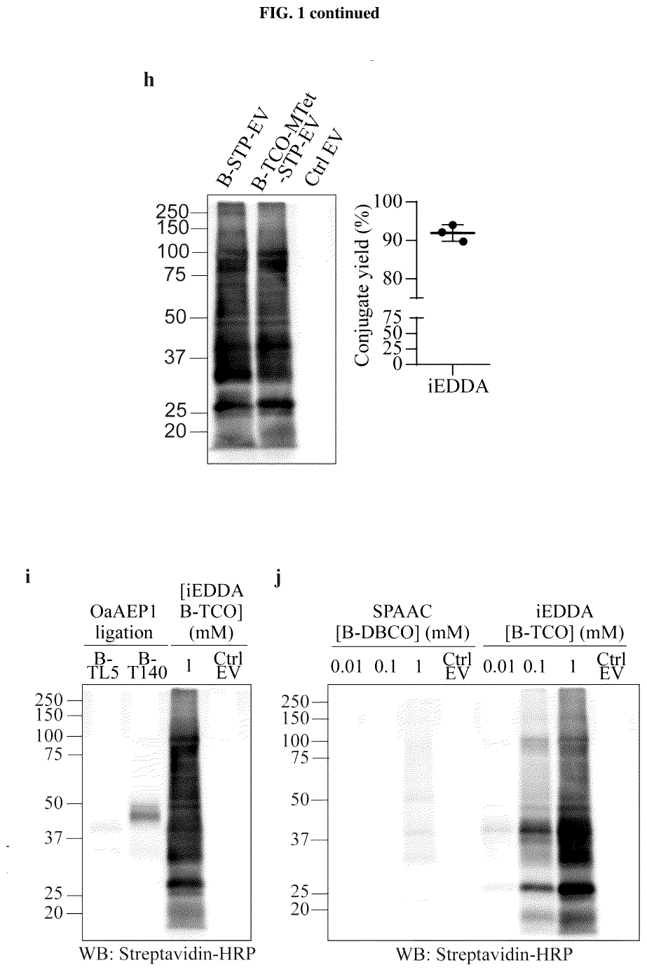

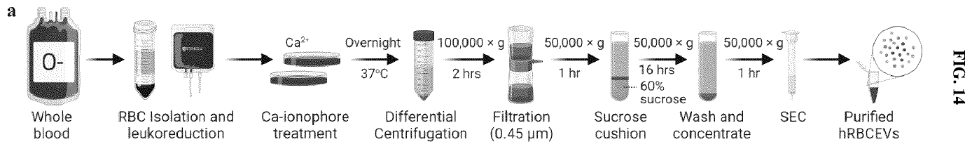

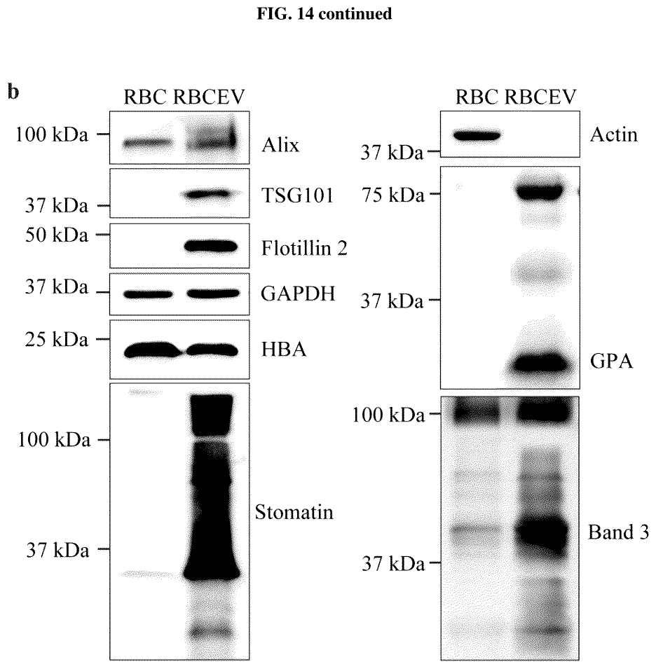

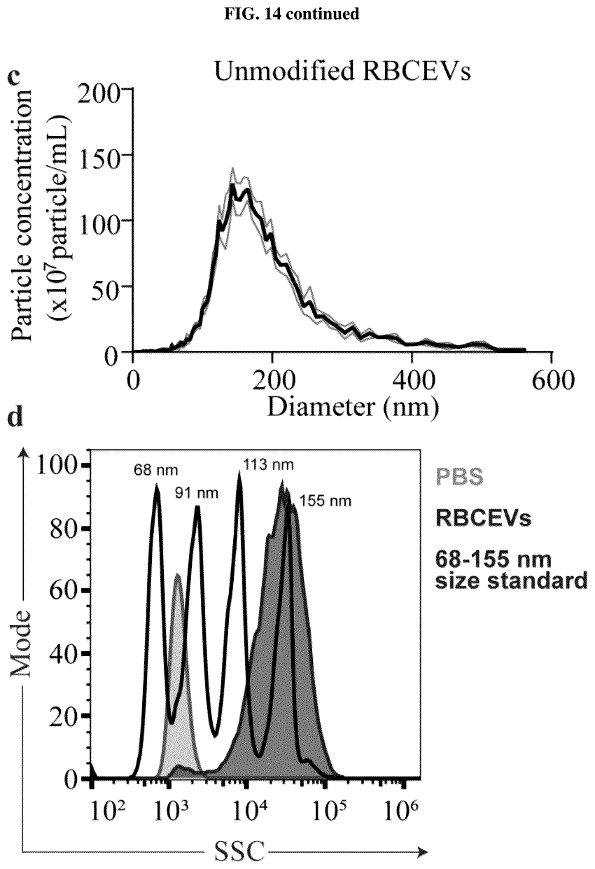

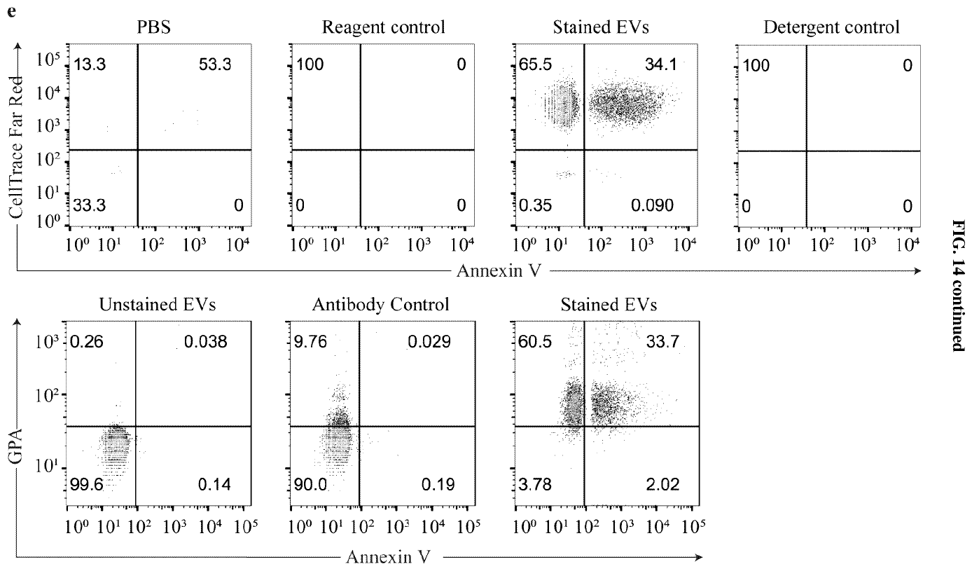

RBCEVs were isolated and purified from human red blood cells as outlined in FIG. 14(a). Western blot analysis of bulk RBCEVs in comparison to equivalent quantified of cell lysate revealed the enrichment of EV specific markers (Alix, TSG101, Flotillin 2) and RBC membrane proteins (Stomatin, Glycophorin A, Band 3). The depletion of cytoskeletal actin was also observed, while the level of other cytosolic proteins such as Haemoglobin and GAPDH remained the same (FIG. 14(b)). Nanoparticle tracking analysis revealed that the bulk of the RBCEVs had a size distribution ranging from 50 nm-250 nm with an average diameter of ~160 nm (FIG. 14(c)). Single EV flow cytometric analysis of RBCEVs was also performed using a NanoFCM system. RBCEVs were gated from background as shown in FIG. 1D using SSC channel (FIG. 14(d)). Size standard beads and a PBS control are shown for comparison. EVs were characterized by staining for the RBC-specific surface protein glycophorin A (GPA), phosphatidylserine (PS) exposure and CellTrace Far Red staining. It was shown that the majority of EVs gated based on SSC were positive for GPA and stained positive for CellTrace as evidenced by the upward shift in the entire EV population. However, two distinct populations of EVs were observed based on PS exposure—about 34% of EVs were PS positive and formed a distinct Annexin V^{high} population while the remaining EVs showed only a moderate shift indicating significantly lower levels of PS exposure. Importantly, events detected were EVs as upon the addition of Triton X-100, all the EV events in the stained sample disappeared, confirming that they were contributed by lipid-based vesicles.

In one example, disclosed herein is a nanoscale extracellular vesicle for immunotherapy, wherein the size of the nanoscale extracellular vesicle ranges between 1 to 1000 nm. In another example, the size of the nanoscale extracellular vesicle disclosed herein ranges between 10 to 1000 nm. In another example, the size of the nanoscale extracellular vesicle disclosed herein ranges between 50 to 400 nm. In another example, the size of the nanoscale extracellular vesicle disclosed herein ranges between 50 to 250 nm. In another example, the size of the nanoscale extracellular vesicle disclosed herein is about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, and about 250 nm. In another example, the size of the nanoscale extracellular vesicle disclosed herein is about 160 nm.

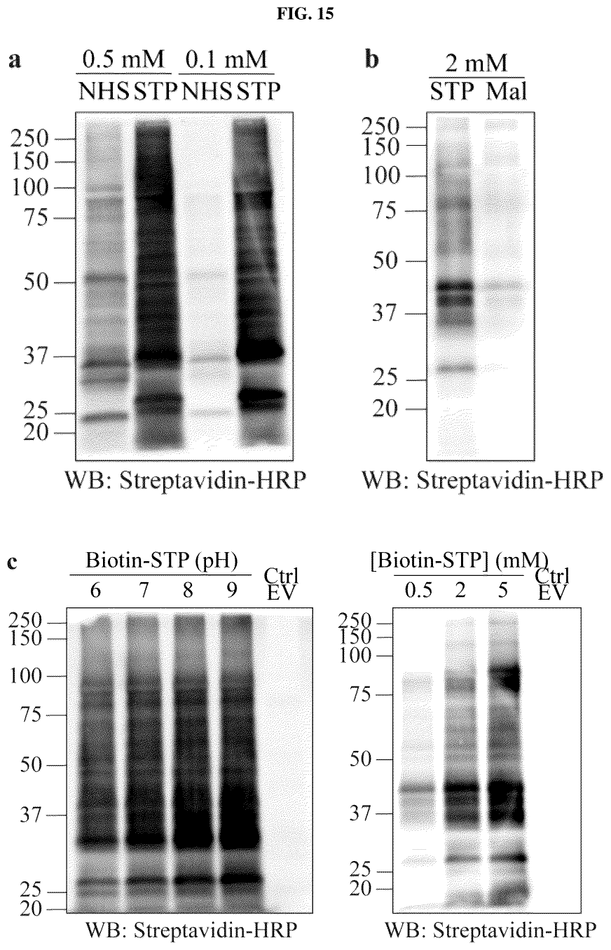

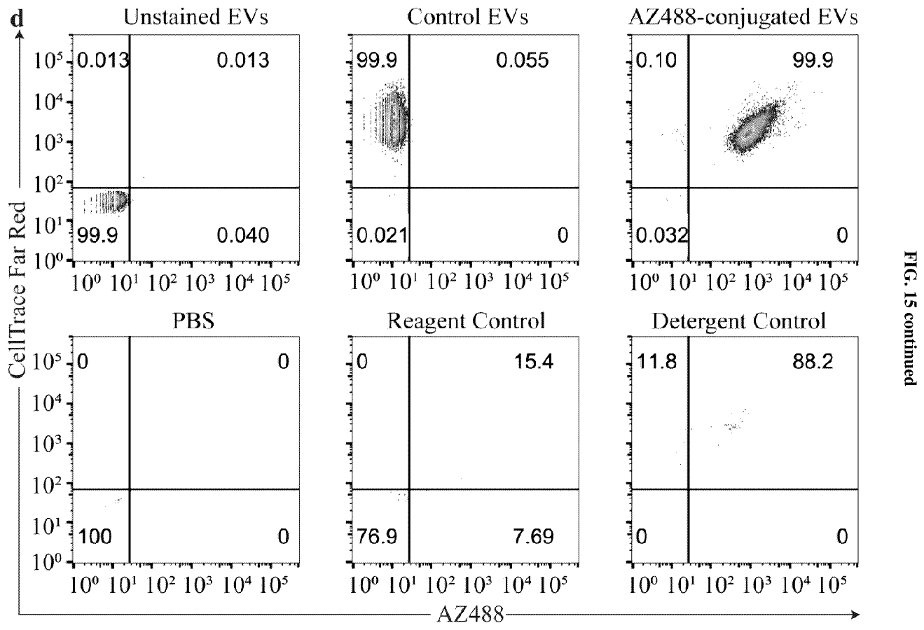

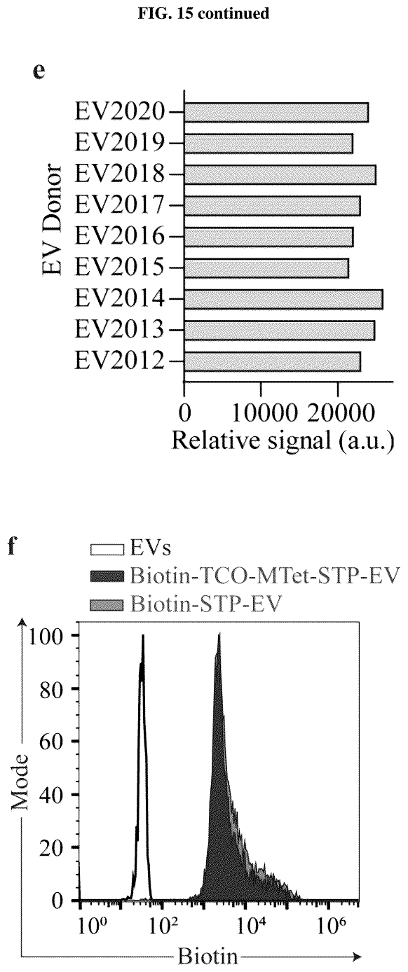

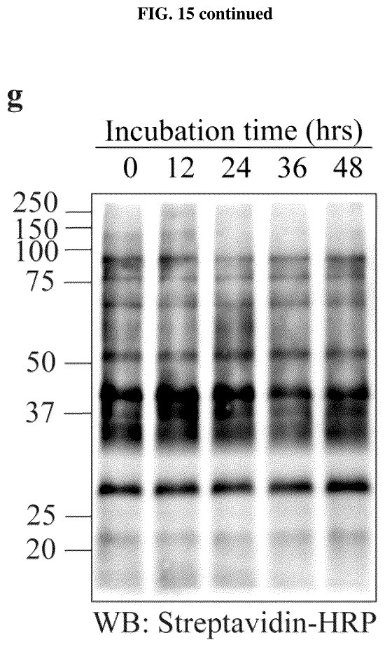

To modify the surface of the EVs to install the click handles for iEDDA, the relative efficacy of amine reactive esters and sulfhydryl reactive chemistry were investigated. Ester crosslinking involves the use of N-hydroxysuccinimide (NHS) esters or reactive esters of a similar nature that are reactive towards primary amine groups that are found abundantly on the N-terminal of all proteins and on lysine side chains. As disclosed herein, the relative efficiency of conventional NHS esters was compared with 4-Sulfo-2,3,5,6-tetrafluorophenyl (STP) esters, a newer amine reactive ester that is completely soluble in aqueous media and shows improved stability and reactivity as compared to NHS esters. To this end, RBCEVs were reacted with each ester carrying a biotin probe under similar reaction conditions and the reaction yield assessed using western blot, probing for biotin using streptavidin-HRP. Given the non-specific nature of the conjugation approach that would be expected to conjugate biotin on any proteins on the EV surface with a protonated primary amine group, a smeared banding pattern was observed. Initial analysis revealed that the STP ester resulted in higher copy numbers as evidenced by the more intense banding pattern in FIG. 15(a). It was thus decided to utilize STP esters as an alternative to NHS esters. The efficiency of STP esters to Maleimide conjugation was also compared. Maleimide crosslinking relies on the formation of stable thioether linkages between the maleimide group and free reduces S groups that are commonly found on Cysteine side chains. Reaction under similar conditions revealed that STP-mediated crosslinking was significantly more efficient that maleimide crosslinking FIG. 15(b). Thus, subsequent crosslinking experiments on EV surface functionalisation were conducted using STP esters. A series of experiments to optimize the pH and STP concentration to obtain optimal yield were performed. As shown in FIG. 15(c). Optimal yield with STP esters was obtained at higher pH values (pH 9) and higher STP concentrations (5 mM). For the sake of maintaining a balance between functional efficacy and biocompatibility, an STP concentration of 2 mM and a pH of 8.5 were used for subsequent experiments.

FIG. 1(a) outlines the overall outline of the conjugation approach utilized for iEDDA-mediated conjugation of EVs. In brief, Methyltetrazine (MTet)-STP was used to install the MTet click handle on the EV surface using the STP reaction conditions optimized above. Similarly, a complementary transcyclooctene (TCO) click handle was installed on a desired protein X using tetrafluorophenyl (TFP) esters or Maleimide crosslinkers. The MTet-EVs and the TCO-Protein X were subsequently purified to remove unreacted click handles and incubated together to facilitate the formation of covalent bonds between the EV and the protein of interest via iEDDA chemistry. The conjugation efficiency of this reaction was verified by reacting MTet-EVs with a fluorescent AZ488-labelled TCO probe. Single EV flow cytometry revealed that even concentrations as low as 0.001 mM AZ488-TCO were able to result in a highly efficient conjugation as evidenced by the large shift in the EV population to the right (FIG. 1(b)). Incubation of higher concentrations of AZ488-TCO up to 1 mM showed increased yield as shown by the rightward shift of the EV peak at higher concentrations. This is in agreement with reaction kinetics reported for iEDDA reactions that are shown to result in high yields even at very low substrate concentrations. The accuracy of EV detection was confirmed via the use of reagent controls and detergent controls as per MISEV 2018 guidelines (FIG. 14(d)). The iEDDA-mediated EV conjugation was further verified via the conjugation of a Biotin-TCO probe. MTet-EVs incubated with the Biotin-TCO probe showed a significant increase in fluorescence following staining with a fluorescent streptavidin conjugate, with over 99% of EVs staining positive for biotin (FIG. 1(c) to (d)). However, unmodified EVs without the MTet click handle showed no clear shift in biotin signal following incubation with Biotin-TCO. Of note, EVs were co-stained with GPA in this experiment to confirm that the biotin-positive events observed are actually contributed by RBCEVs as evidenced by the presence of a highly double-positive EV population. The Biotin-TCO-conjugated EVs were also assessed using western blot, where the relative conjugation yield at increasing concentrations of Biotin-TCO were assessed. As shown in FIG. 1(e), increasing concentrations of Biotin-TCO resulted in increasing yields of biotin per EV as evidenced by the more intense banding patterns. Quantification of the western blot revealed that each EV could be conjugated to up to 25,000 biotin molecules following iEDDA conjugation with 1 mM concentration of Biotin-TCO probes (FIG. 1f). No significant differences in the relative yield of conjugation upon reacting with EVs form different donors were observed, confirming the repeatability of the conjugation approach (FIG. 14(e)). Given the reported ultra-fast kinetics of iEDDA mediated conjugation, the time taken for the reaction to reach completion was observed. Approximately 80% of the yield was already obtained following 1 minute of incubation while the reaction had reached completion within 10 minutes as assessed using western blot (FIG. 1(g)). At the endpoint of the iEDDA reaction using 1 mM of TCO probes, over 90% yield was obtained, indicating that 90% of the MTet on the EV surface had successfully reacted with a TCO probe (FIG. 1(h), FIG. 15(f)). Assessment of stability of conjugation performed via the incubation od EVs in human plasma for a period of 48 hours revealed that incubation had little to no detrimental effects on the stability of iEDDA-mediated conjugation as there was no reduction in the biotin signal over time (FIG. 15(h)). The relative efficiency of this iEDDA-based method was compared to pre-existing post-isolation methods developed for use in the field of EV-engineering. In the past, it was required to rely on the use of enzymatic ligation via the use of protein ligases such as Sortase A heptamutant and OaEP1 Cys247Ala to create covalent functionalize the EV surface. iEDDA-mediated conjugation was shown to result in higher yields than OaAEP1-mediated conjugation with either of the peptides (FIG. 1(i)). Moreover, iEDDA was shown work better at all substrate concentrations tested to conventional Strain promoted alkyne-azide cycloaddition (SPAAC) which utilized azide and DBCO click handles (FIG. 1(j)).

The present disclosure also describes EV surface functionalisation approaches based on iEDDA (Inverse electron-demand Diels-Alder)-mediated conjugation. The conjugation method disclosed herein allows one to covalently functionalise the surface of EVs post-isolation. The iEDDA-mediated conjugation is also shown to be stable, efficient, translatable to any protein of interest, and resulted in increased copy numbers over existing methods of bioconjugation for surface functionalisation developed thus far. Further functional studies described in the present disclosure have shown that ligands conjugated on the EV surface maintained their functionality. Conjugation of ligands on the EV surface led to multimerisation of these ligands, resulting in an increase in receptor crosslinking upon EV binding and endocytosis. Multimerisation of these ligands on the EV surface results in the clustering of a large number of these ligands in close proximity on the EV surface. Subsequently, when these ligands bind to their target receptor on a cell, the presence of the large number of ligands in close proximity is thought to induce receptor crosslinking, which in turn increases intracellular signalling compared to an equivalent amount of free soluble ligand.

In one example, the method disclosed herein functionalises the surface of the EVs using STP esters. In another example, the present invention disclosed an EV surface functionalisation approach based on iEDDA (Inverse electron-demand Diels-Alder)-mediated conjugation that is able to covalently functionalise the surface of EVs post-isolation. One advantage of the EV surface functionalisation approach (method) as disclosed herein is that the EVs do not require the presence of a scaffold protein in order to attach the immunomodulatory molecules to the surface of the EVs. Another advantage of the approach disclosed herein is that the immune cells can be stimulated by the immunomodulatory molecules conjugated to the surface of the EVs. Thus, no intracellular delivery of agents to the immune cells is required.

Also disclosed herein are methods of obtaining the claimed nanoscale extracellular. In one example, the method comprising the steps of: a. isolating nanoscale extracellular vesicles from a cell or a compound capable of forming extracellular vesicles; b. attaching a click chemistry handle to the nanoscale extracellular vesicle surface; c. attaching a corresponding click chemistry handle to the immunomodulatory molecule; and d. conjugating the click chemistry handle of step b to the corresponding click chemistry handle of step c; thereby anchoring the immunomodulatory molecules to the surface of the nanoscale extracellular vesicle. In another example, the method further comprises the steps of: i. permeabilising the nanoscale extracellular vesicle of step a. or step d. with dimethyl sulfoxide (DMSO); and ii. incubating the nanoscale extracellular vesicle obtained from step i. with a payload.

In one example, the nanoscale extracellular vesicle can be permeabilised with dimethyl sulfoxide (DMSO). In another example, the order of these steps (permeabilising the EV, incubating with a payload, and attaching the click chemistry handle or immunomodulatory molecule) is flexible. In one example, the order is to first permeabilise the EV, then load it with the payload, and finally attach the click chemistry handle or immunomodulatory molecule. Alternatively, the click chemistry handle or immunomodulatory molecule can be attached to the EV first, followed by permeabilising it and loading it with the payload.

In one example, the nanoscale extracellular vesicle as disclosed herein comprises click chemistry handles. Examples of click chemistry handles can be, but are not limited to, dibenzocyclooctynes (DBCO), transcyclooctene (TCO), methyltetrazine (MTet), tetrazines, azide, alkynes, 9,10-phenanthrenequinones (PQs), electron-rich vinyl ethers (VEs), thiols, and maleimides. In one example, the click chemistry handle is methyltetrazine (MTet) or transcyclooctene (TCO). In another example, the click chemistry handle is methyltetrazine (MTet), transcyclooctene (TCO), or a variant thereof, such as tetrazine. The variant can include, but is not limited to, tetrazine, which is the non-stabilised form of methyltetrazine.

In one example, the nanoscale extracellular vesicle as disclosed herein comprises one or more immunomodulatory molecules anchored to the nanoscale extracellular vesicle using a corresponding click chemistry handle. Examples of the corresponding click chemistry handles can be, but are not limited to, dibenzocyclooctynes (DBCO), transcyclooctene (TCO), methyltetrazine (MTet), tetrazines, azide, alkynes, 9,10-phenanthrenequinones (PQs), electron-rich vinyl ethers (VEs), thiols, and maleimides. In one example, the click chemistry handle and the corresponding click chemistry handle are different from each other. In another example, the corresponding click handle is a transcyclooctene (TCO) click chemistry handle.

### EDDA-Functionalised EVs Display Proteins at High Copy Number on the EV Surface

Also disclosed herein is the optimisation of iEDDA-mediated EV functionalisation and the measurement of protein conjugation efficiency to EVs using TCO probes. Optimisation of the conjugation process was achieved by comparing the conjugation efficiency after varying a range of parameters known to affect these bioconjugation reactions, including pH, substrate concentration, EV concentration, temperature, and reaction time.

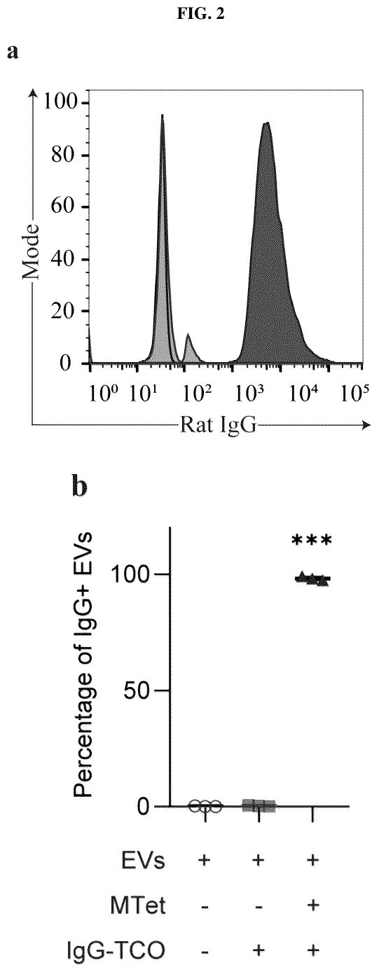

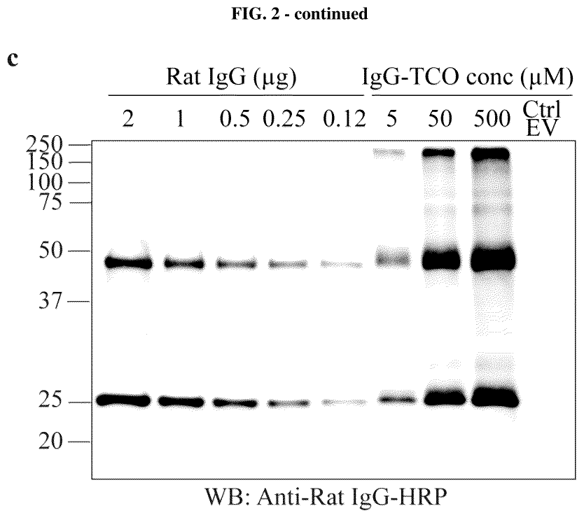

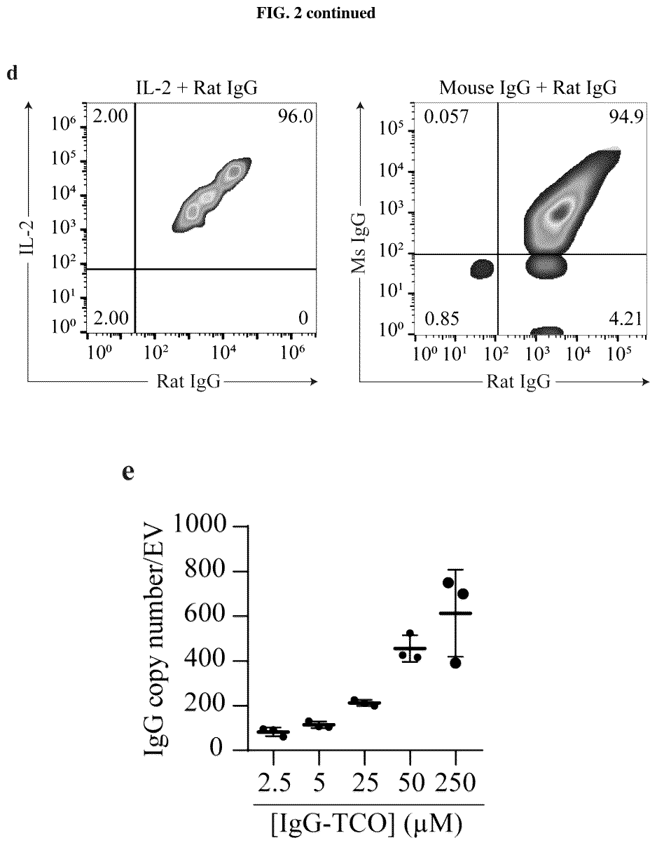

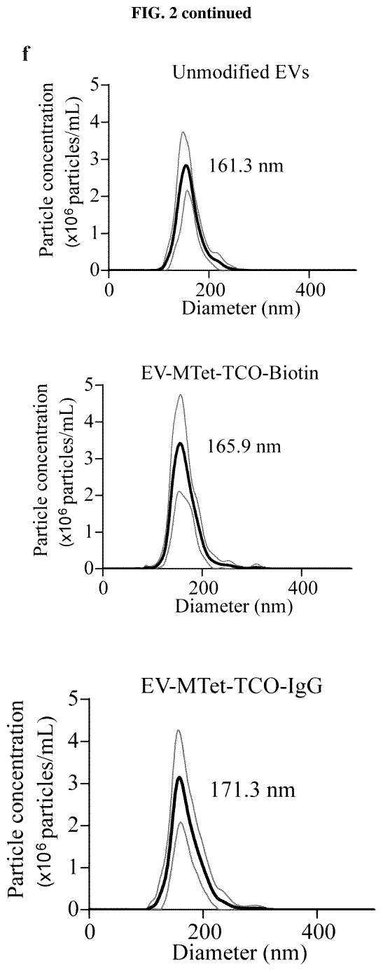

After optimising iEDDA-mediated EV functionalisation and confirming its efficacy using TCO probes, the translatability of this approach was verified for the conjugation of TCO-labelled proteins. In one example, a TCO-labelled rat IgG was conjugated onto MTet EVs and the conjugation efficiency was verified using single EV flow cytometry. Staining with an anti-rat secondary antibody revealed a clear shift of the EV population to the right indicating that ~98% of the EVs were successfully conjugated with the antibody (FIG. 2(a) to (b)). It was also confirmed the covalent nature of the conjugation using western blotting. Western blotting performed under reducing conditions revealed that ~25% of the heavy and light chains had increased in molecular weight to form a smeared banding pattern, while the remaining 75% of the heavy and light chains only showed a slight increase in molecular weight as evidenced by the minor shift of the band (FIG. 2(c)). The reason for this pattern of banding is because one of the four IgG chains would be covalently conjugated at random to an EV protein via iEDDA-mediated conjugation resulting in a smearing pattern while the remaining IgG chains would only display minor increases in molecular weight due to the incorporation of TCO click handles. Thus, the Western blot confirms the covalent nature of the conjugation. To further verify that this conjugation approach could be used to conjugate multiple ligands on the EV surface, equimolar quantities of TCO-labelled rat IgG and IL-2 or rat IgG and mouse IgG were mixed. The percentage of double positive EVs was subsequently determined using single EV flow cytometry. In both cases, a double positive population of EVs of 95-96% was shown to be present, thus confirming that multiple ligands could be conjugated on a single EV with high efficiency (FIG. 2(d)). The copy number of IgG molecules conjugated per EV was also determined following incubation with varying concentrations of TCO-IgG in the iEDDA reaction. It was observed that even the lowest IgG concentration of 2.5 μM used resulted in over 80 copies of IgG per EV (FIG. 2(e)). Increasing IgG concentrations resulted in increasing yields with the highest concentration of 250 UM resulting in ~600 copies of IgG per EV. Nanoparticle tracking analysis was performed to determine if the conjugation significantly affected EV size or induced EV aggregation. The data shows that ester reactions with Methyltetrazine-STP increased the average diameter of EVs by about 4.6 nm, which was further increased by 5.4 nm upon antibody conjugation FIG. 2(f). The induction of EV aggregation was not observed as the EV size distribution profile appeared to be similar before and after conjugation.

Using a model of CD3 and CD137 agonistic antibodies, the present disclosure demonstrated that the conjugation of immunomodulatory ligands onto the EV surface enhanced the ligands efficacy as compared to equivalent doses of free ligand. Additionally, the present disclosure demonstrates that conjugation of multiple complementary ligands on the same EV surface resulted in enhanced crosstalk via the formation of immune synapses between the ligand-functionalised EV membrane and the T-cell membrane receptors, thereby allowing enhanced immune activation.

In one example, when compared to known EV surface engineering methods, the iEDDA-mediated conjugation disclosed herein increases copy numbers of proteins attached onto the EV surface. Moreover, the iEDDA-mediated conjugation disclosed herein is safe and biocompatible showing no indications of immunogenicity. The conjugation between EVs and the protein of interest is stable and covalent. Additionally, the iEDDA conjugation disclosed herein exhibits high first order reaction kinetics, enabling reactions to occur in as little as 5 minutes, even with low concentrations of TCO-labelled substrate.

In another example, when compared to free ligands, surface display of immunomodulators on EVs improves systemic biodistribution and reduces the onset of off-target toxicity.

### Display of Immunomodulatory Ligands on EV Surface Enhances Efficacy

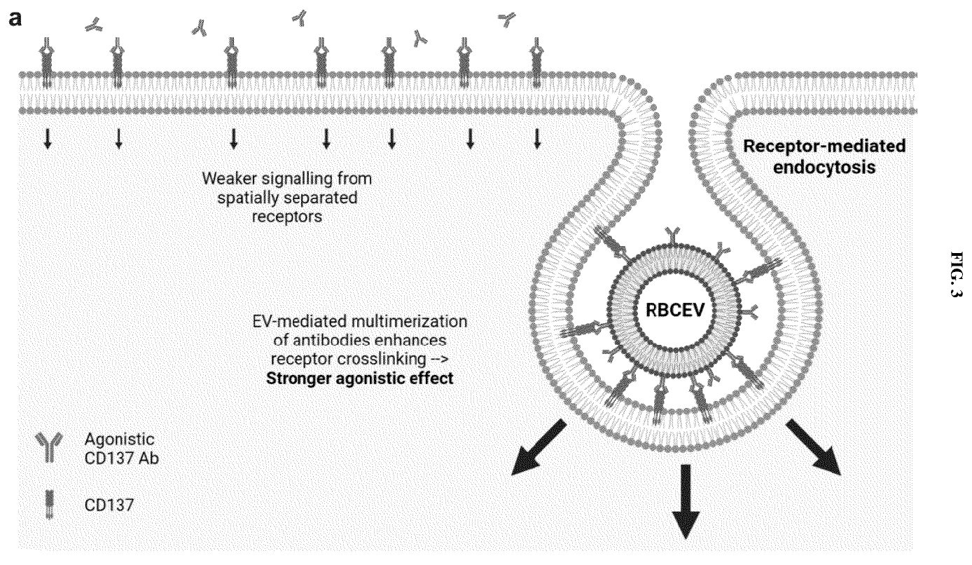

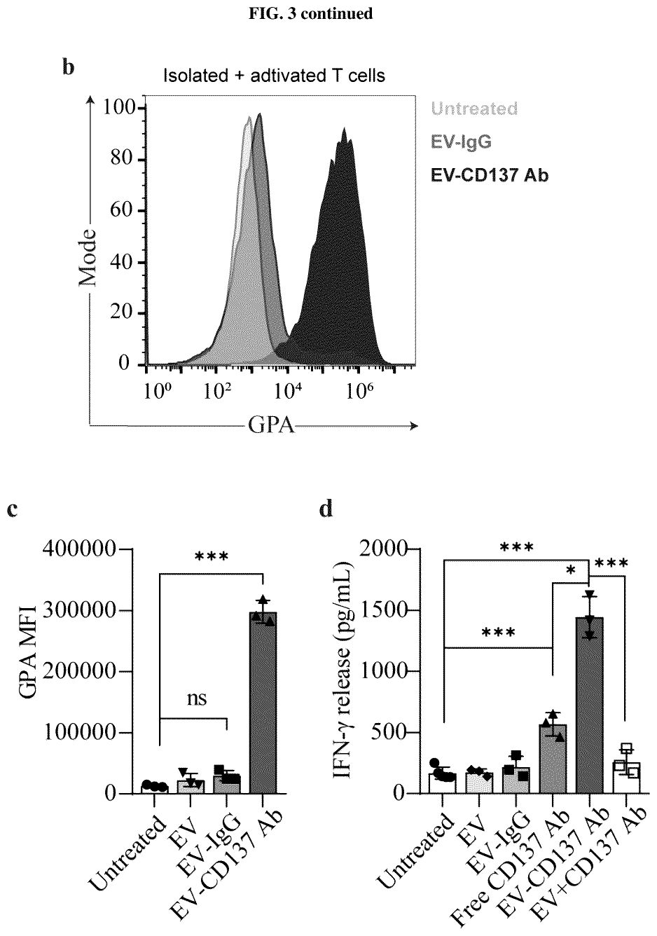

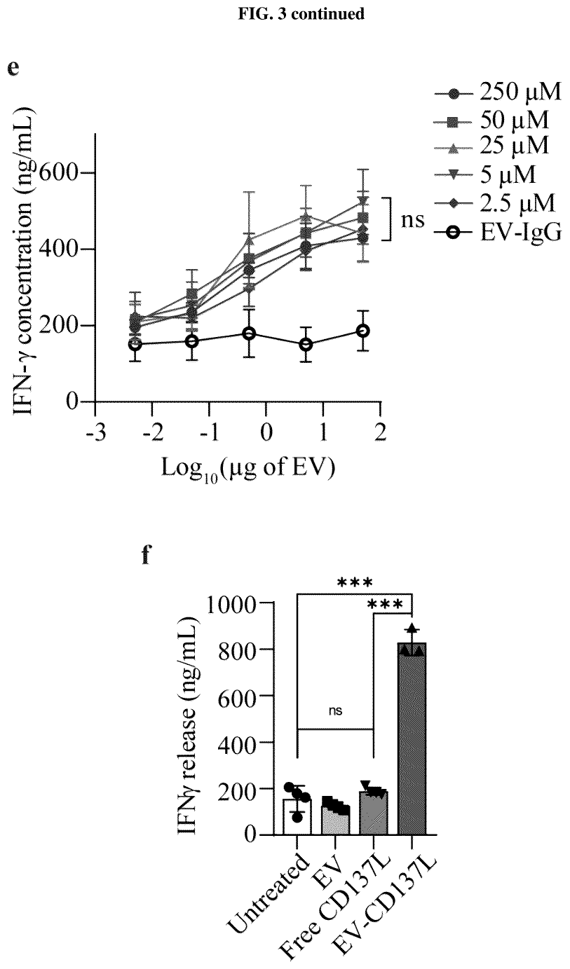

It was hypothesised that conjugating certain cytokines or agonistic antibodies onto the EV surface would enhance their therapeutic efficacy by enhancing receptor crosslinking mediated by the antibody multimerisation effect created by their surface display on the EV surface (FIG. 3(a)). To verify this, an agonistic CD137 antibody that was capable of binding to CD137 on activated T-cells and inducing IFN-γ release was used. The functionality of the agonistic CD137 antibody after conjugation on the EV surface was first verified by verifying its ability to bind to activated T-cells. CD137-conjugated EVs (EV-CD137 Ab), but not isotype antibody-conjugated EVs (EV-Iso Ab) were able to significantly increase the binding of EVs to activated T-cells expressing CD137, confirming that antibodies conjugated on the EV surface maintained their binding affinity to their target antigens (FIG. 3(b) to (c)). Next, the relative efficacy of EV-CD137 or an equivalent quantity of free CD137 agonistic antibody at inducing IFN-γ release from T-cells following 24 hours of stimulation was determined. The EV-CD137 treatment resulted in a ~2.5-fold increase in IFN-γ release as compared to free antibody treatment, confirming that simply conjugating certain immunomodulatory ligands on the EV surface can enhance their therapeutic efficacy (FIG. 3(d)). Of note, isotype control antibody conjugated EVs (EV-IgG) or EVs simply incubated with CD137 and washed had no significant effect on IFN-γ release. Dose response analysis of agonistic CD137 antibody-conjugated EVs revealed a sigmoidal dose response, with EV concentrations as low as 50 ng/ml resulting in detectable levels of IFN-gamma release (FIG. 3(e)). However, an effect of increasing antibody copy number per EV was not observed, as there was no difference in IFN-gamma released by EVs conjugated at varying concentrations of TCO-labelled CD137 agonistic antibody at any of the tested doses. This indicates that the copy number obtained at the lowest concentration (about 80 antibodies per EV) was sufficient to obtain maximal CD137 signalling and further increases in copy number had no additional effect. It was also intended to determine if EV conjugation could enhance the immune stimulatory effect of other classes of immunotherapeutics such as ligands and cytokines. Conjugation of soluble CD137L on EVs displayed similar enhancements as compared to equivalent quantities of free soluble CD137L, indicating that EV conjugation-mediated increase of stimulatory potential was not exclusive to agonistic antibodies (FIG. 3(f)). Similar increase in stimulatory potential were also observed in the case of mouse IL-2, as demonstrated by the increase in T-cell proliferation observed in EV-IL-2 treatments as compared to free IL-2.

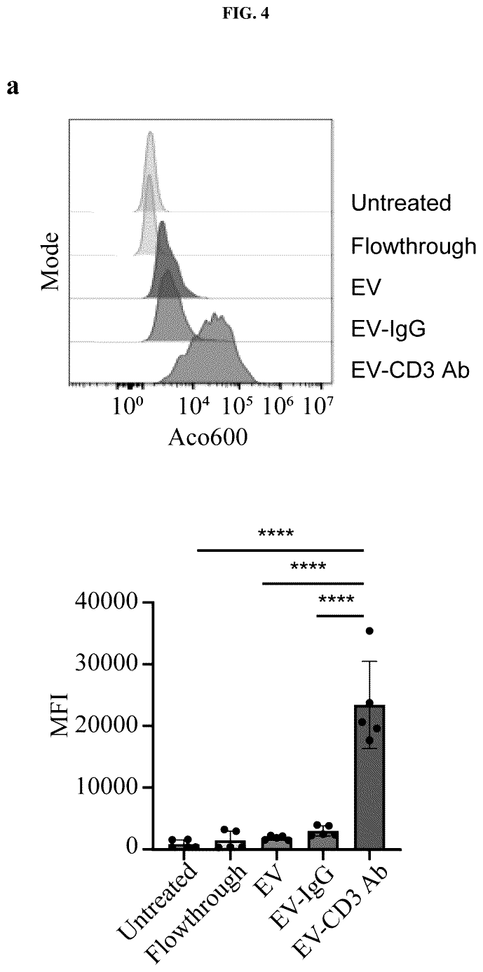

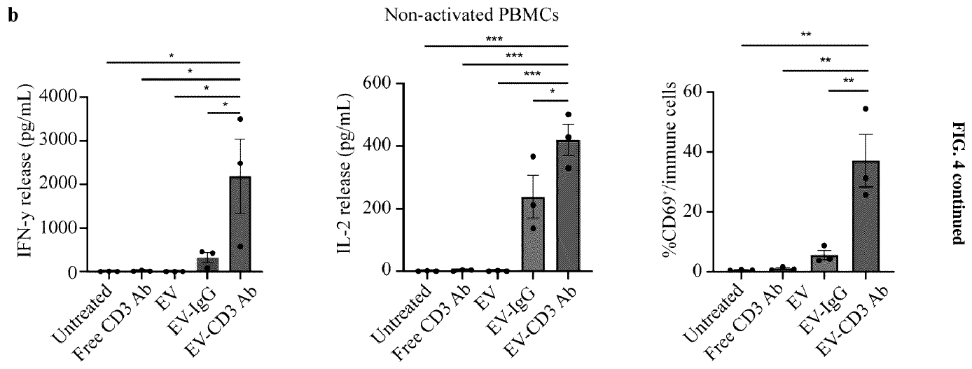

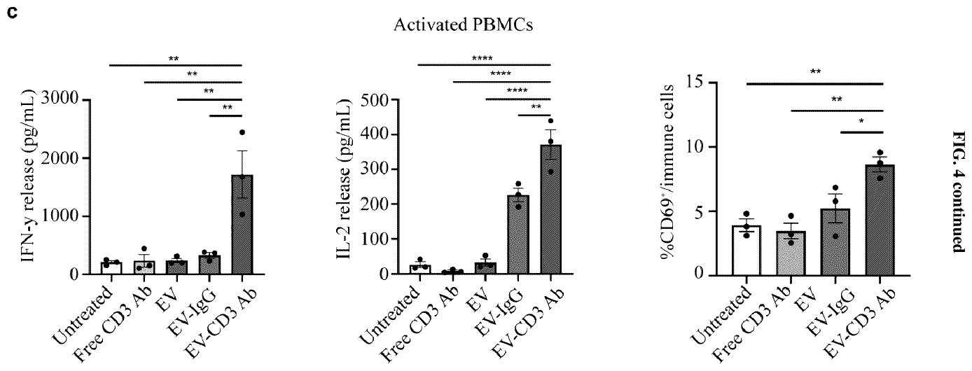

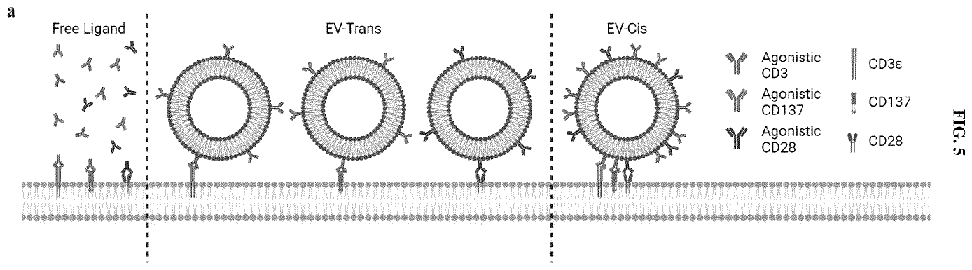

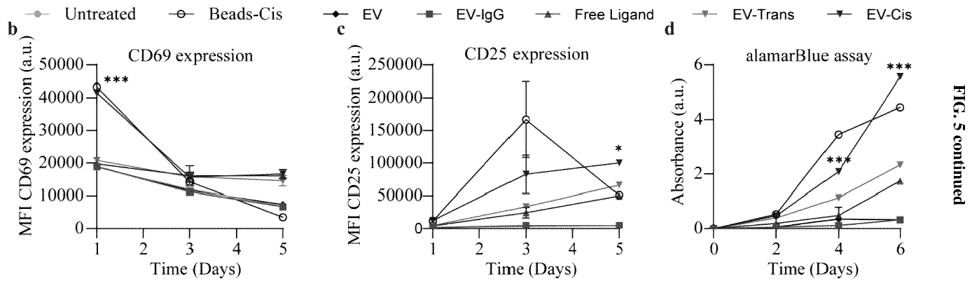

The immunomodulatory function of the system in human PBMCs was further investigated using human αCD3-conjugated EVs (EV-CD3 Ab). Of note, EVs were labelled with Acoerela fluorescent dye (Aco-600) and washed before incubation with PBMCs. Treatment with EV-CD3 Ab significantly enhanced targeting efficiency to CD3⁺ T cells in PBMCs (FIG. 4(a)). In both non-activated and activated PBMCs, treatment with EV-CD3 Ab resulted in increases in IFN-γ and IL-2 release as compared to free antibody treatment at the same dosage (FIG. 4(b) to (c)). Additionally, upregulation of CD69, an early activation marker, was observed in samples treated with EV-CD3 Ab (FIG. 4(b) to (c)).

In one example, conjugation of immunomodulatory ligands on the EVs surface mediated multimerisation of ligands and facilitates receptor crosslinking for more efficient signalling. In another example, multiple immunomodulatory ligands can be conjugated on the EV surface, resulting in an increase in anti-tumour responses. In yet another example, loading immunomodulatory ligands onto the EV surface limits the biodistribution of immunomodulators to the local tumour microenvironment, thus limiting systemic toxicity. This allows for lower doses and more potent ligand combinations to be used for improved efficacy while maintaining an enhanced safety profile.

### Conjugation of Multiple Complementary Ligands in Cis on the EV Surface Enhances Signalling Efficiency

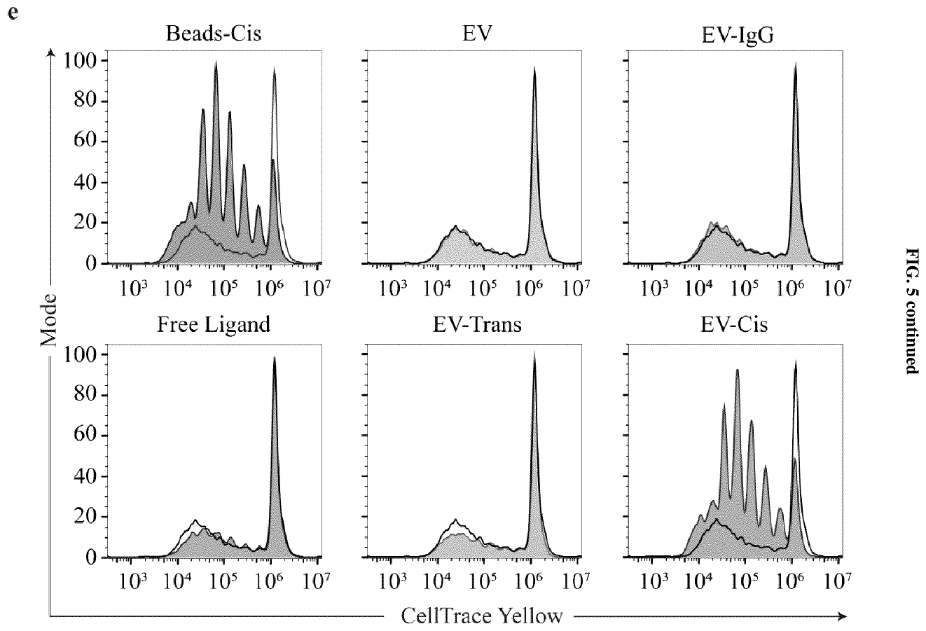

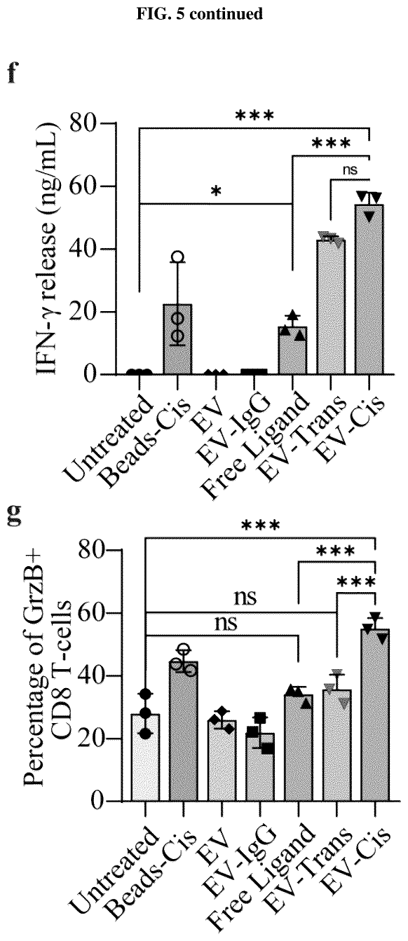

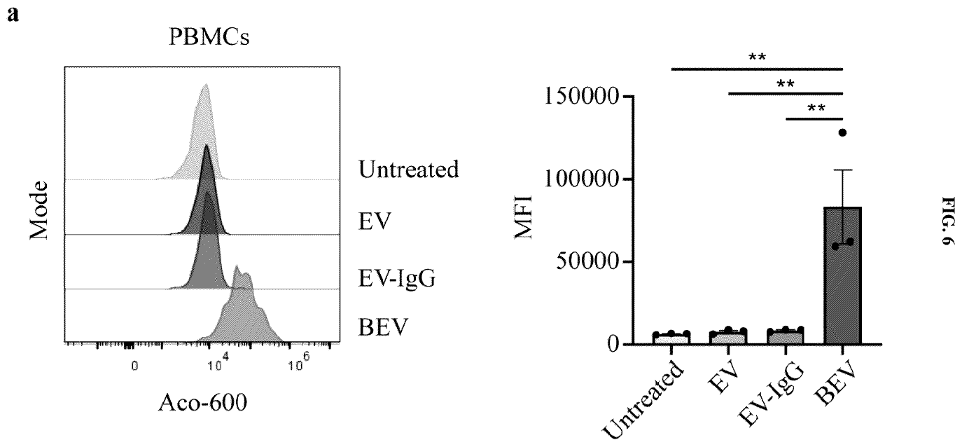

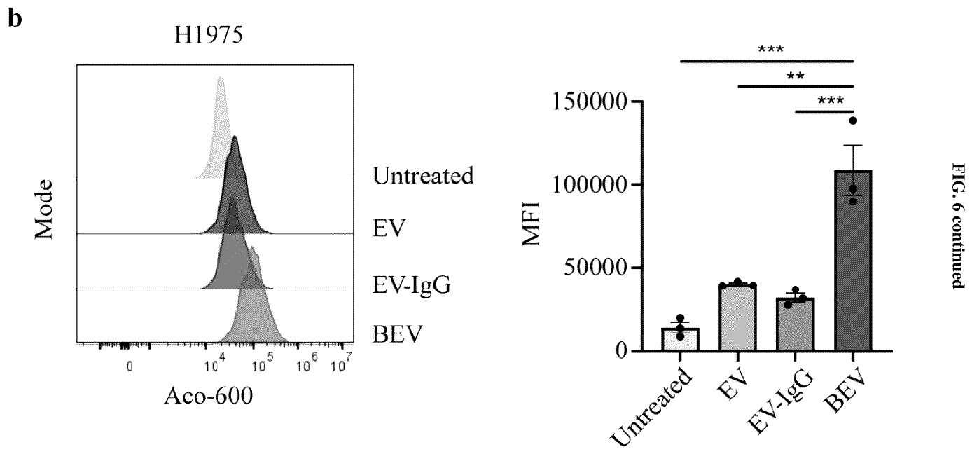

To determine the optimal orientation for delivering multiple complementary ligands to a specific target cell, an experiment was designed to compare the relative efficacy of cis vs trans display for three agonistic antibodies that work synergistically for achieve T-cell activation—CD3, CD137, and CD28 (FIG. 5(a)). A series of assays were performed to determine whether EV-Cis were superior to EV-Trans and how both of them would compare to an equivalent quantity of free ligands. It was observed that CD69 expression (a very early T-cell activation marker was upregulated highly on day 1 following treatment with EV-Cis to a level comparable to the positive control of Beads-Cis (FIG. 5(b)). A similar trend was observed with CD25 where the expression of CD25 peaked on day 5 following treatment with EV-Cis. Interestingly, the positive control of Beads-Cis peaked at Day 3 before dropping (FIG. 5(c)). EV-Trans and Free Ligand treatments showed significantly lower levels of upregulation of activation markers while the negative controls of EV only and EV-IgG did not show any upregulation. T-cell proliferation monitored via alamarBlue assay revealed a similar trend with Cis-EVs resulting in significantly higher levels of T-cell proliferation than any of the other treatment conditions on day 6 (FIG. 5(d)). Beads-Cis also showed potent T-cell proliferation, though the increase in T-cell proliferation decreased between day 4 and day 6. EV-Trans and Free Ligand treatments also induced T-cell proliferation though significant proliferation was only observed after Day 4, with EV-Trans demonstrating higher levels of proliferation than Free Ligand treatment. This trend was reflected in the CellTrace dilution assay where generations a far as 6 were discerned in EV-Cis and Beads-Cis treatments as early as Day 3 while EV-Trans and Free Ligand treatments did not show clear generational dilution of CellTrace dye (FIG. 5(e)). This trend continued in the levels of IFN-γ release in the supernatant and Granzyme B expression (FIG. 5(f) to (g)). Interestingly, while EV-Cis was still the more potent treatment, EV-Trans treatment induced comparable levels of IFN-γ release which was significantly higher that Free Ligand treatment, a trend that was not observed in Granzyme B expression. Taken together this data confirms that Cis-display on EVs is the most efficient way to deliver ligands for achieving maximal T-cell stimulatory effect.

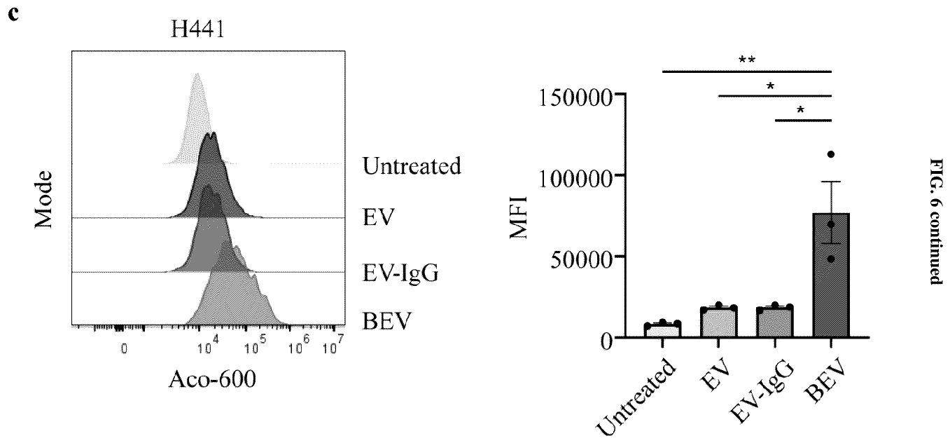

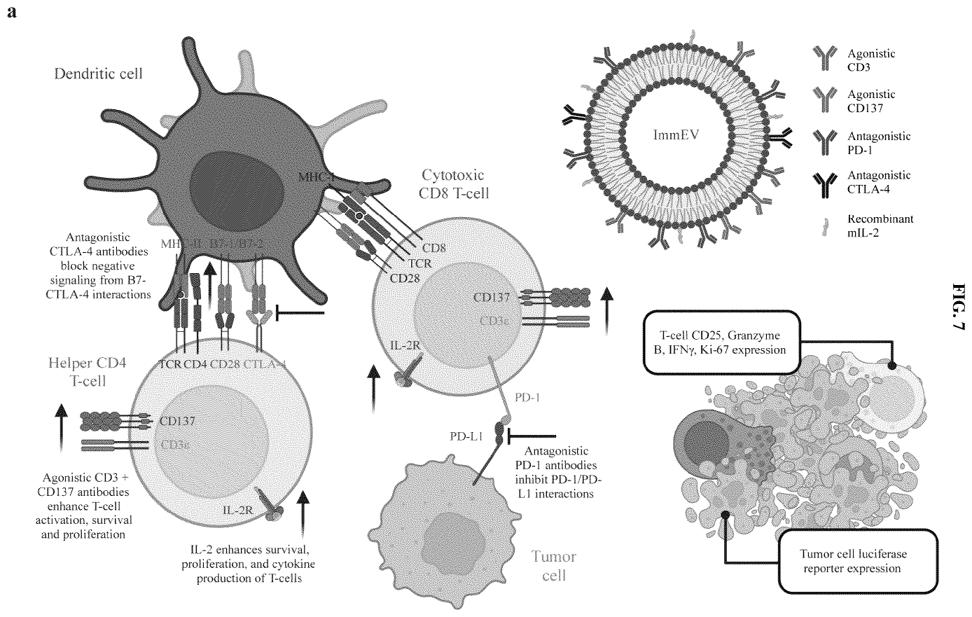

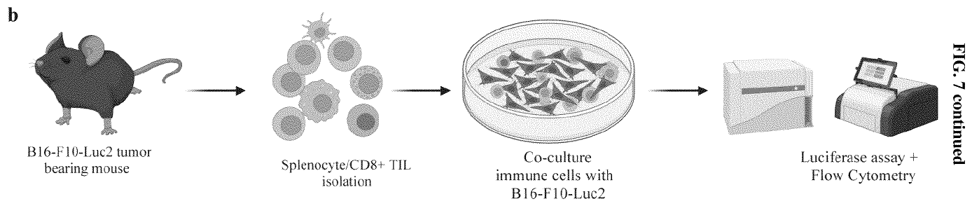

To extend the applicability of the method and platform disclosed herein, αCD3 and αPD-L1 antibodies were conjugated in cis display on EV's surface to generate bispecific EVs (BEVs) to target both T cells and tumour cells expressing PD-L1, an immune checkpoint molecule upregulated in many cancers. As shown by FIG. 6(a), bispecific EV-Cis (BEVs) could target T cells in human PBMCs with higher efficiency than control groups. Treatment with EV-Cis also resulted in increased targeting efficiency in two human lung cancer cell lines including H1975 and H441 which highly express PD-L1 (FIG. 6(b) to (c)). This design aims to facilitate the recruitment of activated T cells to tumour cells, which may help to transform “cold” tumour into “hot” tumour.

In one example, the nanoscale extracellular vesicle disclosed herein comprises two types of immunomodulatory molecules anchored to the nanoscale extracellular vesicle; and wherein the first type of immunomodulatory molecule is an agonistic antibody, and the second type of immunomodulatory molecule is an antagonistic antibody. In another example, the agonistic antibody is anti-CD3 antibody, and the antagonistic antibody is anti-PD-L1 antibody. In yet another example, the anti-CD3 antibody and the anti-PD-L1 antibody comprise a transcyclooctene (TCO) click handle. In one example, the agonistic antibody is anti-CD137 antibody, and the antagonistic antibody is anti-PD-L1 antibody. In another example, the anti-CD137 antibody and the anti-PD-L1 antibody comprise a transcyclooctene (TCO) click handle.

In one example, the nanoscale extracellular vesicle is as disclosed herein, wherein three types of immunomodulatory molecules are anchored to the nanoscale extracellular vesicle; wherein the first type of immunomodulatory molecule is an agonistic antibody, the second type of immunomodulatory molecule is an antagonistic antibody, and the third type of immunomodulatory molecule is a cytokine. In another example, the agonistic antibody is CD3 and CD137 antibodies; the antagonistic antibody is PD1 and CTLA-4 antibodies; and the cytokine is IL-2. In yet another example, the two or more immunomodulatory molecules are bound to the nanoscale extracellular vesicle surface in cis-conformation.

EV-Bound Ligands Induce More Potent Anti-Tumour Responses than Free Ligands

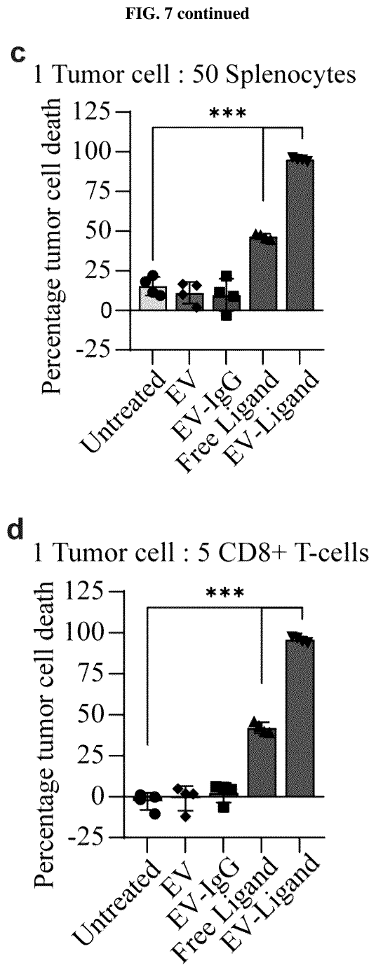

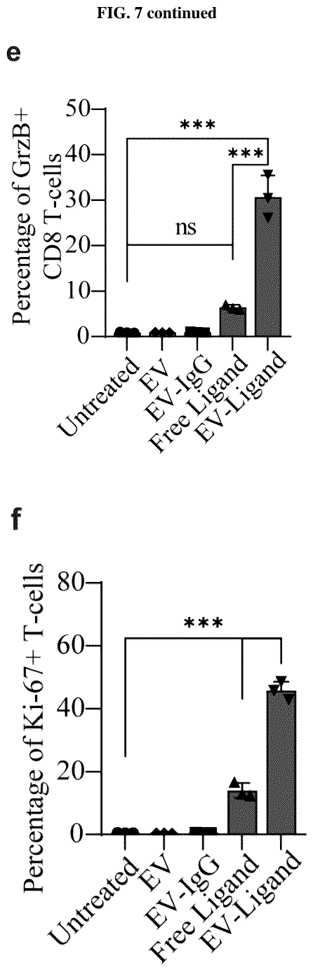

To verify the efficacy of EVs in delivering immunomodulators for anti-tumour effect a B16-F10 mouse melanoma model, which is a common, highly metastatic immunologically cold tumour model, was utilised. Immunophenotyping of lung metastases formed by B16-F10 revealed that the tumour cells expressed very high levels of PD-L1, had a high proportion of Tregs and poor T cell activation and cytotoxicity. A combination of immunomodulatory ligands was thus developed that were designed to address these inhibitory factors and enhance T-cell-mediated anti-tumour responses. This combination included agonistic CD3 and CD137 antibodies, recombinant mouse IL-2 and antagonistic PD-1 and CTLA-4 antibodies (FIG. 7(a)). These EVs termed ImmEVs or EV-Ligand were incubated with splenocytes or CD8-positive tumour infiltrating lymphocytes from B16-F10-Luc2 tumour bearing mice and co-cultured with B16-F10-Luc2 tumour cells (FIG. 7(b)). Tumour cell death was monitored using a luciferase assay. The EV-Ligand treatment was observed to be able to induce higher levels of tumour cell death as compared to treatment with an equivalent quantity of the free ligand (FIG. 7(c) to (d)). Control treatments of EVs of EV-IgG showed no significant difference form untreated co-cultures and had minimal effects on tumour cell cytotoxicity. The comparable cytotoxicity obtained in the co-culture of CD8 T-cells indicates that much of the cytotoxic effects observed in the splenocyte co-culture were mediated via the actions of stimulated CD8-positive cytotoxic T-cells. Upregulation of Granzyme B (a key marker of T-cell cytotoxicity) in CD8 T-cells and Ki-67 (a marker of proliferation) in total T-cells was observed following treatment with EV-Ligand (FIG. 7(e) to (f)). Free ligand treatments had a significantly lower effect while the control treatments were unable to induce expression of these genes. Thus, it was confirmed that the EV-Ligand preparation termed ImmEVs developed herein are able to induce immune activation and anti-tumour effects ex vivo.

Bispecific EV-Cis Conjugated with αCD3 and αPD-L1 Antibodies (BEVs) Activate and Redirect T Cells to the Tumour Sites

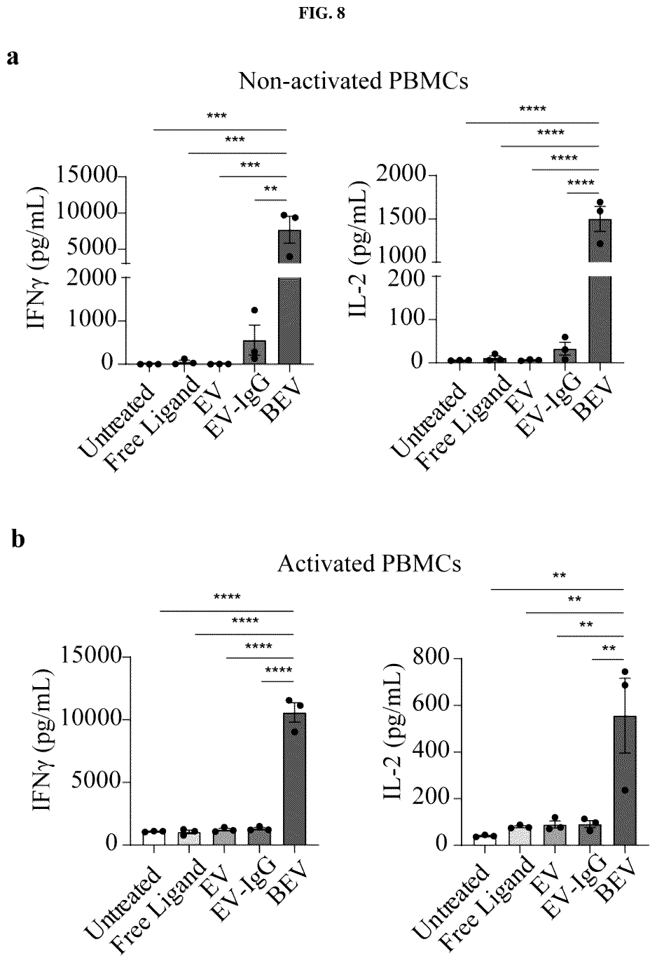

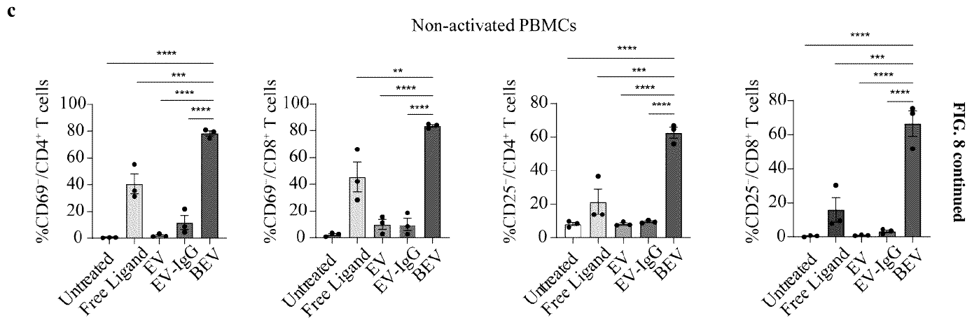

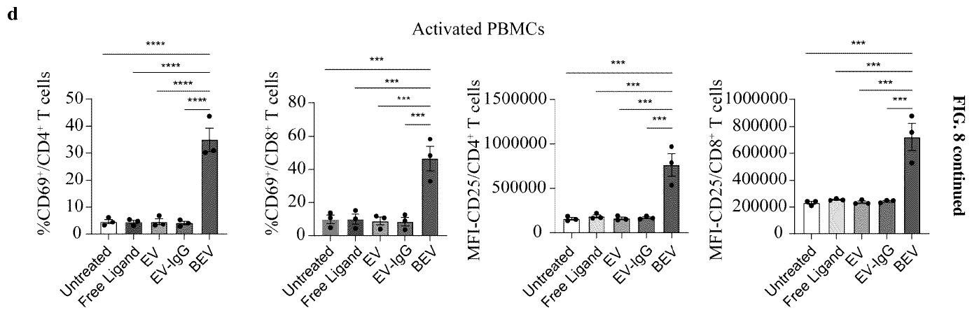

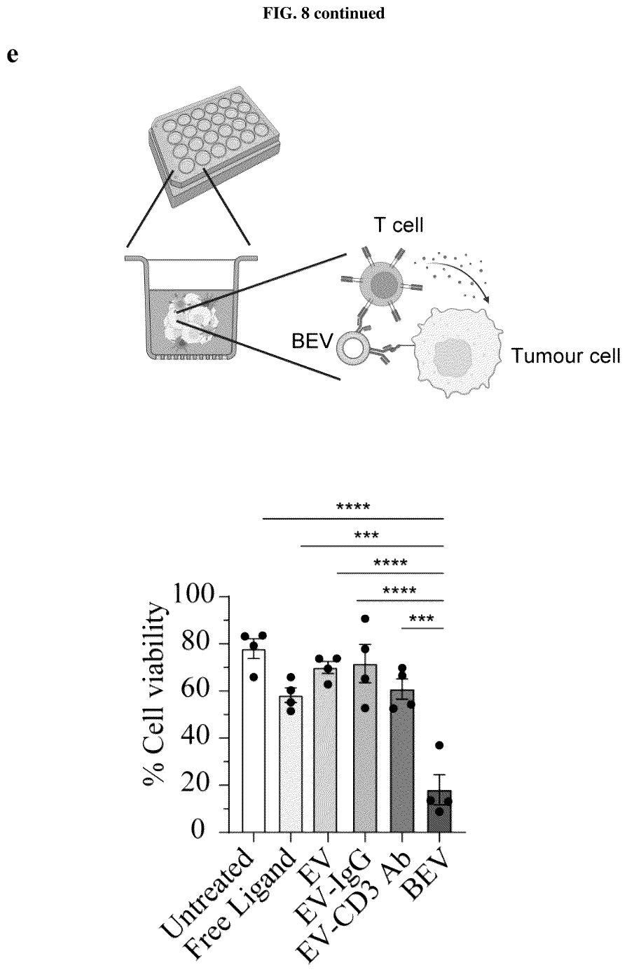

Similar to ImmEVs, potent stimulatory effects of BEVs were observed on both human non-activated and activated PBMCs as evidenced by the increase in the level of IFN-γ and IL-2 (FIG. 8(a) to (b)). T-cell activation was further quantified by measuring the expression of CD69 and CD25 on T cells as a marker for early and late T-cell activation, respectively. In non-activated PBMCs, BEVs induced a significant increase in CD69 and CD25 expression on CD4⁺ or CD8⁺ T cells compared with the control groups (FIG. 8(c)). Although free ligands could stimulate T cells to a certain extent, only BEVs could further boost T-cell activation in activated samples (FIG. 8(d)), suggesting the capability of BEVs to reinvigorate exhausted T cells in the tumour microenvironment.

BEVs were further examined for their ability to suppress tumour growth from H1975 target cells in co-cultures with PBMCs. Bioluminescent assay revealed a significant reduction in tumour viability in the BEV treatment group compared to other control conditions (FIG. 8(e)). BEVs showed a superior anti-tumour efficacy to free ligands and EV-CD3 Ab, which can be attributed to both targeting moieties on BEVs including CD3 agonistic Ab that activates T cells and anti-PD-L1 Ab that recruits T cells to the tumour cells, as well as blocking PD-L1 and PD-1 interaction to prevent exhaustion of T cells.

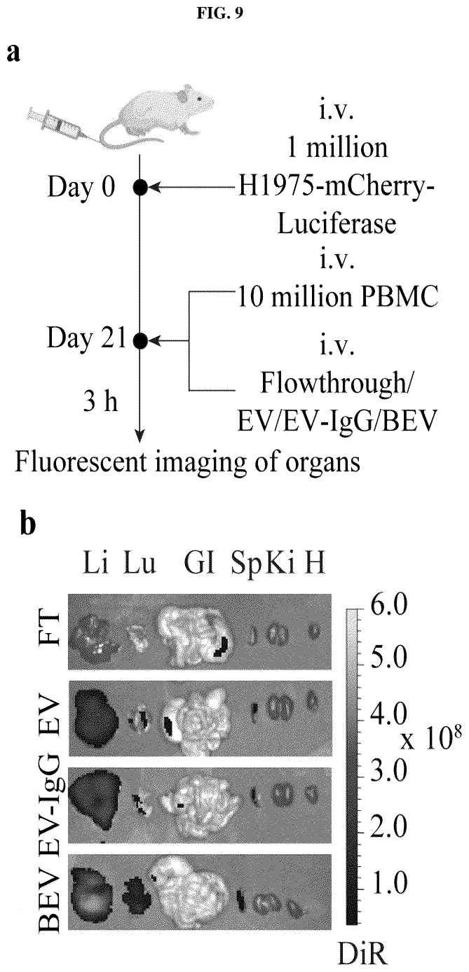

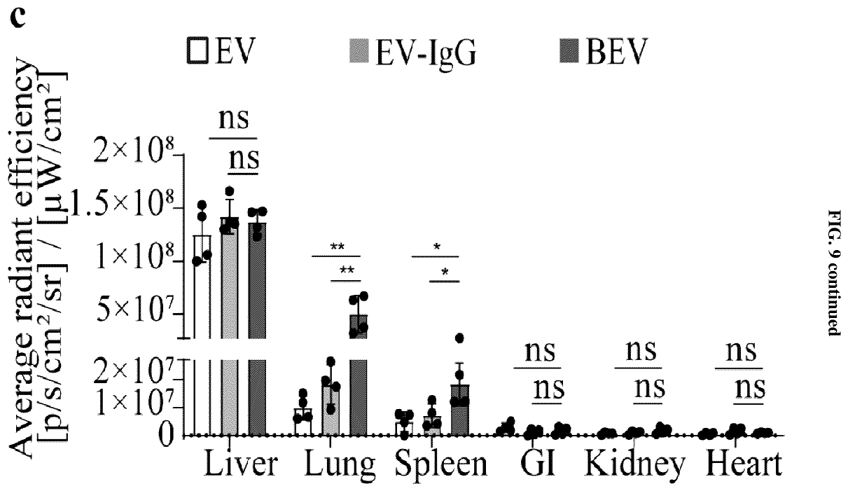

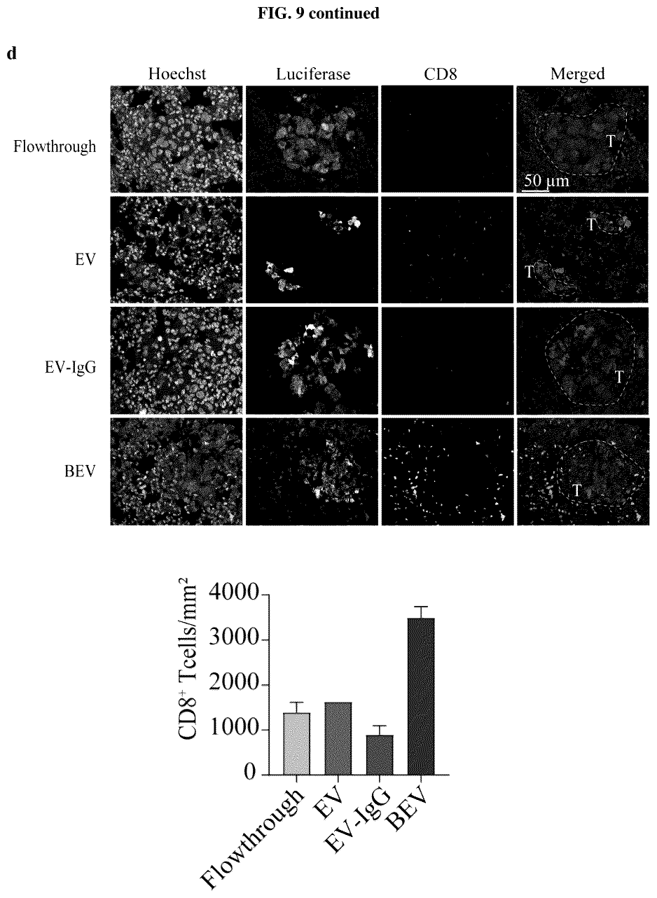

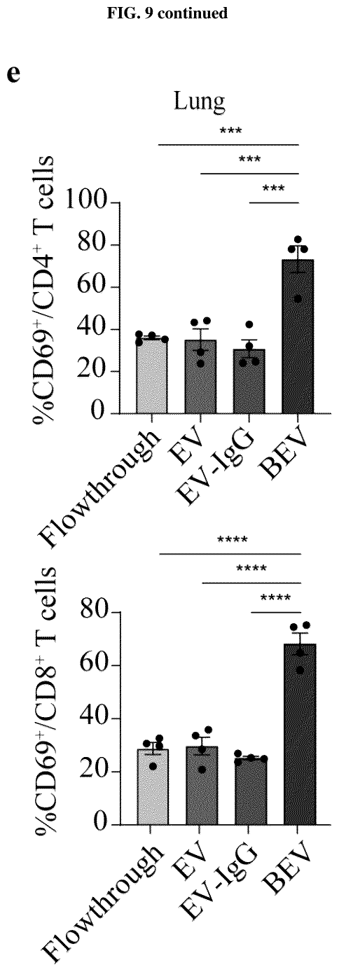

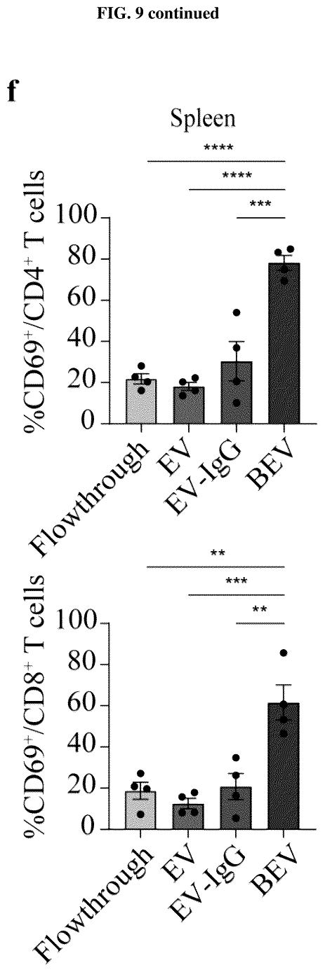

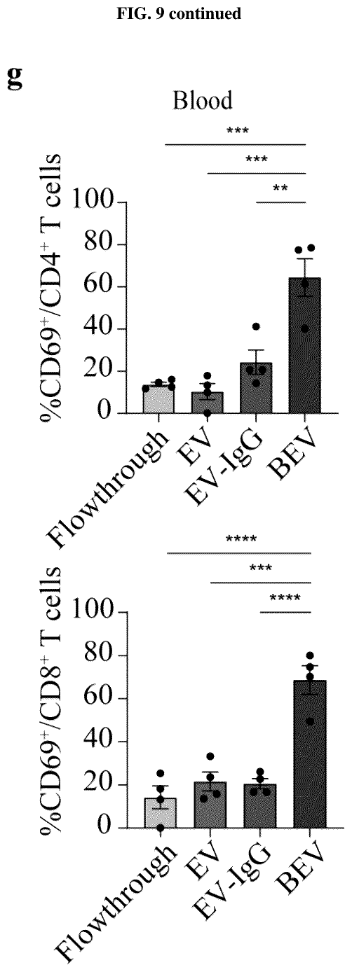

Having demonstrated the functionality of BEV in vitro, in vivo characterisation of BEV NSG-SGM3 mice bearing human lung cancer cells H1975 expressing mCherry and luciferase (H1975-mCherry-Luc) was performed. After successful engraftment of H1975-mCherry-Luc cells, human PBMCs were intravenously injected, followed by a single dose of DiR-labelled EVs, IgG-conjugated EV, or BEVs (FIG. 9(a)). The flowthrough, which was the supernatant from the last wash of DiR-labelled EVs, was used as a background control. After 3 hours, we measured DiR fluorescence in the harvested organs (FIG. 9(b)). Overall, EVs from all groups mainly accumulated in the liver, which is consistent with previous studies. Mice treated with BEVs had an increased fluorescence radiance signal in the lung and spleen compared with other control groups (FIG. 9(c)), suggesting that BEVs readily distribute in living animals and localize at the target sites. Fluorescent microscopy analysis further showed higher infiltration levels of CD8⁺ T cells at tumour regions of mice receiving the BEV treatment (FIG. 9(d)). Additionally, only BEVs activated T cells as shown by the upregulation of CD69 on both CD4⁺ and CD8⁺ T cells from the lung (FIG. 9(e)), spleen (FIG. 9(f)), and blood (FIG. 9(g)) while other treatments elicited low background activation. Taken together, these results indicate that BEVs bind and activate T cells and recruit them to tumour sites in vivo.

In one example, the bispecific EVs conjugated with anti-CD3 and anti-PD1 antibodies as disclosed herein can be administered to a subject via intravenous (IV) injection. In another example, the bispecific EVs conjugated with anti-CD3 and anti-PD1 antibodies as disclosed herein can be administered to a subject via intratracheal administration.

Local Administration of Ligand-Conjugated EVs Limits their Exposure to the Immediate Tumor Microenvironment and Abrogates Systemic Toxicity

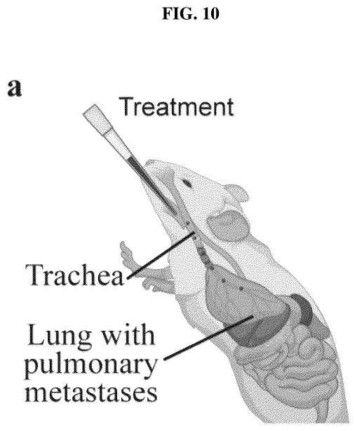

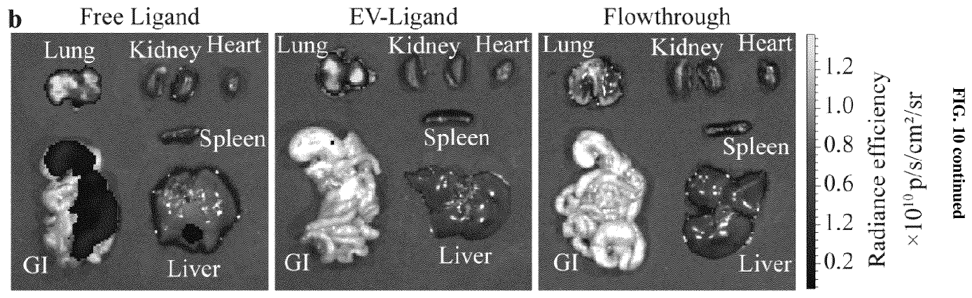

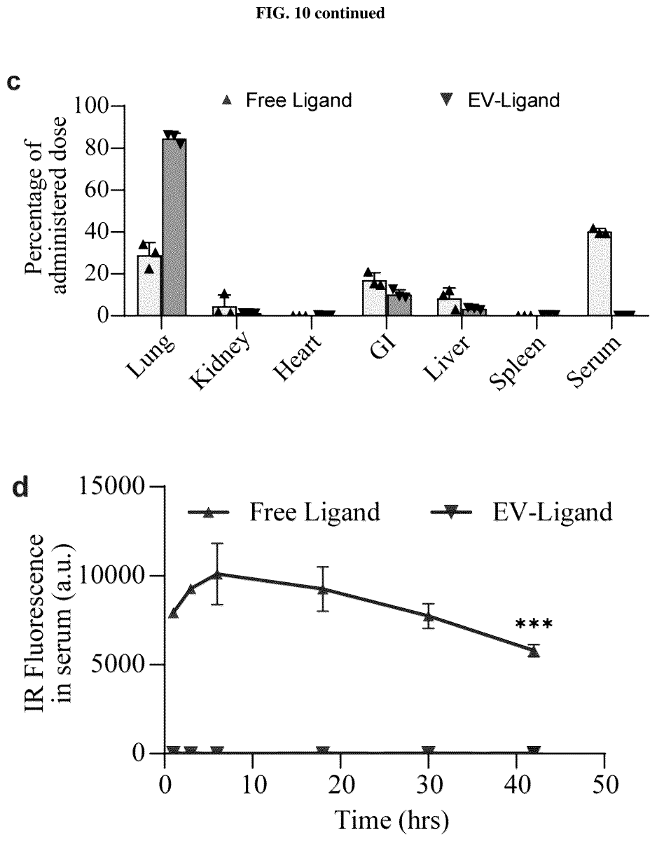

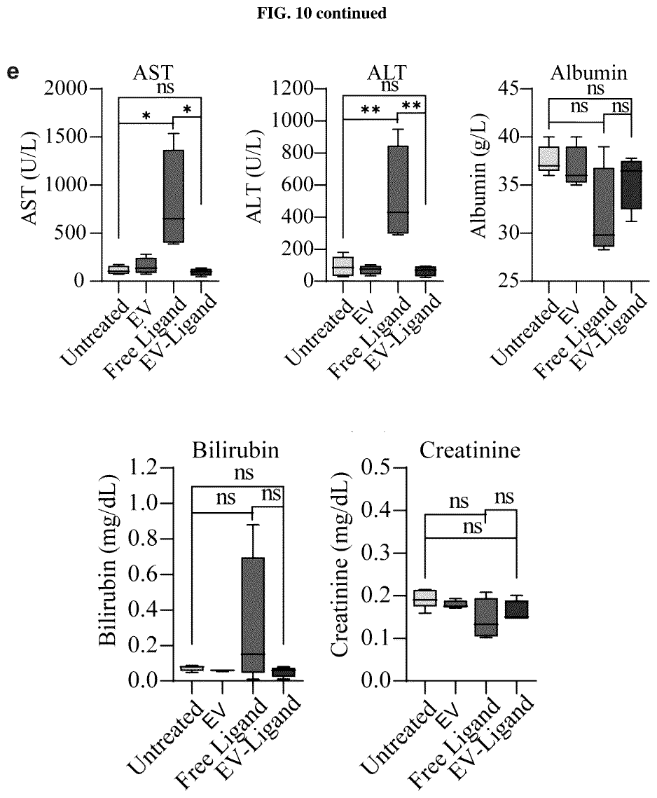

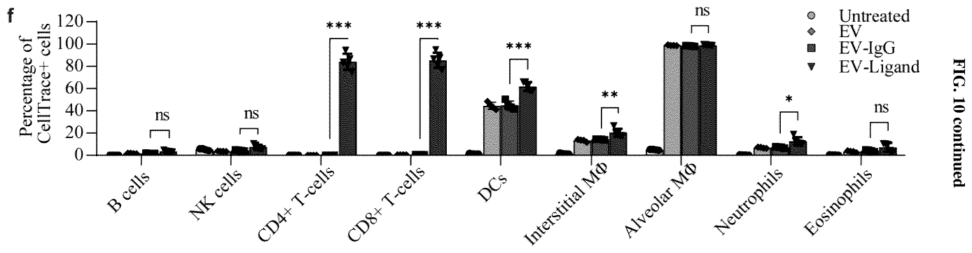

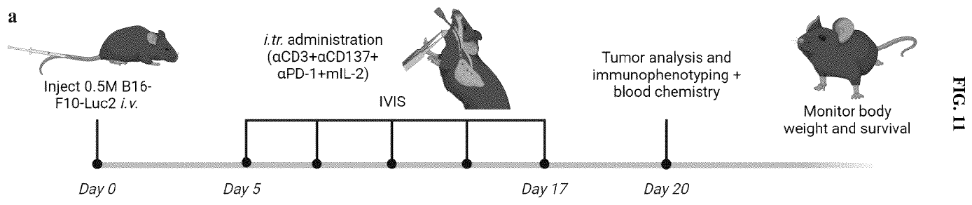

To assess the in vivo efficacy of the ligand functionalised EVs as disclosed herein, a lung metastatic model of melanoma that forms pulmonary metastases upon intravenous injection was used. This model mimics the metastasis of primary tumours into the lung, which are responsible for most fatalities in cancers such as melanoma. Treatments were administered directly into the lungs of mice via intratracheal administration (FIG. 10(a)). It was demonstrated that this method of administration can efficiently distribute EV suspensions throughout the lung at high coverage with a single dose. It also results in a high local concentration of the treatment in the immediate vicinity of the tumour metastases in the lung. Moreover, in the context of EV-based treatments, it has the advantage of overcoming clearance by the reticuloendothelial system, a phenomenon often observed with intravenous administration.

To assess the in vivo biodistribution of each treatment, free ligand and EV-Ligand treatments were labelled with a fixable near-infrared (IR) dye and a single dose was administered intratracheally. Four hours post-administration, the distribution of each treatment was assessed by tracking the near-IR signal using an in vivo imaging system (IVIS). The EV-Ligand treatment was shown to almost completely localise to the lungs, while the Free Ligand treatment accumulated prominently in the serum and to a lesser extent in the GI tract and liver (FIG. 10(b) to (c)). Without being bound by theory, it is thought that the larger size of the EVs slows down their permeation through tissues, thereby limiting their exposure to systemic circulation. Free ligands and antibodies are smaller and can diffuse through the lung tissue faster and enter the blood stream more readily. Further analysis revealed that the free ligand accumulated in the circulation of mice and remained at high levels for up to 42 hours, possibly owing to the Fc domains on the antibodies (FIG. 10(d)). The presence of EV-Ligand was not detected in the serum at any of the tested timepoints. This data indicates that ligands conjugated onto EVs are retained near the site of administration, reducing exposure to systemic circulation and other organs. Consequently, this decreases the likelihood of side effects by preventing ligands from interacting with tissues elsewhere in the body.

Given the broad biodistribution of the free ligands, a series of toxicity studies were performed to detect possible adverse effects induced by repeated doses of free ligand or EV-Ligand treatments. The free ligand treated mice showed significant elevations in serum transaminase levels including serum aspartate aminotransferase (AST) and alanine aminotransferase (ALT), indicative of liver toxicity. Additionally, non-significant perturbations in albumin, bilirubin and creatinine content in Free Ligand treated mice were observed (FIG. 10(e)). None of the EV-ligand treated mice showed any changes in these toxicity parameters from baseline levels, indicating that EV-associated ligands display an improved safety profile. This is in line with other EV studies that report improved toxicity profiles due to the altered biodistribution of ligands displayed on the EV surface. Further investigation into the precise cellular uptake of EV-Ligand treatments (conjugated with αCD3, αCD137, αPD-1 and mIL-2) via flow cytometric immunophenotyping of lung cells revealed high levels of EV-Ligand accumulation in CD4+ and CD8+ T-cells (FIG. 10(f)). In contrast, EVs conjugated with a control antibody (EV-IgG) showed little to no accumulation in T-cells. This data establishes that EV-Ligand treatments can specifically interact with target cells displaying cognate receptors. It was demonstrated herein that EVs were taken up predominantly by alveolar macrophages, interstitial macrophages and dendritic cells.

### EV-Bound Ligands Show Superior Immune Activation, Tumour Suppression and Safety Profiles Over Free Ligands

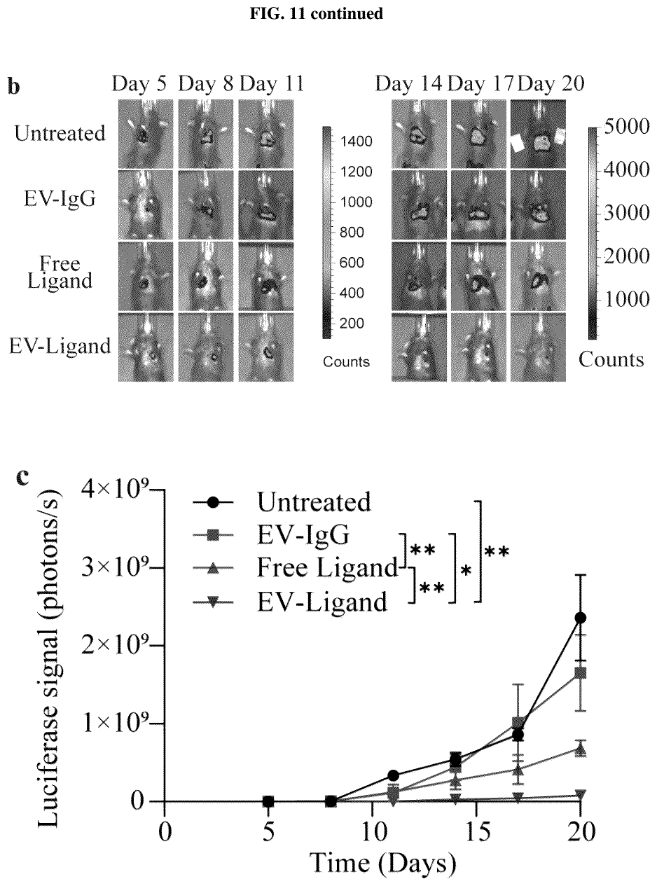

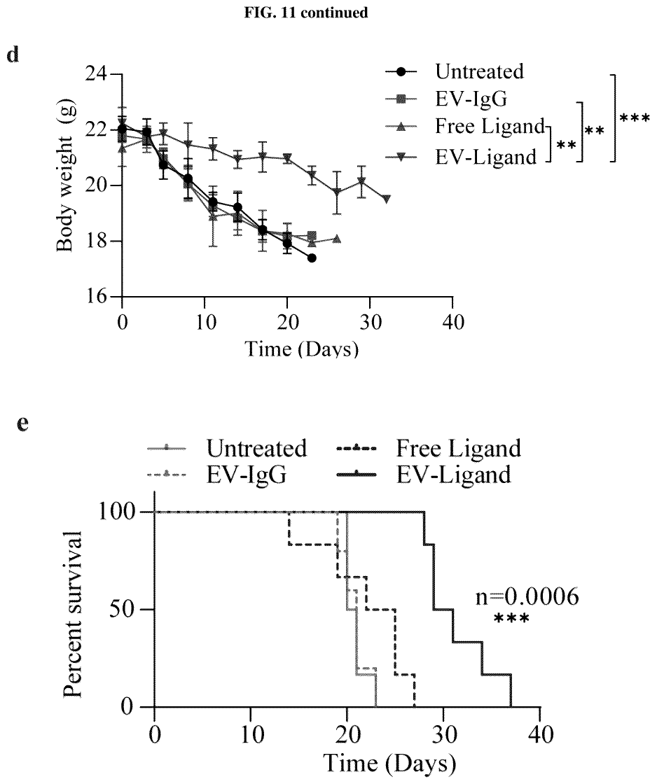

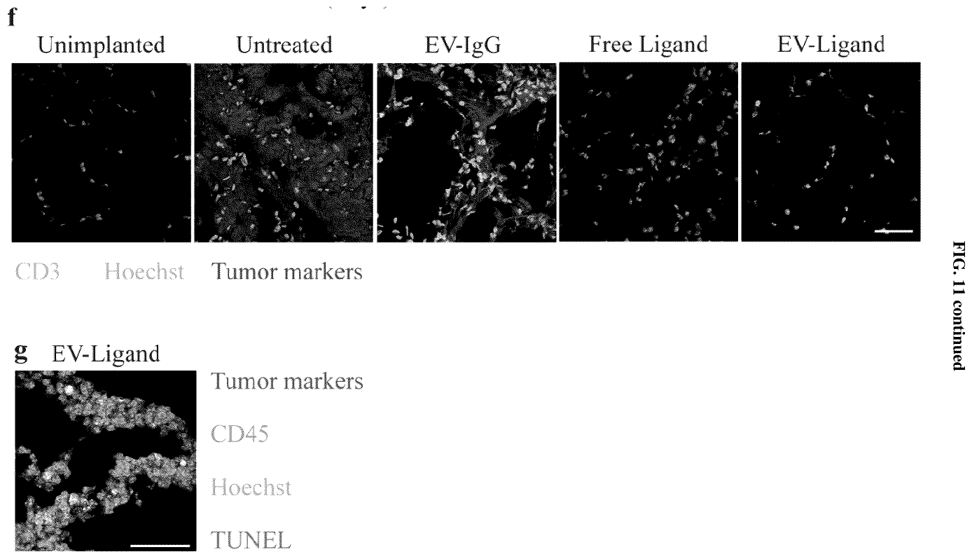

To assess the in vivo functionality of EV-Ligand treatments, a lung metastatic B16-F10 melanoma allograft expressing a Firefly luciferase reporter was developed by injecting 0.5 M B16-F10-Luc2 cells intravenously. The tumour cells were allowed to form micro metastases in the lungs of mice before treatments of EV-conjugated immunomodulatory ligands (EV-Ligand) or equivalent quantities of Free Ligand were administered intratracheally (FIG. 11(a)). Monitoring of tumour progression via IVIS revealed that the EV-Ligand and free ligand treated mice showed improved tumour suppression. However, the EV-Ligand showed significantly improved tumour suppression. Isotype control antibody conjugated EVs (EV-IgG) had no significant effect in suppressing tumour progression (FIG. 11(b) to (c)). It was also observed that mice treated with EV-Ligand showed significantly lower decreases in body weight for the duration of the study. Untreated, EV-IgG, and free ligand treated mice, by comparison, showed significant decreases in body weight (FIG. 11(d)). EV-Ligand treated mice also showed significantly improved survival, though all the mice did succumb to the tumour burden by day 33 post implantation. Free ligand treated mice also showed an improvement in overall survival, albeit less than that observed in the EV-Ligand treated mice (FIG. 11(e)). Immunofluorescence imaging of lung tumour sections from each treatment also revealed improved T-cell infiltration as evidenced by the increased CD3-positive cells in mice treated with EV-Ligand treatments. Free ligand treated mice showed slightly lower levels of T-cell infiltration. It was also observed a significant difference in tumour cell burden, with untreated mice showing higher levels of tumour burden as compared to both the Free Ligand and EV-Ligand treatments with the tumour burden in line with the IVIS data obtained previously (FIG. 11(f)). TUNEL staining of lung tumour sections from EV-Ligand treated mice was also performed, and a high level of TUNEL staining in tumour cells in EV-Ligand treated mice (FIG. 11(g)) was observed. Lastly, liver toxicity evaluation was performed by measuring the levels of ALT in the serum of mice following a full treatment regimen on Day 20 post implantation. Upregulation of ALT levels in the free ligand treatment was observed as compared to EV-Ligand or untreated mice, which is in line with toxicities for combination immunotherapeutics in their free form (FIG. 11(h)). These data together indicate that EV-Ligand treatments induce superior immune activation and tumour suppression while negating toxic off-target side effects observed with most immunotherapeutics.

In one example, the EVs disclosed herein can be modified to comprise up to 30 different ligands. In another example, the EVs disclosed herein can be modified to comprise between 10 to 25 different ligands. Experimental data for up to 5 molecules concurrently conjugated has been shown in FIG. 7(A). In this example, the combination of ligands used comprises agonistic CD137 antibody, agonistic CD3 antibody, recombinant mouse IL-2 and antagonistic PD-1 antibody and antagonistic CTLA-4 antibody, all of which were conjugated simultaneously on a single EV and demonstrated tumour cell killing activity both in vitro and in vivo (FIGS. 7 and 11).

Disclosed herein is a combination of five immunomodulatory ligands that were designed to address inhibitory factors and enhance T-cell-mediated anti-tumour responses. This combination included agonistic CD3 and CD137 antibodies, recombinant mouse IL-2 and antagonistic PD-1 and CTLA-4 antibodies (FIG. 7(a)). These EVs were termed ImmEVs or EV-Ligand. These EVs were shown to be able to induce improved immune activation and anti-tumour effects ex vivo (FIG. 7(c) to (f)). Moreover, treatment with ImmEVs resulted in improved tumour suppression in a mouse model of lung metastatic melanoma (FIG. 11).

Intraluminally Loaded Immune Agonists Benefit from EV-Mediated Delivery and Synergize with Surface Display of Immunomodulatory Ligands

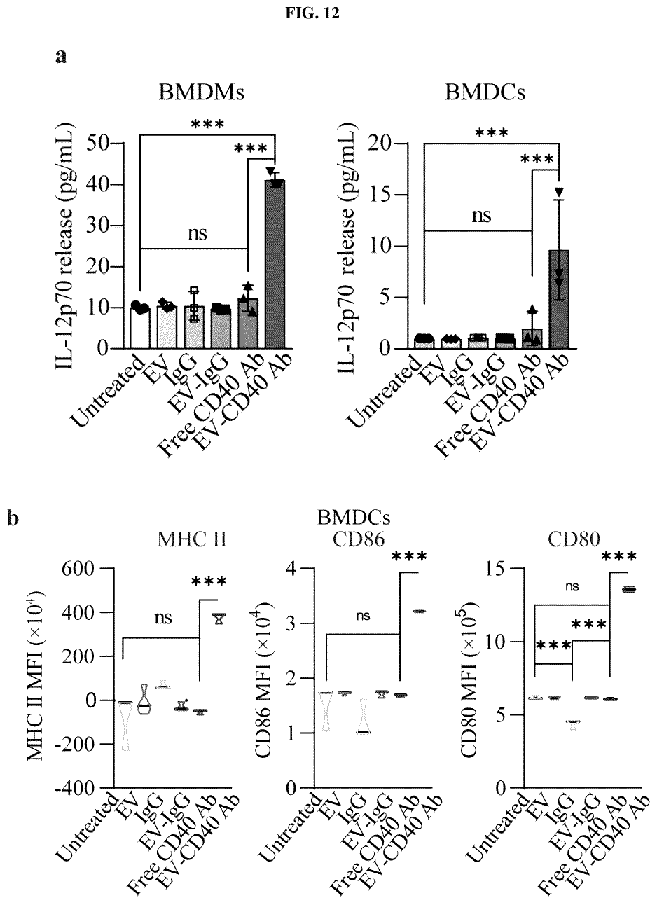

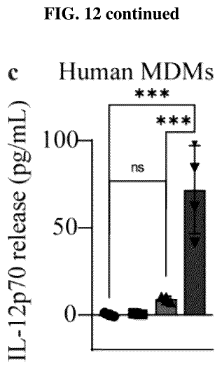

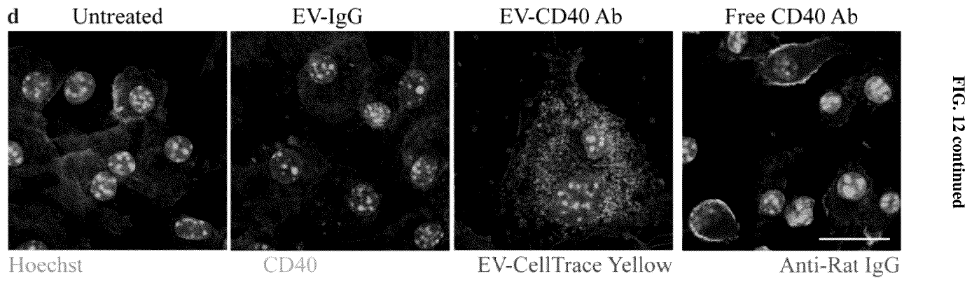

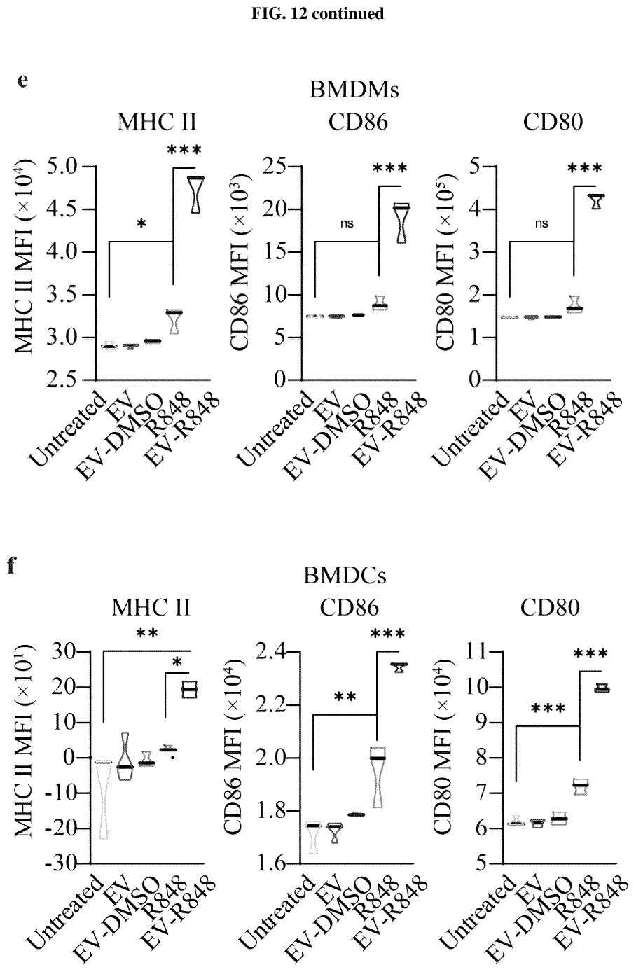

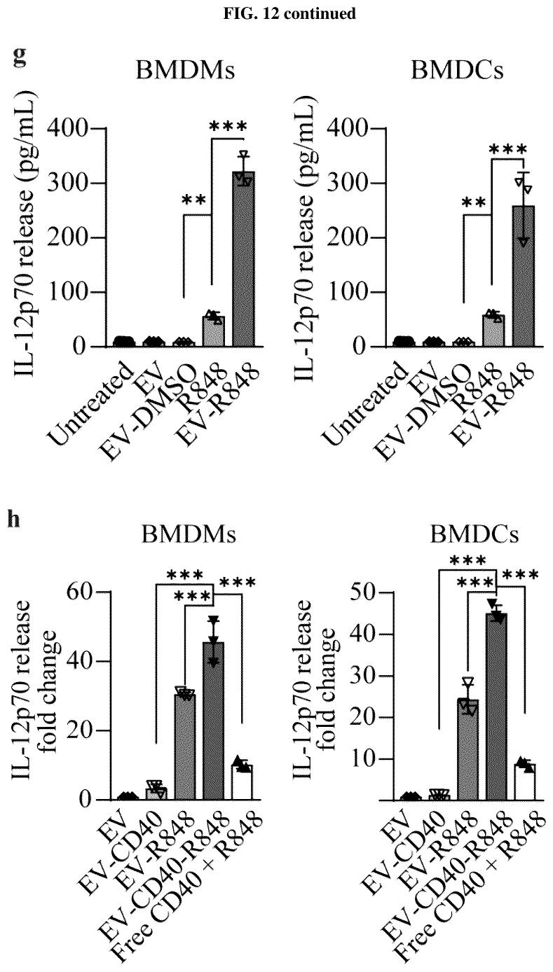

It was previously observed that EVs were readily taken up by phagocytic cells upon intratracheal administration, principally by macrophages and dendritic cells. These cells express CD40, a crosslinking-dependent member of the Tumour Necrosis Factor Receptor Superfamily (TNFRSF), which plays a role in modulating anti-tumour immune responses and is a target for immunotherapy. Thus, as disclosed herein are EVs conjugated with CD40 agonists that can be used to achieve effective CD40 signalling in these cells. The data demonstrates that conjugation of an agonistic CD40 antibody on the surface of EVs significantly enhanced its activity compared to equivalent doses of the free antibody. This is evidenced by the significant increases in IL-12 release and upregulation of MHC II and co-stimulatory molecules on primary murine bone marrow-derived macrophages (BMDMs) and bone marrow-derived dendritic cells (BMDCs) respectively (FIG. 12(a) to (b)).

To confirm that EV-mediated enhancements were translatable to human models, CD40 stimulation was repeated in human monocyte-derived macrophages (MDMs) using a human-specific CD40 agonistic antibody. The data revealed similar enhancements in IL-12 release by human MDMs upon EV-CD40 Ab treatment (FIG. 12(c)). The effects of CD40 agonist-conjugated EVs on CD40 clustering were further investigated using confocal imaging. Using a polyclonal CD40 antibody raised against the cytoplasmic domain of CD40, it was verified that CD40-conjugated EVs effectively engaged CD40 receptors and induced clustering and internalization (FIG. 12(d)). In contrast, free CD40 antibody showed minimal clustering of CD40 receptors. This suggests that ligand-conjugated EVs can effectively engage cognate receptors and induce clustering and subsequent crosslinking to achieve more efficient signalling.

The immunofluorescent data had shown that upon receptor engagement, EVs were eventually endocytosed by the target cells (FIG. 12(d)). Thus, innate immune agonists were loaded into the lumen of EVs to activate innate immune pathways in recipient cells upon endocytosis. It has been shown CD40 signalling synergizes well with Toll-like receptor (TLR) agonists. Thus, disclosed herein is the utility of R848 (Resiquimod), an innate immune agonist of the TLR7/8 pathway that is commonly used in cancer immunotherapy, in enhancing the immune stimulatory effects of CD40 agonism. Of note, TLR7/8 receptors are present in the endosomal membrane compartments, making EVs ideal vectors to deliver R848. R848 was loaded into EVs via the use of DMSO-mediated permeabilisation of the EV membrane, resulting in approximately 10 ng of R848 encapsulated per μg of EV. Subsequently, the efficacy of RBCEV loaded R848 to an equivalent dose of free R848 was compared by quantifying the increase in expression of CD80, a co-stimulatory molecule expressed on RAW264.7 cells. The data revealed that the EV-loaded R848 outperformed the free R848 across all tested doses. More detailed investigation in primary BMDMs and BMDCs revealed that EV-R848 resulted in increases in the expression of MHC II, CD86, CD80 and the release of IL-12 as compared to equivalent doses of free R848 (FIG. 12(e) to (g)). This is attributed to the phagocytic capacity of macrophages and dendritic cells for EVs, particularly those containing antibodies targeting cellular receptors, which results in efficient accumulation of loaded R848 in the endosomal compartments of these cells.

The effect of co-treating cells with both R848 and CD40 was examined. CD40-conjugated EVs loaded with R848 (EV-CD40-R848) elicited significantly higher IL-12 release from both BMDMs and BMDCs compared to the combined IL-12 release from cells treated with EVs loaded with R848 alone or EVs conjugated with CD40 antibody alone, indicating the presence of a synergistic relationship between R848 and CD40 agonism. It was further shown that treatment with EV-CD40-R848 was more effective than co-treatment with equivalent doses of free CD40 and R848 (FIG. 12(h)).

In one example, the nanoscale extracellular vesicle is as disclosed herein, wherein the agonistic antibody is anchored to the nanoscale extracellular vesicle, and wherein the nanoscale extracellular vesicle comprises a payload. In another example, the agonistic antibody is anti-CD40 antibody, and the payload is R848 (Resiquimod).

In one example, described herein is a method of treating or preventing a disease, the method comprising administering a composition comprising one or more nanoscale extracellular vesicles as disclosed herein to a subject. In one example, the nanoscale extracellular vesicle is conjugated with an agonistic antibody, anti-CD137 antibody, and an antagonistic antibody, anti-PD-L1 antibody. In another example, the nanoscale extracellular vesicle is conjugated with an agonistic antibody, anti-CD40 antibody, and comprises a payload, R848.

### EV-Mediated Delivery of Immunomodulatory Ligands Results in Improved Anti-Tumour Immune Responses and Improved Overall Survival in an Autochthonous Cell-Derived Pancreatic Ductal Adenocarcinoma (PDAC) Model

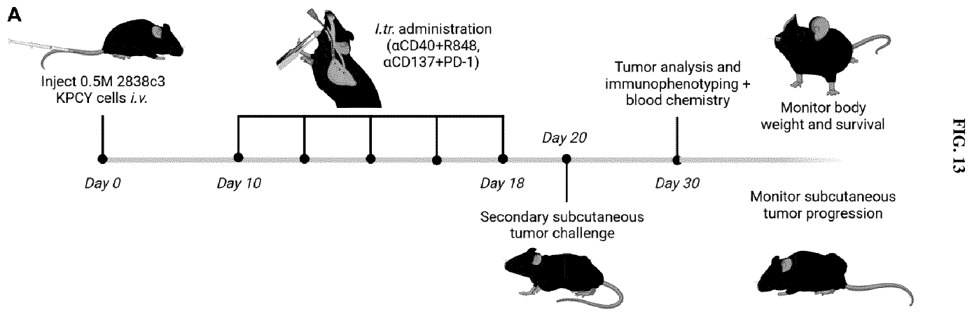

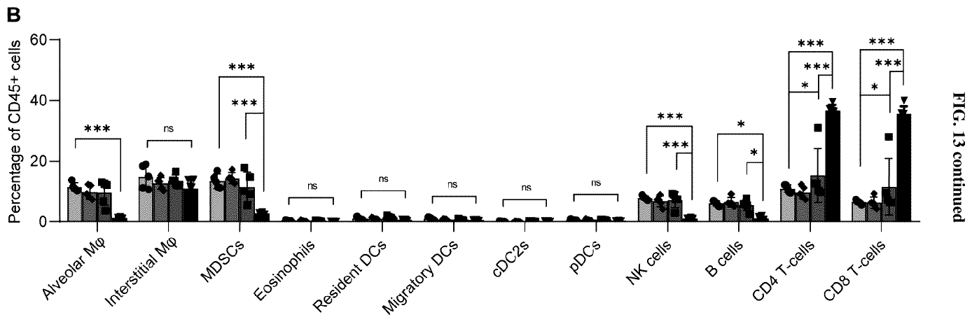

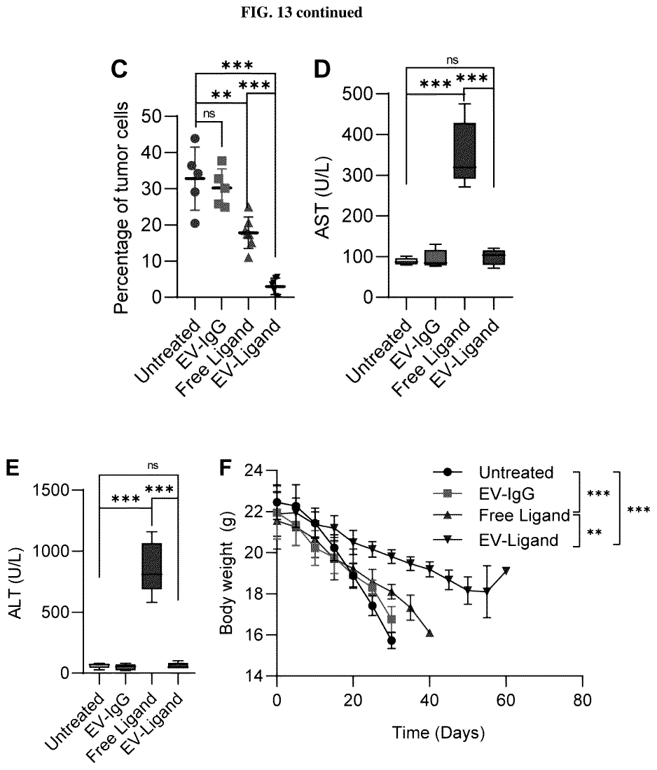

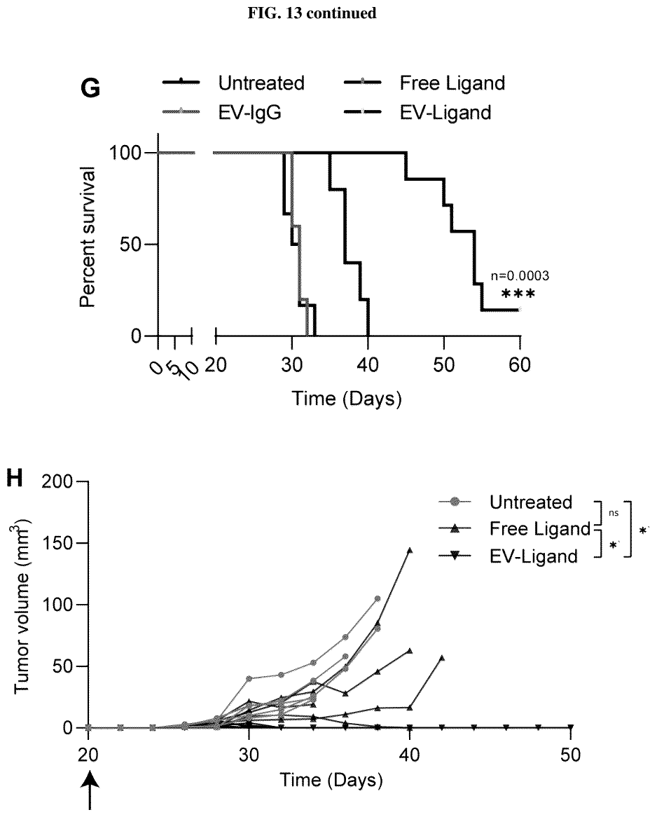

To further validate the efficacy of the EV-based strategy as disclosed herein for the delivery of immunomodulatory ligands, an in vivo experiment was conducted using a lung metastatic autochthonous cell-derived PDAC model. KPCY 2838c3 tumour cells were found to express PD-L1 and high levels of a YFP reporter. Previous studies using similar models of PDAC have demonstrated effective tumour control via the use of a combination of agonistic CD137 antibodies, PD-1 blockade, agonistic CD40 antibodies and TLR stimulation. Thus, disclosed herein are the two distinct EV treatments, namely, 1) EVs displaying αCD137 and αPD-1 antibodies targeted to T-cells; and 2) EVs displaying αCD40 antibodies and loaded with R848 targeted to dendritic cells and macrophages. Mice were treated every other day with five doses of EV-based or free ligand formulations 10 days post-implantation. Immune cell composition, tumour burden, and toxicity were assessed on day 30 (FIG. 13(a)).

Immunophenotyping via multicolour flow cytometry revealed changes in immune cell composition following EV-Ligand treatments, as illustrated in the t-distributed stochastic neighbor embedding (tSNE) plots in FIG. 6(b). Mice treated with the EV-Ligand treatment displayed significant increases in both CD4 and CD8 T-cells (FIG. 13(b)). However, no changes were observed in the percentage of DCs or interstitial macrophages upon treatment. The increase in T-cells was accompanied by decreases in immune suppressive immune cell subsets, namely, MDSCs and Tregs. A decrease in the relative percentage of B cells, NK cells and alveolar macrophages following EV-Ligand treatment was also observed. Without being bound by theorz, it is thought that this was attributed to an increase in the proportion of T-cells rather than an actual reduction in these immune cell types. Other changes to the tumour immune milieu upon treatment with EV-Ligand included increased expression of Granzyme in CD8+ T-cells, upregulation of MHC II in DC subsets, increased M1 polarization of interstitial macrophages and enhanced expression of IFN-γ mRNA, all of which were elevated compared to treatment with equivalent doses of free ligand treatments. Increases in IFN-α, IFN-β, or IL-12 were not detected, although an increase in IL-12 mRNA levels following EV-Ligand treatment was observed.

Taking into account the alterations to the percentage of T-cells in the tumour between different treatment groups, further analysis of T-cell markers involved in the generation of anti-cancer immune responses was performed. Detailed analysis of CD69, CD25, PD-1, FoxP3 and Granzyme B expression in T-cells revealed that EV-Ligand treated mice displayed enhanced T-cell activation, higher T-cell cytotoxicity, and depleted regulatory T-cells. Taken together, the data disclosed herein shows that EV-mediated delivery of immunomodulatory ligands can effectively remodel the tumour immune environment.

The changes in immune cell composition observed in the EV-Ligand treatment group were accompanied by a significantly lower tumour burden compared to mice treated with an equivalent dose of the Free Ligand or control EVs (FIG. 13(c)). While free ligand treated mice displayed suppression of tumour growth compared to control mice, this was accompanied by an increase in serum AST and ALT levels, indicating significant levels of liver toxicity (FIG. 13(d) to (e)). Further experiments also revealed that EV-Ligand treated mice consistently maintained a higher body weight throughout the treatment duration compared to all other treatment groups (FIG. 13(f)). This was also shown in the survival study, where EV-Ligand treated mice showed a pronounced improvement in overall survival, with one mouse showing complete tumour remission by day 60 (FIG. 13(g)). Meanwhile, free ligand treatments only conferred moderate improvements in survival over control mice. In a separate experiment, the development of tumour-specific immune memory was assessed by injecting KPCY 2838c3 cells subcutaneously on day 15 and monitoring tumour volume over the next 20 days. None of the EV-Ligand treated mice developed flank tumours, indicative of the generation of antigen-specific immune activation induced by EV-Ligand treatments (FIG. 13(h)). In contrast, only one of the five free ligand treated mice showed complete remission upon secondary tumour challenge, while the remaining mice displayed tumours that were smaller (on average) than the control mice. This data demonstrates that EV-based delivery enhances the efficacy of immunomodulatory ligands. This, in turn, results in an increase in tumour-specific immune responses, while concurrently abrogating the onset of systemic toxicity, conferring significant improvements in overall survival.

In one example, hundreds of copies of each ligand can be conjugated on a single EV. Another limiting factor to the number of ligands that can be anchored onto a single EV would be the physical constraint represented by the finite surface area provided by an EV of any specific size. Thus, in one example, the EVs disclosed herein comprise between 1 to 30 ligands or 10-25 ligands, or 2, 3, 4, 5, 6, 7, 8, 9, 10, 11, 12, 13, 14, 15, 16, 17, 18, 19, 20, 21, 22, 23, 24, 25, 26, 27, 28, 29, or 30 ligands. The number of ligands that can be conjugated into the EV surface depends on the size of the ligand. For example, antibodies occupy more space on a surface, and would therefore limit the maximum copy number of ligands that can be conjugated onto the surface of the EV. On the other hand, small cytokines/ligands are much smaller, allowing many more to be conjugated onto the surface of an EV.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Material and Methods

### Red Blood Cell-Derived Extracellular Vesicle (RBCEV) Purification and Characterization

Red blood cell-derived extracellular vesicles (RBCEVs) were purified and isolated from human red blood cells (RBCs) as previously described in Usman, W. M. et al. (Efficient RNA drug delivery using red blood cell extracellular vesicles. Nat Commun 9, 2359 (2018)). The present disclosure utilises extracellular vesicles (EVs) purified from human RBCs, also referred to herein as EVs. However, this should not be considered limiting, as EVs obtained from other sources can also be used. Purified EVs were characterized using western blot, nanoparticle analysis and single EV flow cytometry.

For western blot analysis, equal quantities of total protein from RBCEV lysates and RBC lysates were electrophoresed in 15-20% polyacrylamide gels and probed with relevant antibodies. Nanoparticle tracking analysis was performed using a NanoSight NS3000 system (Malvern) or a Zetaview Particle analyser (Particle Metrix). In brief, EV suspensions were diluted 1000-fold and analysed using the machine. Three independent repeats using EVs from 3 independent donors was used for each reading. Single EV flow cytometric analysis was performed using a NanoFCM system (NanoFCM). EVs were stained for 30 minutes with antibodies/fluorescent probes before being diluted 10-fold and spun down to remove excess antibody. The resulting EVs were resuspended, diluted 50-fold, and analysed using the NanoFCM system, recording all events for a total duration of 1 minute.

### EV Conjugation and Functionalisation

Purified RBCEVs were functionalised with either STP/NHS/TFP amine reactive esters or Maleimide crosslinkers with the aim of introducing relevant click handles onto the EV surface. Similarly, target proteins that were to be conjugated on to the EV surface were similarly labelled with a complementary click handle. Alternatively, biotin-STP/streptavidin was used to biotinylate and crosslink EVs to target proteins. Following installation of click handles labelled EVs and proteins were repurified to remove unreacted esters and click handles. EVs were washed by centrifugation for a total of 4 times. Proteins were subjected to buffer exchange via centrifugal filter, buffer exchange columns or dialysis. Click handles installed included the SPAAC pair (Azide/DBCO) and the iEDDA pair (Methyltetrazine/Transcyclooctene).

Purified RBCEVs labelled with click handles were incubated with proteins labelled with a corresponding click handle to facilitate the click reaction to form EVs functionalised with the desired protein. For the conjugation of multiple ligands on the EV surface, proteins were incubated at the concentration ratio desired for conjugation.

### Determination of Protein Conjugation Efficiency and Copy Number Per Extracellular Vesicle (EV)

The copy number per EV was determined through the following steps: First, ELISA was used to quantify the copy number of conjugated proteins in a given volume of EVs. For example, about 1 μL of diluted EVs was lysed, and the total quantity of conjugated protein molecules within these EVs was measured using ELISA. Next, the same diluted EV suspension was analysed using a nanoparticle tracking analysis instrument to determine the EV concentration, which allowed us to calculate the number of EVs in the 1 μL suspension. Finally, an estimate of the average copy number of conjugated molecules per single EV can be obtained by dividing the total number of conjugated proteins by the number of EVs present in the 1 μL suspension.

### Ex Vivo Functional Assays

Splenocytes for ex vivo experimentation were obtained from the spleens of healthy mice. In brief, spleens were excised from mice immediately after sacrificing and subjected to enzymatic digestion using a Miltenyi dissociator and Collagenase IV. The resulting cell suspension was centrifuged to remove debris and undigested tissue. RBCs were lysed using ACK lysis buffer. The resulting cell pellet was washed and cultured in Roswell Park Memorial Institute (RPMI) 1640 supplemented with 10% heat inactivated fetal bovine serum (FBS), 100 U/mL IL-2 and 0.05 mM 2-mercaptoethanol. T-cells were sorted from the splenocytes using an untouched T-cell isolation kit (Thermo Fisher Scientific) following the manufacturer's instructions.

Human peripheral blood mononuclear cells (PBMCs) for ex vivo experimentation were obtained via isolation from apheresis samples obtained from the Health Sciences Authority of Singapore from individuals with informed consent. Isolation of PBMCs was performed by gradient density separation using Ficoll-Paque Plus (Cytiva, USA) and low-speed centrifugation. Cells were washed with RPMI 1640 supplemented with 10% heat-inactivated FBS and 1% penicillin/streptomycin (Thermo Fisher Scientific, USA).

To assess cytokine release by T-cells following CD3 or CD137 stimulation. T-cells were activated using αCD3-αCD28 beads prior to being incubated with free soluble αCD3 or αCD137 agonistic antibody; or EV-conjugated αCD3 or αCD137 antibodies for 24-48 hours. The resulting cell suspension was spun down to remove cells and the supernatant assayed for interferon gamma and IL-2 using human and mouse interferon gamma and IL-2 ELISA kit (BioLegend).

To assess T-cell activation potential of different treatments, non-activated and/or activated T-cells were treated for a total of 6 days. The presence of activation markers (CD69, CD25) were assessed on day 1, 3, and 5) using flow cytometric analysis. At the endpoint (Day 5), the expression of other relevant markers such as Granzyme B were assessed using intracellular flow cytometry. Proliferation of T-cells during the experiment was tracked using alamarBlue assay and CellTrace dilution assay.

For the ex vivo tumour cell killing assay, tumour reactive splenocytes were isolated from B16-F10-Luc2 tumour bearing mice that had been implanted with a 0.5 million tumour cells intravenously 20 days prior. Splenocytes were obtained as described before while CD8⁺ cytotoxic T-cells were isolated from lung tumour homogenates using CD8 (TIL) MicroBeads, mouse (Miltenyi). Immune cells were subsequently co-cultured with B16-F10-Luc2 cells expressing a firefly luciferase reporter and incubated with desired treatments. Tumour cell death was assayed at 3 days and 5 days using luciferase assay (Promega) following the manufacturer's instructions. At the endpoint of the experiment, the expression of T-cell markers such as Ki-67 and Granzyme B were determined using intracellular flow cytometry. For ex vivo tumour cell killing assay with bispecific EVs, immune cells were co-cultured with H1975 cells expressing a firefly luciferase reporter and incubated with desired treatments. Tumour cell death was assayed after 48 hours using luciferase assay using Tecan Spark 10 M microplate reader (Tecan, Switzerland).

### In Vivo Experiments

All in vivo experiments were conducted according to protocols approved by the Institutional Animal Care and Use Committee under the National University of Singapore. Mice were injected intravenously with 0.5 million B16-F10-Luc2 cells. The tumours were allowed to grow for 5 days before being measured using IVIS to confirm tumour implantation in the lung. Mice with similar tumour burden were used for subsequent in vivo treatment experiments. Mice were grouped randomly into each treatment group and administered with the treatment every 3 days (days 5, 8, 11, 14, 17). The treatment for each mouse was made up to 60 μL and administered intratracheally. Tumour burden (using IVIS) and bodyweight were also monitored at 3-day intervals. For the monitoring of tumour burden the study was ended on day 20 and the lungs of mice were excised for immunofluorescent imaging of immune activation and tumour burden. For the survival study, mice were monitored following conclusion of the treatment until they reached the criteria set out for symptom free survival (a 20% drop in body weight or severe loss of mobility or cachexia).

For in vivo characterisation of bispecific EVs, the NOD-scid IL2Rγnull (NSG-SGM3) mice were injected intravenously with 1 million H1975-mCherry-luciferase cells. Tumour development was monitored using IVIS to confirm tumour inoculation in the lung. After 3 weeks, mice with similar tumour burden were intravenously injected with 10 million human PBMCs, followed by a single dose of unmodified EVs, IgG-conjugated EVs or BEVs at 5 mg/kg. Mice were sacrificed after 3 hours and organs were harvested for ex vivo imaging, immunofluorescent imaging of T cell infiltration and flow cytometric analysis of T cell activation.

### Statistical Analysis

GraphPad Prism 8 was used to conduct all statistical analyses. One-tailed Student's t-test was used to assess significance levels between controls and experimental samples. For analysing the difference among multiple treatment groups, two-way ANOVA was utilized. Throughout this study, a p-value<0.05 was considered to be significant. Data in the graphs are represented as the mean, with error bars indicating the standard deviation. Each experiment was repeated at least three times using RBCEVs from independent donors and/or cells from different passages. All data pertaining to animal work are presented as the mean from a minimum of 5 individual mice.

1. A nanoscale extracellular vesicle for immunotherapy, wherein one or more types of immunomodulatory molecules is/are anchored to the nanoscale extracellular vesicle, wherein the types of immunomodulatory molecules are selected from the group consisting of agonistic antibodies, antagonistic antibodies, cytokines, chemokines, small molecules, agonistic ligands, and combinations thereof; and/or
wherein the cytokines are selected from the group consisting of IL-1, IL-2, IL-3, IL-4, IL-5, IL-6, IL-8, IL-9, IL-10, IL-11, IL-12, IL-13, IL-16, IL-17, IL-18, IL-23, IL-33, IL-1a, IL-13, TNF-α, TNF-β, and combinations thereof; and/or wherein the cytokine is IL-2; and/or
wherein the chemokines are selected from the group consisting of CCL2, CCL3, CCL4, CCL5, CCL7, CCL8, CCL11, CCL13, CCL20, CCL22, CCL24, CCL26, CCL28, CXCL1, CXCL8, CXCL12, CXCL13, XCL1, CX3CL1, and combinations thereof; and/or
wherein the agonistic antibodies are selected from a group consisting of anti-CD3, anti-CD137, anti-CD28, anti-CD27, anti-OX-40, anti-CD357, anti-CD40, anti-CD278 antibodies, and combinations thereof; and/or wherein agonistic antibodies are selected from a group consisting of anti-CD3, anti-CD137, and anti-CD28 antibodies; and/or
wherein the antagonistic antibodies are selected from a group consisting of anti-PD-1, anti-PD-L1, anti-CTLA-4, anti-CD96, anti-TIM-3, anti-LAG-3 antibodies, and combinations thereof; and/or wherein the antagonistic antibodies are selected from a group consisting of anti-PD-1 and anti-CTLA-4; and/or
wherein the small molecules are selected from a group consisting of STING agonists, TLR7 agonists, and combinations thereof; and/or wherein the STING agonists are selected from a group consisting of STG-982, STG-968, and combinations thereof; and/or wherein the TLR7 agonists are selected from a group consisting of TL7-887. TL7-975, and combinations thereof; and/or
wherein the agonistic ligands are selected from a group consisting of TNF, LT, CD27L CD137L, GITRL, OX40L, CD40L, CD30L, herpes simplex virus glycoprotein D, TNFSF14, LTα, BAFF, APRIL, GITRL, TL1A, and combinations thereof; and/or wherein the agonistic ligand is CD137L; and/or
wherein the agonistic ligands are in soluble form; and/or wherein the extracellular vesicle comprises click chemistry handles; and/or, wherein the click chemistry handle is selected from the group consisting of dibenzocyclooctynes (DBCO), transcyclooctene (TCO), methyltetrazine (MTet), tetrazines, azide, alkynes, 9,10-phenanthrenequinones (PQs), electron-rich vinyl ethers (VEs), thiols, and maleimides; and/or, wherein the click chemistry handle is methyltetrazine (MTet), transcyclooctene (TCO), or a variant thereof.

2.-16. (canceled)

17. The nanoscale extracellular vesicle of claim 1, wherein 2, 3, 4, 5, 6, 7, 8, 9 10, 11, 12, 13, 14, 15, 16, 17, 18, 19, 20, 21, 22, 23, 24, 25, 26, 27, 28 or more types of immunomodulatory molecules are anchored to the nanoscale extracellular vesicle.

18. The nanoscale extracellular vesicle of claim 1, wherein the one or more immunomodulatory molecules are anchored to the nanoscale extracellular vesicle using a corresponding click chemistry handle selected from the group consisting of dibenzocyclooctynes (DBCO), transcyclooctene (TCO), methyltetrazine (MTet), tetrazines, azide, alkynes, 9,10-phenanthrenequinones (PQs), electron-rich vinyl ethers (VEs), thiols, and maleimides, wherein the click chemistry handle and the corresponding click chemistry handle are different from each other; and/or wherein the corresponding click handle is a transcyclooctene (TCO) click chemistry handle; and/or wherein the immunomodulatory molecules are bound to the nanoscale extracellular vesicle surface in cis-conformation.

19. (canceled)

20. The nanoscale extracellular vesicle of claim 1, wherein two types of immunomodulatory molecules are anchored to the nanoscale extracellular vesicle; and wherein the first type of immunomodulatory molecule is an agonistic antibody, and the second type of immunomodulatory molecule is an antagonistic antibody.

21. The nanoscale extracellular vesicle of claim 20, wherein the agonistic antibody is anti-CD3 antibody, and the antagonistic antibody is anti-PD-L1 antibody; and/or wherein both the anti-CD3 antibody and the anti-PD-L1 antibody comprise a transcyclooctene (TCO) click handle.

22. The nanoscale extracellular vesicle of claim 20, wherein the agonistic antibody is anti-CD137 antibody, and the antagonistic antibody is anti-PD-L1 antibody.

23. (canceled)

24. The nanoscale extracellular vesicle of claim 1, wherein three types of immunomodulatory molecules are anchored to the nanoscale extracellular vesicle; wherein the first type of immunomodulatory molecule is an agonistic antibody, the second type of immunomodulatory molecule is an antagonistic antibody, and the third type of immunomodulatory molecule is a cytokine; and/or wherein the agonistic antibody is CD3 and CD137 antibodies; wherein the antagonistic antibody is PD1 and CTLA-4 antibodies; and wherein the cytokine is IL-2.

25.-26. (canceled)

27. The nanoscale extracellular vesicle of claim 1, wherein the size of the nanoscale extracellular vesicle, excluding the click chemistry handles which are anchored to the nanoscale extracellular vesicle, ranges between 10 to 1000 nm; and/or wherein the size of the nanoscale extracellular vesicle, excluding the click chemistry handles which are anchored to the nanoscale extracellular vesicle, ranges between 50 to 400 nm; and/or wherein the size of the nanoscale extracellular vesicle, excluding the click chemistry handles which are anchored to the nanoscale extracellular vesicle, is about 160 nm.

28.-29. (canceled)

30. The nanoscale extracellular vesicle of claim 1, wherein the nanoscale extracellular vesicle originates from a cell or a compound capable of forming extracellular vesicles; and/or wherein the cell is selected from the group consisting of a red blood cell and a cancer cell; and/or wherein the compound is selected from the group consisting of milk and a plasmid; and/or wherein the cell is a red blood cell.

31.-32. (canceled)

33. The nanoscale extracellular vesicle of claim 1, wherein the nanoscale extracellular vesicle further comprises a payload; and/or wherein the payload is selected from a group consisting of small molecules and synthetic DNA molecules; and/or wherein the small molecules are selected from a group consisting of R848, imiquimod, and Muramyl dipeptide; and/or wherein the small molecule is R848; and/or wherein the synthetic DNA molecule is CpG ODNs.

34.-37. (canceled)

38. The nanoscale extracellular vesicle of claim 33, wherein the agonistic antibody is anchored to the nanoscale extracellular vesicle, and wherein the nanoscale extracellular vesicle comprises a payload; and/or wherein the agonistic antibody is anti-CD40 antibody and the payload is R848.

39. (canceled)

40. A method of obtaining a nanoscale extracellular vesicle for immunotherapy according to claim 1, the method comprising:
a. isolating nanoscale extracellular vesicles from a cell or a compound capable of forming extracellular vesicles;
b. attaching a click chemistry handle to the nanoscale extracellular vesicle surface;
C. attaching a corresponding click chemistry handle to the immunomodulatory molecule; and
d. conjugating the click chemistry handle of step b to the corresponding click chemistry handle of step c; thereby anchoring the immunomodulatory molecules to the surface of the nanoscale extracellular vesicle.

41. The method of claim 40, wherein the method further comprises:
i. permeabilising the nanoscale extracellular vesicle of step a. or step d. with dimethyl sulfoxide (DMSO); and
ii. incubating the nanoscale extracellular vesicle obtained from step i. with a payload.

42. The method of claim 40, wherein the method comprises two or more types of immunomodulatory molecules.

43. The method of claim 40, wherein the click chemistry handle and the corresponding click chemistry handle are selected from the group consisting of dibenzocyclooctynes (DBCO), transcyclooctene (TCO), methyltetrazine (MTet), tetrazines, azide, alkynes, 9,10-phenanthrenequinones (PQs), electron-rich vinyl ethers (VEs), thiols, and maleimides; and/or wherein the click chemistry handle and the corresponding click chemistry handle are conjugated via inverse electron-demand Diels-Alder (iEDDA), via strain-promoted alkyne-azide cycloadditions (SPAAC), via photoclick reactions (PQ-VE reactions), or via Maleimide crosslinking; and/or wherein the click chemistry handle and the corresponding click chemistry handle for inverse electron-demand Diels-Alder (iEDDA) are methyltetrazine and transcylcooctene, respectively; and/or wherein the click chemistry handle and the corresponding click chemistry handle for strain-promoted alkyne-azide cycloadditions (SPAAC) are azide and DBCO, respectively; and/or wherein the click chemistry handle and the corresponding click chemistry handle for photoclick reactions are 9,10-phenanthrenequinones (PQs) and electron-rich vinyl ethers (VEs), respectively; and/or wherein the click chemistry handle and the corresponding click chemistry handle for Maleimide crosslinking are thiols and maleimides, respectively.

44.-48. (canceled)

49. The method of claim 40, where the cell selected from the group consisting of a red blood cell and a cancer cell; and/or wherein the compound is selected from the group consisting of milk and a plasmid; and/or wherein the cell is a red blood cell.

50.-51. (canceled)

52. A method of treating or preventing a disease, the method comprising administering a composition comprising one or more nanoscale extracellular vesicles according to claim 1 to a subject.

53. The method of claim 52, wherein the agonistic antibody is anti-CD137 antibody, and the antagonistic antibody is anti-PD-L1 antibody.

54. The method of claim 52, wherein the nanoscale extracellular vesicle further comprises a payload; and/or wherein the payload is selected from a group consisting of small molecules and synthetic DNA molecules; and/or wherein the small molecules are selected from a group consisting of R848, imiquimod, and Muramyl dipeptide; and/or wherein the small molecule is R848; and/or wherein the synthetic DNA molecule is CpG ODNs.

55. The method of claim 52, wherein the disease to be treated or prevented is cancer and wherein the immunomodulatory molecules are suitable to treat or prevent cancer; and/or wherein the cancer is selected from the group consisting of lung cancers, breast cancers, colorectal cancers, prostate cancers, melanoma skin cancers, non-melanoma skin cancers, leukaemia, lymphomas, pancreatic cancers, ovarian cancers, cervical cancers, brain cancers, liver cancers, stomach cancers, and oesophageal cancers; and/or wherein the melanoma skin cancer is lung metastatic melanoma; and wherein the pancreatic cancer is pancreatic ductal adenocarcinoma.

56.-57. (canceled)
